(19) United States
(12) Patent Application Publication
(10) Pub. No.: US 2025/0179051 A1
(43) Pub. Date: Jun. 5, 2025

(54) PYRIMIDINLY-OXY-QUINOLINE BASED HERBICIDAL COMPOUNDS

(71) Applicant: SYNGENTA CROP PROTECTION AG, Basel (CH)

(72) Inventors: Vanitha ACHARYA, Corlim, Ilhas (IN); Sunil CHAKAVE, Corlim, Ilhas (IN); Mangala PHADTE, Corlim, Ilhas (IN)

(73) Assignee: SYNGENTA CROP PROTECTION AG, Basel (CH)

(21) Appl. No.: 18/842,578

(22) PCT Filed: Feb. 22, 2023
(86) PCT No.: PCT/EP2023/054446

§ 371 Date: Aug. 29, 2024

(30) Foreign Application Priority Data
Mar. 1, 2022 (IN) 202211010934

(51) Int. Cl. C07D 403/12; A01N 43/54; A01N 43/56; A01N 43/80; A01P 13/00; C07D 401/14; C07D 403/14; C07D 413/14

(52) U.S. Cl. CPC C07D 403/12; A01N 43/54; A01N 43/56; A01N 43/80; A01P 13/00; C07D 401/14; C07D 403/14; C07D 413/14

(57) ABSTRACT

The present invention relates to compounds of Formula (I), or an agronomically acceptable salt of said compounds wherein X¹, Y¹, Y², Z¹, Z², R¹, R², R⁵, R⁹ and n are as defined herein. The invention further relates to herbicidal compositions which comprise a compound of Formula (I) and to the use of compounds of Formula (I) for controlling weeds, in particular in crops of useful plants.

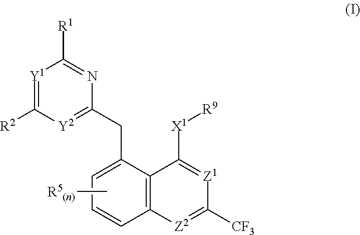

The present invention relates to novel herbicidal compounds, to processes for their preparation, to herbicidal compositions which comprise the novel compounds, and to their use for controlling weeds, in particular in crops of useful plants, or for inhibiting plant growth.

U.S. Pat. Nos. 4,952,235 and 5,068,394 disclose herbicidal (hetero)aryloxynaphthalene derivatives. WO2020/113554 discloses quinoline derivatives.

The present invention relates to heteroaryloxyquinazoline and heteroaryloxyquinoline compounds. Thus, according to the present invention there is provided a compound of Formula (I):

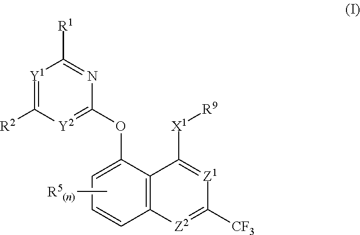

or an agronomically acceptable salt thereof,
wherein
Y¹ is N or CR³;
Y² is N or CR⁴;
with the proviso that Y¹ and Y² are not both N;
R¹ is selected from the group consisting of hydrogen, halogen, C₁-C₃ alkyl and C₁-C₃ haloalkyl;
R² is selected from the group consisting of hydrogen, halogen, C₁-C₃ alkyl, C₁-C₃ alkoxy-, C₁-C₃haloalkoxy- and C₁-C₃ haloalkyl;
R³ is selected from the group consisting of hydrogen, halogen, —CN, nitro, C₁-C₄ alkyl, C₂-C₄ alkenyl-, C₂-C₄ alkynyl-, C₁-C₄ haloalkyl-, C₁-C₄ alkoxy-, C₁-C₄ haloalkoxy- and —S(O)ₙC₁-C₄ alkyl;
R⁴ is selected from the group consisting of hydrogen, halogen, —CN, nitro, C₁-C₄ alkyl, C₂-C₄ alkenyl-, C₂-C₄ alkynyl-, C₁-C₄ haloalkyl-, C₁-C₄ alkoxy-, C₁-C₄ haloalkoxy- and —S(O)ₙC₁-C₄ alkyl;
each R⁵ is independently selected from the group consisting of halogen, —CN, nitro, C₁-C₄ alkyl, C₂-C₄ alkenyl, C₂-C₄ alkynyl, C₁-C₄ haloalkyl, C₁-C₄ alkoxy-, C₁-C₄ haloalkoxy-, —S(O)ₚC₁-C₄ alkyl and —S(O)ₚC₁-C₄ haloalkyl;
X¹ is —(CR¹⁰R¹¹)_{q}—
Z¹ is N or CR⁷;
Z² is N or CR⁸;
R⁷ is selected from the group consisting of hydrogen, C₁-C₄ alkyl, halogen, —CN, nitro, C₂-C₄ alkenyl, C₂-C₄ alkynyl, C₁-C₄ haloalkyl, C₁-C₄ alkoxy-, C₁-C₄ haloalkoxy-, —S(O)ₚC₁-C₄ alkyl and —S(O)ₚC₁-C₄ haloalkyl;
R⁸ is selected from the group consisting of hydrogen, C₁-C₄ alkyl, halogen, —CN, nitro, C₂-C₄ alkenyl, C₂-C₄ alkynyl, C₁-C₄ haloalkyl, C₁-C₄ alkoxy-, C₁-C₄ haloalkoxy-, —S(O)ₚC₁-C₄ alkyl and —S(O)ₚC₁-C₄ haloalkyl;
R⁹ is selected from the group consisting of C₁ haloalkyl-, C₁-C₆ alkyl-C₁ haloalkyl-, hydroxy, C₁-C₃ alkoxy-, —C(O)—R¹⁴, —CN, —C₃-C₆ cycloalkyl, —C(R¹²)═N—O—R¹³, —C(R¹²)═C—N(R¹²)(R¹²), —CR¹⁷ (—CH₂CH₂—), —CR¹⁶ (—CH₂OCH₂—), —CR¹⁶ (—CH₂CH₂OCH₂—), —CR¹⁶ (—CH₂CH₂OCH₂CH₂—), —CR¹⁶(—CH₂CH₂N(R¹⁵)CH₂CH₂—), phenyl and 5- or 6-membered heteroaryl wherein the phenyl and heteroaryl are optionally substituted by 1 or 2 substituents independently selected from the group consisting of halogen, CN, C₁-C₂ alkyl, C₁-C₂ haloalkyl, C₁-C₂ alkoxy- and C₁-C₂ haloalkoxy-, with the proviso that if R¹⁰ and R¹¹ are hydrogen and q=1 then R⁹ is not pyrimidin-2-yl and if R⁹ is C₁ haloalkyl- or C₁-C₆ alkyl-C₁ haloalkyl- then q=0;
R¹⁰ and R¹¹ are independently selected from the group consisting of hydrogen, hydroxy, halogen and C₁-C₃ alkyl;
R¹² is hydrogen or C₁-C₃ alkyl;
R¹³ is selected from the group consisting of hydrogen, C₁-C₆ alkyl, C₁-C₆ haloalkyl, C₂-C₆ alkenyl and —(CH₂) C₃-C₆ cycloalkyl;
R¹⁴ is selected from the group consisting of hydrogen, C₁-C₆ alkyl, —N(R¹²) C₁-C₆ alkyl, phenyl and a 5- or 6-membered heteroaryl wherein the phenyl and heteroaryl are optionally substituted by 1 or 2 substituents independently selected from the group consisting of halogen, CN, C₁-C₂ alkyl, C₁-C₂ haloalkyl, C₁-C₂ alkoxy- and C₁-C₂ haloalkoxy-;
R¹⁵ is C₁C₆ alkoxy- or —C(O) C₁-C₆ alkyl;
R¹⁶ is selected from the group consisting of hydrogen, halogen, CN and C₁-C₂ alkoxy-;
R¹⁷ is selected from the group consisting of halogen, CN and C₁-C₂ alkoxy-;
n=0, 1 or 2;
p=0, 1 or 2 and
q=0, 1, 2, 3 or 4.

C₁-C₄ alkyl- and C₁-C₆ alkyl-includes, for example, methyl (Me, CH₃), ethyl (Et, C₂H₅), n-propyl (n-Pr), isopropyl (i-Pr), n-butyl (n-Bu), isobutyl (i-Bu), sec-butyl and tert-butyl (t-Bu). C₁-C₂alkyl is methyl (Me, CH₃) or ethyl (Et, C₂H₅).

C₁-C₆ alkyl-C₁ haloalkyl-includes, for example, CH₃CHBr—;

Halogen (or halo) includes, for example, fluorine, chlorine, bromine or iodine. The same correspondingly applies to halogen in the context of other definitions, such as haloalkyl.

C₁-C₃ haloalkyl-includes, for example, fluoromethyl, difluoromethyl, trifluoromethyl, chloromethyl, dichloromethyl, trichloromethyl, 2,2,2-trifluoroethyl, 2-fluoroethyl, 2-chloroethyl, pentafluoroethyl, 1,1-difluoro-2,2,2-trichloroethyl, 2,2,3,3-tetrafluoropropyl and 2,2,2-trichloroethyl, heptafluoro-n-propyl and perfluoro-n-hexyl. C₁-C₄ haloalkyl- and C₁-C₂ haloalkyl include, for example, fluoromethyl, difluoromethyl, trifluoromethyl, chloromethyl, dichloromethyl, trichloromethyl, 2,2,2-trifluoroethyl, 2-fluoroethyl, 2-chloroethyl, pentafluoroethyl, or 1,1-difluoro-2,2,2-trichloroethyl.

C₁-C₄ alkoxy and C₁-C₂ alkoxy includes, for example, methoxy and ethoxy.

C₁-C₆haloalkoxy- and C₁-C₄ haloalkoxy-include, for example, fluoromethoxy, difluoromethoxy, trifluoromethoxy, 2,2,2-trifluoroethoxy, 1,1,2,2-tetrafluoroethoxy, 2-fluoroethoxy, 2-chloroethoxy, 2,2-difluoroethoxy or 2,2,2-trichloroethoxy, preferably difluoromethoxy, 2-chloroethoxy or trifluoromethoxy.

C₂-C₄ alkenyl-includes, for example, —CH═CH₂ (vinyl) and —CH₂—CH═CH₂ (allyl).

C₂-C₄ alkynyl-refers to a straight or branched hydrocarbon chain radical group consisting solely of carbon and hydrogen atoms, containing at least one triple bond, having from two to four carbon atoms, and which is attached to the rest of the molecule by a single bond. Examples of C₂-C₄ alkynyl include, but are not limited to, prop-1-ynyl, propargyl (prop-2-ynyl), and but-1-ynyl.

C₁-C₄ alkyl-S-(alkylthio) includes, for example, methylthio, ethylthio, propylthio, isopropylthio, n-butylthio, isobutylthio, sec-butylthio or tert-butylthio, preferably methylthio or ethylthio.

C₁-C₄ alkyl-S(O)-(alkylsulfinyl) includes, for example, methylsulfinyl, ethylsulfinyl, propylsulfinyl, isopropylsulfinyl, n-butylsulfinyl, isobutylsulfinyl, sec-butylsulfinyl or tert-butylsulfinyl, preferably methylsulfinyl or ethylsulfinyl.

C₁-C₄ alkyl-S(O) 2-(alkylsulfonyl) includes, for example, methylsulfonyl, ethylsulfonyl, propylsulfonyl, isopropylsulfonyl, n-butylsulfonyl, isobutylsulfonyl, sec-butylsulfonyl or tert-butylsulfonyl, preferably methylsulfonyl or ethylsulfonyl.

In one embodiment of the present invention there is provided a compound of Formula (I) wherein Y¹ is CR³ and Y² is N (in this embodiment R¹ and R² are preferably hydrogen); or Y¹ is CR³ and Y² is CR⁴ (in this embodiment R¹ and R² are preferably hydrogen); or Y¹ is N and Y² is CR⁴ (in this embodiment R¹ and R² are preferably hydrogen). In a more preferred embodiment of the present invention there is provided a compound of Formula (I) wherein Y¹ is CR³ and Y² is N wherein R³ is C₁-C₄ alkyl (preferably methyl) or halo (preferably chloro), more preferably halo (preferably chloro).

In another embodiment of the present invention there is provided a compound of Formula (I) wherein Z¹ is CR⁷ (preferably CH) and Z² is CR⁸ (preferably CH); or Z¹ is CR⁷ (preferably CH) and Z² is N; or Z¹ is N and Z² is N; or Z¹ is N and Z² is CR⁸. In a more preferred embodiment Z¹ is N and Z² is N.

In another embodiment of the present invention there is provided a compound of Formula (I) wherein n=0. In another embodiment of the present invention there is provided a compound of Formula (I) wherein n=1, wherein R⁵ is selected from the group consisting of fluoro, chloro, bromo and CN.

In another embodiment q=0. In another embodiment q=1. In another embodiment q=2. In another embodiment q=3. In an embodiment where q=1, 2 or 3 then X¹ is preferably CH₂.

In one embodiment of the present invention R⁹ is —C(O)—R¹⁴.

In another embodiment of the present invention R⁹ is selected from the group consisting of —C(R¹²)═N—O—R¹³, —CR¹⁶ (—CH₂OCH₂—), CR¹⁶ (—CH₂CH₂OCH₂—), —CR¹⁶ (—CH₂CH₂OCH₂CH₂—), phenyl and a 5- or 6-membered heteroaryl wherein the phenyl and heteroaryl are optionally substituted by 1 or 2 substituents independently selected from the group consisting of halogen, CN, C₁-C₂ alkyl, C₁-C₂ haloalkyl, C₁-C₂ alkoxy- and C₁-C₂ haloalkoxy-, with the proviso that if R¹⁰ and R¹¹ are hydrogen and q=1 then R⁹ is not pyrimidin-2-yl.

In another embodiment of the present invention there is provided a compound of Formula (I) wherein R⁹ is —C(R¹²)═N—O—R¹³. In this embodiment it is preferred that R¹² is hydrogen or C₁-C₃ alkyl (preferably methyl or ethyl) and R¹³ is C₁-C₆ alkyl, C₁-C₆ haloalkyl, —(CH₂) C₃-C₆ cycloalkyl (preferably cyclpropyl) or C₂-C₆ alkenyl (preferably allyl). In this embodiment X¹ is, for example, CH₂ and q=1 or 2.

In another embodiment of the present invention there is provided a compound of Formula (I) wherein R⁹ is selected from the group consisting of phenyl and 5- or 6-membered heteroaryl wherein the phenyl and heteroaryl are optionally substituted by 1 or 2 substituents independently selected from the group consisting of halogen, CN, C₁-C₂ alkyl, C₁-C₂ haloalkyl, C₁-C₂ alkoxy- and C₁-C₂ haloalkoxy-, with the proviso that if R¹⁰ and R¹¹ are hydrogen and q=1 then R⁹ is not pyrimidin-2-yl. In a preferred embodiment of the present invention the 5- or 6-membered heteroaryl is selected from the group consisting of pyridyl, pyrimidin-5-yl, isoxazolyl and pyrazolyl.

In another embodiment of the present invention there is provided a compound of Formula (I) wherein R⁹ is C₃-C₆ cycloalkyl. In this embodiment X¹ is, for example, CH₂ and q=1, 2 or 3.

Compounds of Formula (I) may contain asymmetric centres and may be present as a single enantiomer, pairs of enantiomers in any proportion or, where more than one asymmetric centre are present, contain diastereoisomers in all possible ratios. Typically one of the enantiomers has enhanced biological activity compared to the other possibilities.

The present invention also provides agronomically acceptable salts of compounds of Formula (I). Salts that the compounds of Formula (I) may form with amines, including primary, secondary and tertiary amines (for example ammonia, dimethylamine and triethylamine), alkali metal and alkaline earth metal bases, transition metals or quaternary ammonium bases are preferred.

The compounds of Formula (I) according to the invention can be used as herbicides by themselves, but they are generally formulated into herbicidal compositions using formulation adjuvants, such as carriers, solvents and surface-active agents (SAA). Thus, the present invention further provides a herbicidal composition comprising a herbicidal compound according to any one of the previous claims and an agriculturally acceptable formulation adjuvant. The composition can be in the form of concentrates which are diluted prior to use, although ready-to-use compositions can also be made. The final dilution is usually made with water, but can be made instead of, or in addition to, water, with, for example, liquid fertilisers, micronutrients, biological organisms, oil or solvents.

The herbicidal compositions generally comprise from 0.1 to 99% by weight, especially from 0.1 to 95% by weight, compounds of Formula I and from 1 to 99.9% by weight of a formulation adjuvant which preferably includes from 0 to 25% by weight of a surface-active substance.

The compositions can be chosen from a number of formulation types. These include an emulsion concentrate (EC), a suspension concentrate (SC), a suspo-emulsion (SE), a capsule suspension (CS), a water dispersible granule (WG), an emulsifiable granule (EG), an emulsion, water in oil (EO), an emulsion, oil in water (EW), a micro-emulsion (ME), an oil dispersion (OD), an oil miscible flowable (OF), an oil miscible liquid (OL), a soluble concentrate (SL), an ultra-low volume suspension (SU), an ultra-low volume liquid (UL), a technical concentrate (TK), a dispersible concentrate (DC), a soluble powder (SP), a wettable powder (WP) and a soluble granule (SG). The formulation type chosen in any instance will depend upon the particular purpose envisaged and the physical, chemical and biological properties of the compound of Formula (I).

Soluble powders (SP) may be prepared by mixing a compound of Formula (I) with one or more water-soluble inorganic salts (such as sodium bicarbonate, sodium carbonate or magnesium sulphate) or one or more water-soluble organic solids (such as a polysaccharide) and, optionally, one or more wetting agents, one or more dispersing agents or a mixture of said agents to improve water dispersibility/solubility. The mixture is then ground to a fine powder. Similar compositions may also be granulated to form water soluble granules (SG).

Wettable powders (WP) may be prepared by mixing a compound of Formula (I) with one or more solid diluents or carriers, one or more wetting agents and, preferably, one or more dispersing agents and, optionally, one or more suspending agents to facilitate the dispersion in liquids. The mixture is then ground to a fine powder. Similar compositions may also be granulated to form water dispersible granules (WG).

Granules (GR) may be formed either by granulating a mixture of a compound of Formula (I) and one or more powdered solid diluents or carriers, or from pre-formed blank granules by absorbing a compound of Formula (I) (or a solution thereof, in a suitable agent) in a porous granular material (such as pumice, attapulgite clays, fuller's earth, kieselguhr, diatomaceous earths or ground corn cobs) or by adsorbing a compound of Formula (I) (or a solution thereof, in a suitable agent) on to a hard core material (such as sands, silicates, mineral carbonates, sulphates or phosphates) and drying if necessary. Agents which are commonly used to aid absorption or adsorption include solvents (such as aliphatic and aromatic petroleum solvents, alcohols, ethers, ketones and esters) and sticking agents (such as polyvinyl acetates, polyvinyl alcohols, dextrins, sugars and vegetable oils). One or more other additives may also be included in granules (for example an emulsifying agent, wetting agent or dispersing agent).

Dispersible Concentrates (DC) may be prepared by dissolving a compound of Formula (I) in water or an organic solvent, such as a ketone, alcohol or glycol ether. These solutions may contain a surface-active agent (for example to improve water dilution or prevent crystallisation in a spray tank).

Emulsifiable concentrates (EC) or oil-in-water emulsions (EW) may be prepared by dissolving a compound of Formula (I) in an organic solvent (optionally containing one or more wetting agents, one or more emulsifying agents or a mixture of said agents). Suitable organic solvents for use in ECs include aromatic hydrocarbons (such as alkylbenzenes or alkylnaphthalenes, exemplified by SOLVESSO 100, SOLVESSO 150 and SOLVESSO 200; SOLVESSO is a Registered Trade Mark), ketones (such as cyclohexanone or methylcyclohexanone) and alcohols (such as benzyl alcohol, furfuryl alcohol or butanol), N-alkylpyrrolidones (such as N-methylpyrrolidone or N-octylpyrrolidone), dimethyl amides of fatty acids (such as C₈-C₁₀ fatty acid dimethylamide) and chlorinated hydrocarbons. An EC product may spontaneously emulsify on addition to water, to produce an emulsion with sufficient stability to allow spray application through appropriate equipment.

Preparation of an EW involves obtaining a compound of Formula (I) either as a liquid (if it is not a liquid at room temperature, it may be melted at a reasonable temperature, typically below 70° C.) or in solution (by dissolving it in an appropriate solvent) and then emulsifying the resultant liquid or solution into water containing one or more SAAs, under high shear, to produce an emulsion. Suitable solvents for use in EWs include vegetable oils, chlorinated hydrocarbons (such as chlorobenzenes), aromatic solvents (such as alkylbenzenes or alkylnaphthalenes) and other appropriate organic solvents which have a low solubility in water.

Microemulsions (ME) may be prepared by mixing water with a blend of one or more solvents with one or more SAAs, to produce spontaneously a thermodynamically stable isotropic liquid formulation. A compound of Formula (I) is present initially in either the water or the solvent/SAA blend. Suitable solvents for use in MEs include those hereinbefore described for use in in ECs or in EWs. An ME may be either an oil-in-water or a water-in-oil system (which system is present may be determined by conductivity measurements) and may be suitable for mixing water-soluble and oil-soluble pesticides in the same formulation. An ME is suitable for dilution into water, either remaining as a microemulsion or forming a conventional oil-in-water emulsion.

Suspension concentrates (SC) may comprise aqueous or non-aqueous suspensions of finely divided insoluble solid particles of a compound of Formula (I). SCs may be prepared by ball or bead milling the solid compound of Formula (I) in a suitable medium, optionally with one or more dispersing agents, to produce a fine particle suspension of the compound. One or more wetting agents may be included in the composition and a suspending agent may be included to reduce the rate at which the particles settle. Alternatively, a compound of Formula (I) may be dry milled and added to water, containing agents hereinbefore described, to produce the desired end product.

Aerosol formulations comprise a compound of Formula (I) and a suitable propellant (for example n-butane). A compound of Formula (I) may also be dissolved or dispersed in a suitable medium (for example water or a water miscible liquid, such as n-propanol) to provide compositions for use in non-pressurised, hand-actuated spray pumps.

Capsule suspensions (CS) may be prepared in a manner similar to the preparation of EW formulations but with an additional polymerisation stage such that an aqueous dispersion of oil droplets is obtained, in which each oil droplet is encapsulated by a polymeric shell and contains a compound of Formula (I) and, optionally, a carrier or diluent therefor. The polymeric shell may be produced by either an interfacial polycondensation reaction or by a coacervation procedure. The compositions may provide for controlled release of the compound of Formula (I) and they may be used for seed treatment. A compound of Formula (I) may also be formulated in a biodegradable polymeric matrix to provide a slow, controlled release of the compound.

The composition may include one or more additives to improve the biological performance of the composition, for example by improving wetting, retention or distribution on surfaces; resistance to rain on treated surfaces; or uptake or mobility of a compound of Formula (I). Such additives include surface active agents (SAAs), spray additives based on oils, for example certain mineral oils or natural plant oils (such as soy bean and rape seed oil), modified plant oils such as methylated rape seed oil (MRSO), and blends of these with other bio-enhancing adjuvants (ingredients which may aid or modify the action of a compound of Formula (I).

Wetting agents, dispersing agents and emulsifying agents may be SAAs of the cationic, anionic, amphoteric or non-ionic type.

Suitable SAAs of the cationic type include quaternary ammonium compounds (for example cetyltrimethyl ammonium bromide), imidazolines and amine salts.

Suitable anionic SAAs include alkali metals salts of fatty acids, salts of aliphatic monoesters of sulphuric acid (for example sodium lauryl sulphate), salts of sulphonated aromatic compounds (for example sodium dodecylbenzenesulphonate, calcium dodecylbenzenesulphonate, butylnaphthalene sulphonate and mixtures of sodium di-isopropyl- and tri-isopropyl-naphthalene sulphonates), ether sulphates, alcohol ether sulphates (for example sodium laureth-3-sulphate), ether carboxylates (for example sodium laureth-3-carboxylate), phosphate esters (products from the reaction between one or more fatty alcohols and phosphoric acid (predominately mono-esters) or phosphorus pentoxide (predominately di-esters), for example the reaction between lauryl alcohol and tetraphosphoric acid; additionally these products may be ethoxylated), sulphosuccinamates, paraffin or olefine sulphonates, taurates, lignosulphonates and phosphates/sulphates of tristyrylphenols.

Suitable SAAs of the amphoteric type include betaines, propionates and glycinates.

Suitable SAAs of the non-ionic type include condensation products of alkylene oxides, such as ethylene oxide, propylene oxide, butylene oxide or mixtures thereof, with fatty alcohols (such as oleyl alcohol or cetyl alcohol) or with alkylphenols (such as octylphenol, nonylphenol or octylcresol); partial esters derived from long chain fatty acids or hexitol anhydrides; condensation products of said partial esters with ethylene oxide; block polymers (comprising ethylene oxide and propylene oxide); alkanolamides; simple esters (for example fatty acid polyethylene glycol esters); amine oxides (for example lauryl dimethyl amine oxide); lecithins and sorbitans and esters thereof, alkyl polyglycosides and tristyrylphenols.

Suitable suspending agents include hydrophilic colloids (such as polysaccharides, polyvinylpyrrolidone or sodium carboxymethylcellulose) and swelling clays (such as bentonite or attapulgite).

The compounds of present invention can also be used in mixture with one or more additional herbicides and/or plant growth regulators. Examples of such additional herbicides or plant growth regulators include acetochlor, acifluorfen (including acifluorfen-sodium), aclonifen, ametryn, amicarbazone, aminopyralid, aminotriazole, atrazine, beflubutamid-M, benquitrione, bensulfuron (including bensulfuron-methyl), bentazone, bicyclopyrone, bilanafos, bipyrazone, bispyribac-sodium, bixlozone, bromacil, bromoxynil, butachlor, butafenacil, carfentrazone (including carfentrazone-ethyl), cloransulam (including cloransulam-methyl), chlorimuron (including chlorimuron-ethyl), chlorotoluron, chlorsulfuron, cinmethylin, clacyfos, clethodim, clodinafop (including clodinafop-propargyl), clomazone, clopyralid, cyclopyranil, cyclopyrimorate, cyclosulfamuron, cyhalofop (including cyhalofop-butyl), 2,4-D (including the choline salt and 2-ethylhexyl ester thereof), 2,4-DB, desmedipham, dicamba (including the aluminium, aminopropyl, bis-aminopropylmethyl, choline, dichloroprop, diglycolamine, dimethylamine, dimethylammonium, potassium and sodium salts thereof) diclosulam, diflufenican, diflufenzopyr, dimethachlor, dimethenamid-P, dioxopyritrione, diquat dibromide, diuron, epyrifenacil, ethalfluralin, ethofumesate, fenoxaprop (including fenoxaprop-P-ethyl), fenoxasulfone, fenpyrazone, fenquinotrione, fentrazamide, flazasulfuron, florasulam, florpyrauxifen (including florpyrauxifen-benzyl), fluazifop (including fluazifop-P-butyl), flucarbazone (including flucarbazone-sodium), flufenacet, flumetsulam, flumioxazin, fluometuron, fomesafen flupyrsulfuron (including flupyrsulfuron-methyl-sodium), fluroxypyr (including fluroxypyr-meptyl), fomesafen, foramsulfuron, glufosinate (including L-glufosinate and the ammonium salts of both), glyphosate (including the diammonium, isopropylammonium and potassium salts thereof), halauxifen (including halauxifen-methyl), haloxyfop (including haloxyfop-methyl), hexazinone, hydantocidin, imazamox (including R-imazamox), imazapic, imazapyr, imazethapyr, indaziflam, iodosulfuron (including iodosulfuron-methyl-sodium), iofensulfuron (including iofensulfuron-sodium), ioxynil, isoproturon, isoxaflutole, lancotrione, MCPA, MCPB, mecoprop-P, mesosulfuron (including mesosulfuron-methyl), mesotrione, metamitron, metazachlor, methiozolin, metolachlor, metosulam, metribuzin, metsulfuron, napropamide, nicosulfuron, norflurazon, oxadiazon, oxasulfuron, oxyfluorfen, paraquat dichloride, pendimethalin, penoxsulam, phenmedipham, picloram, pinoxaden, pretilachlor, primisulfuron-methyl, prometryne, propanil, propaquizafop, propyrisulfuron, propyzamide, prosulfocarb, prosulfuron, pyraclonil, pyraflufen (including pyraflufen-ethyl), pyrasulfotole, pyridate, pyriftalid, pyrimisulfan, pyroxasulfone, pyroxsulam, quinclorac, quinmerac, quizalofop (including quizalofop-P-ethyl and quizalofop-P-tefuryl), rimisoxafen, rimsulfuron, saflufenacil, sethoxydim, simazine, S-metalochlor, sulfentrazone, sulfosulfuron, tebuthiuron, tefuryltrione, tembotrione, terbuthylazine, terbutryn, tetflupyrolimet, thiencarbazone, thifensulfuron, tiafenacil, tolpyralate, topramezone, tralkoxydim, triafamone, triallate, triasulfuron, tribenuron (including tribenuron-methyl), triclopyr, trifloxysulfuron (including trifloxysulfuron-sodium), trifludimoxazin, trifluralin, triflusulfuron, tripyrasulfone, 3-(2-chloro-4-fluoro-5-(3-methyl-2,6-dioxo-4-trifluoromethyl-3,6-dihydropyrimidin-1 (2H)-yl)phenyl)-5-methyl-4,5-dihydroisoxazole-5-carboxylic acid ethyl ester, 4-hydroxy-1-methoxy-5-methyl-3-[4-(trifluoromethyl)-2-pyridyl]imidazolidin-2-one, 4-hydroxy-1,5-dimethyl-3-[4-(trifluoromethyl)-2-pyridyl]imidazolidin-2-one, 5-ethoxy-4-hydroxy-1-methyl-3-[4-(trifluoromethyl)-2-pyridyl]imidazolidin-2-one, 4-hydroxy-1-methyl-3-[4-(trifluoromethyl)-2-pyridyl]imidazolidin-2-one, 4-hydroxy-1,5-dimethyl-3-[1-methyl-5-(trifluoromethyl) pyrazol-3-yl]imidazolidin-2-one, (4R) 1-(5-tert-butylisoxazol-3-yl)-4-ethoxy-5-hydroxy-3-methyl-imidazolidin-2-one, 4-amino-3-chloro-5-fluoro-6-(7-fluoro-1H-indol-6-yl)pyridine-2-carboxylic acid (including agrochemically acceptable esters thereof, for example, methyl 4-amino-3-chloro-5-fluoro-6-(7-fluoro-1H-indol-6-yl)pyridine-2-carboxylate, prop-2-ynyl 4-amino-3-chloro-5-fluoro-6-(7-fluoro-1H-indol-6-yl)pyridine-2-carboxylate and cyanomethyl 4-amino-3-chloro-5-fluoro-6-(7-fluoro-1H-indol-6-yl)pyridine-2-carboxylate), 3-ethylsulfanyl-N-(1,3,4-oxadiazol-2-yl)-5-(trifluoromethyl)-[1,2,4]triazolo[4,3-a]pyridine-8-carboxamide, 3-(isopropylsulfanylmethyl)-N-(5-methyl-1,3,4-oxadiazol-2-yl)-5-(trifluoromethyl)-[1,2,4]triazolo[4,3-a]pyridine-8-carboxamide, 3-(isopropylsulfonylmethyl)-N-(5-methyl-1,3,4-oxadiazol-2-yl)-5-(trifluoromethyl)-[1,2,4]triazolo[4,3-a]pyridine-8-carboxamide, 3-(ethylsulfonylmethyl)-N-(5-methyl-1,3,4-oxadiazol-2-yl)-5-(trifluoromethyl)-[1,2,4]triazolo[4,3-a]pyridine-8-carboxamide, ethyl-2-[[3-[[3-chloro-5-fluoro-6-[3-methyl-2,6-dioxo-4-(trifluoromethyl)pyrimidin-1-yl]-2-pyridyl]oxy]acetate,6-chloro-4-(2,7-dimethyl-1-naphthyl)-5-hydroxy-2-methyl-pyridazin-3-one, tetrahydrofuran-2-ylmethyl (2R)-2-[(4-amino-3,5-dichloro-6-fluoro-2-pyridyl)oxy]propanoate, tetrahydrofuran-2-ylmethyl (2R)-2-[(4-amino-3,5-dichloro-6-fluoro-2-pyridyl)oxy]propanoate, tetrahydrofuran-2-ylmethyl 2-[(4-amino-3,5-dichloro-6-fluoro-2-pyridyl)oxy]propanoate, 2-[(4-amino-3,5-dichloro-6-fluoro-2-pyridyl)oxy]propanoic acid, 2-fluoro-N-(5-methyl-1,3,4-oxadiazol-2-yl)-3-[(R)-propylsulfinyl]-4-(trifluoromethyl)benzamide, 2-fluoro-N-(5-methyl-1,3,4-oxadiazol-2-yl)-3-propylsulfinyl-4-(trifluoromethyl)benzamide, (2-fluorophenyl)methyl 6-amino-5-chloro-2-(4-chloro-2-fluoro-3-methoxy-phenyl)pyrimidine-4-carboxylate and 6-amino-5-chloro-2-(4-chloro-2-fluoro-3-methoxy-phenyl)pyrimidine-4-carboxylic acid.

The mixing partners of the compound of Formula (I) may also be in the form of esters or salts, as mentioned e.g. in The Pesticide Manual, Sixteenth Edition, British Crop Protection Council, 2012.

The compound of Formula (I) can also be used in mixtures with other agrochemicals such as fungicides, nematicides or insecticides, examples of which are given in The Pesticide Manual.

The mixing ratio of the compound of Formula (I) to the mixing partner is preferably from 1:100 to 1000:1.

The mixtures can advantageously be used in the above-mentioned formulations (in which case “active ingredient” relates to the respective mixture of compound of Formula (I) with the mixing partner).

The compounds or mixtures of the present invention can also be used in combination with one or more herbicide safeners. Examples of such safeners include benoxacor, cloquintocet (including cloquintocet-mexyl), cyprosulfamide, dichlormid, fenchlorazole (including fenchlorazole-ethyl), fenclorim, fluxofenim, furilazole, isoxadifen (including isoxadifen-ethyl), mefenpyr (including mefenpyr-diethyl), metcamifen and oxabetrinil.

Particularly preferred are mixtures of a compound of Formula (I) with cyprosulfamide, isoxadifen-ethyl, cloquintocet-mexyl and/or metcamifen.

The safeners of the compound of Formula (I) may also be in the form of esters or salts, as mentioned e.g. in The Pesticide Manual, 16th Edition (BCPC), 2012. The reference to cloquintocet-mexyl also applies to a lithium, sodium, potassium, calcium, magnesium, aluminium, iron, ammonium, quaternary ammonium, sulfonium or phosphonium salt thereof as disclosed in WO 02/34048.

Preferably the mixing ratio of compound of Formula (I) to safener is from 100:1 to 1:10, especially from 20:1 to 1:1.

The present invention still further provides a method of controlling weeds at a locus said method comprising application to the locus of a weed controlling amount of a composition comprising a compound of Formula (I). Moreover, the present invention may further provide a method of selectively controlling weeds at a locus comprising crop plants and weeds, wherein the method comprises application to the locus of a weed controlling amount of a composition according to the present invention. ‘Controlling’ means killing, reducing or retarding growth or preventing or reducing germination. It is noted that the compounds of the present invention show a much-improved selectivity compared to know, structurally similar compounds. Generally the plants to be controlled are unwanted plants (weeds). ‘Locus’ means the area in which the plants are growing or will grow. The application may be applied to the locus pre-emergence and/or postemergence of the crop plant. Some crop plants may be inherently tolerant to herbicidal effects of compounds of Formula (I). Preferred crop plants include maize, wheat, barley and rice.

The rates of application of compounds of Formula I may vary within wide limits and depend on the nature of the soil, the method of application (pre- or post-emergence; seed dressing; application to the seed furrow; no tillage application etc.), the crop plant, the weed(s) to be controlled, the prevailing climatic conditions, and other factors governed by the method of application, the time of application and the target crop. The compounds of Formula I according to the invention are generally applied at a rate of from 10 to 2500 g/ha, especially from 25 to 1000 g/ha, more especially from 25 to 250 g/ha.

The application is generally made by spraying the composition, typically by tractor mounted sprayer for large areas, but other methods such as dusting (for powders), drip or drench can also be used.

Crop plants are to be understood as also including those crop plants which have been rendered tolerant to other herbicides or classes of herbicides (e.g. ALS-, GS-, EPSPS-, PPO—, HPPD-, -PDS and ACCase-inhibitors) by conventional methods of breeding or by genetic engineering. An example of a crop that has been rendered tolerant to imidazolinones, e.g. imazamox, by conventional methods of breeding is Clearfield® summer rape (canola). Examples of crops that have been rendered tolerant to herbicides by genetic engineering methods include e.g. glyphosate- and glufosinate-resistant maize varieties commercially available under the trade names RoundupReady® and LibertyLink®. The compounds of the present invention may also be used in conjunction with plants disclosed in WO2020/236790.

Crop plants are also to be understood as being those which have been rendered resistant to harmful insects by genetic engineering methods, for example Bt maize (resistant to European corn borer), Bt cotton (resistant to cotton boll weevil) and also Bt potatoes (resistant to Colorado beetle). Examples of Bt maize are the Bt 176 maize hybrids of NK® (Syngenta Seeds). The Bt toxin is a protein that is formed naturally by Bacillus thuringiensis soil bacteria. Examples of toxins, or transgenic plants able to synthesise such toxins, are described in EP-A-451 878, EP-A-374 753, WO 93/07278, WO 95/34656, WO 03/052073 and EP-A-427 529. Examples of transgenic plants comprising one or more genes that code for an insecticidal resistance and express one or more toxins are KnockOut® (maize), Yield Gard® (maize), NuCOTIN33B® (cotton), Bollgard® (cotton), NewLeaf® (potatoes), NatureGard® and Protexcta®. Plant crops or seed material thereof can be both resistant to herbicides and, at the same time, resistant to insect feeding (“stacked” transgenic events). For example, seed can have the ability to express an insecticidal Cry3 protein while at the same time being tolerant to glyphosate.

Crop plants are also to be understood to include those which are obtained by conventional methods of breeding or genetic engineering and contain so-called output traits (e.g. improved storage stability, higher nutritional value and improved flavour).

The compositions can be used to control unwanted plants (collectively, ‘weeds’). The weeds to be controlled may be both monocotyledonous species, for example Agrostis, Alopecurus, Avena, Brachiaria, Bromus, Cenchrus, Cyperus, Digitaria, Echinochloa, Eleusine, Lolium, Monochoria, Rottboellia, Sagittaria, Scirpus, Setaria and Sorghum, and dicotyledonous species, for example Abutilon, Amaranthus, Ambrosia, Chenopodium, Chrysanthemum, Conyza, Galium, Ipomoea, Nasturtium, Sida, Sinapis, Solanum, Stellaria, Veronica, Viola and Xanthium.

In a further aspect of the present invention there is provided the use of a compound of Formula (I) as defined herein as a herbicide.

Processes for preparation of compounds, e.g. a compound of formula (I) (which optionally can be an agrochemically acceptable salt thereof), are now described, and form further aspects of the present invention.

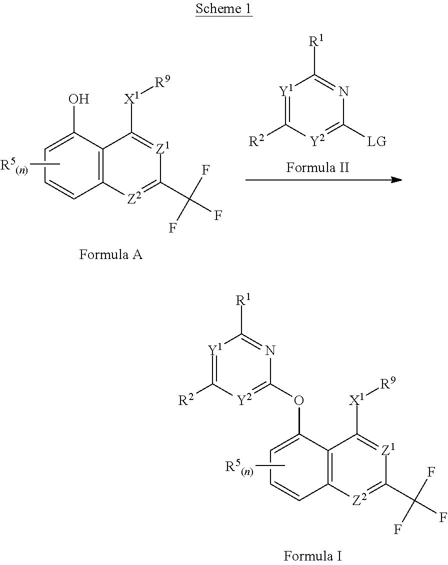

A compound of Formula I may be prepared from a compound of Formula A by reaction with a compound of Formula II (where LG represents a suitable leaving group such as F, Cl, Br or SO₂Me) in the presence of a suitable base and in a suitable solvent. Suitable bases may include NaH, K₂CO₃, Cs₂CO₃. Suitable solvents may include THF, MeCN, isopropanol or DMF. Compounds of Formula II are commercially available or may be prepared by known methods.

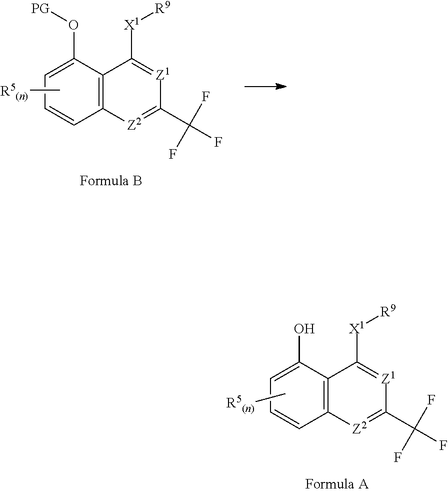

A compound of Formula A may be prepared from a compound of Formula B (where PG represents a suitable protecting group such as Me or Tf) by a deprotection reaction in a suitable solvent. Suitable deprotecting conditions may include BBr₃ or dodecanethiol/LiO^{t}Bu or pyridine hydrochloride (for PG=Me) or K₂CO₃ (for PG=Tf) or H₂/Pd—C(for PG=benzyl) Suitable solvents may include DCM, DCE, DMF, CH₃CN, MeOH or EtOH.

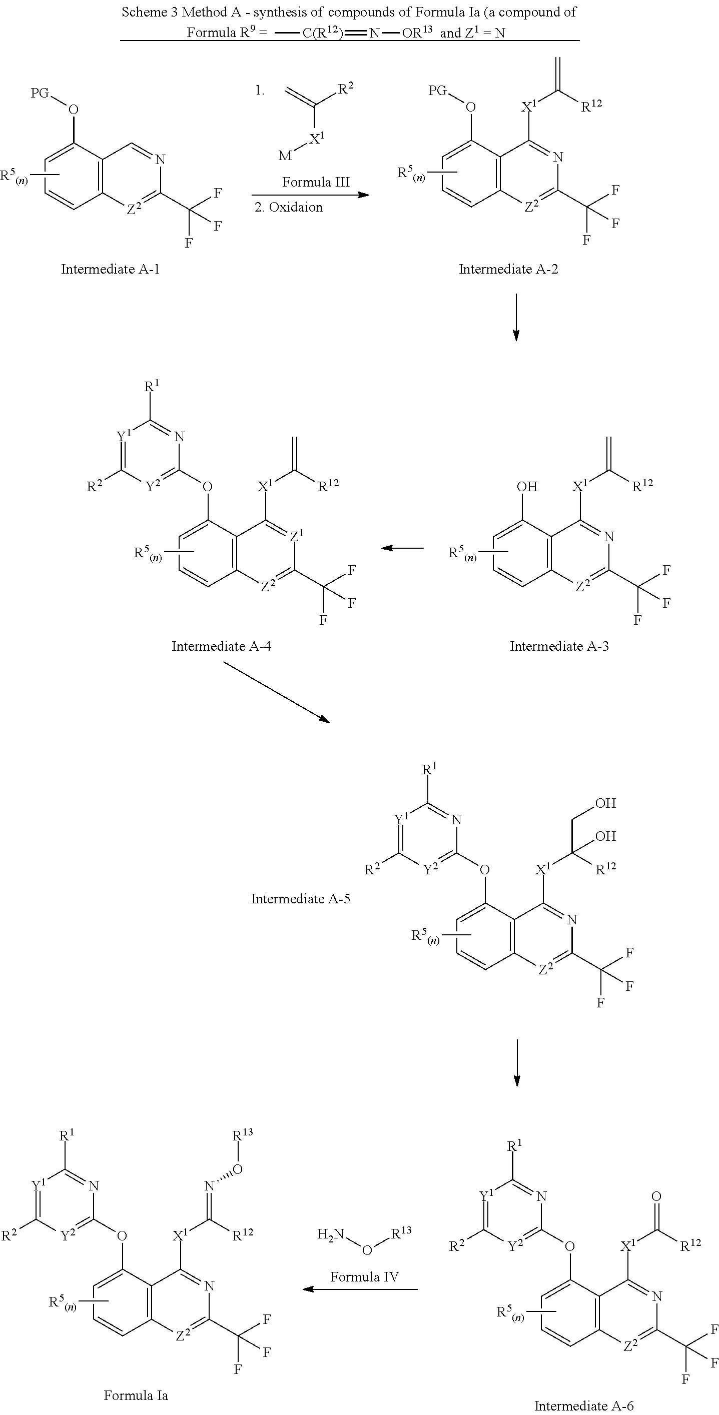

A compound of Formula Intermediate A-2 may be prepared from a compound of Formula Intermediate A-1 in a two-step process. The first step involves reaction with a compound of Formula III (where M is a suitable organometallic such as Li or MgHal), optionally in the presence of a suitable catalyst and in a suitable solvent. Suitable catalysts may include lanthanum (III) chloride bis(lithium chloride) complex. Suitable solvents may include THF. The second step involves reaction with a suitable oxidising agent in a suitable solvent. Suitable oxidising agents may include 3-dichloro-5,6-dicyano-1,4-benzoquinone or potassium ferricyanide. Suitable solvents may include THF or Et₂O/water. Compounds of Formula Intermediate A-1 and of Formula III are commercially available or may be prepared by known methods. A compound of Formula Intermediate A-3 may be prepared from a compound of Formula Intermediate A-2 by a deprotection reaction as described in General Method 2 above.

A compound of Formula Intermediate A-4 may be prepared from a compound of Formula Intermediate A-3 by a method as described in General Method 1 above.

A compound of Formula Intermediate A-5 may be prepared from a compound of Formula Intermediate A-4 by dihydroxylation in the presence of a suitable reagent and in a suitable solvent. Suitable reagents may include potassium osmate(VI) dihydrate. Suitable solvents may include THF/water.

A compound of Formula Intermediate A-6 may be prepared from a compound of Formula Intermediate A-5 by reaction with a suitable oxidising agent in a suitable solvent. Suitable oxidising agents may include sodium periodate. Suitable solvents may include THF/water.

A compound of Formula Ia may be prepared from a compound of Formula Intermediate A-6 by reaction with a compound of Formula IV (or a salt thereof) in the presence of an appropriate base in an appropriate solvent. Suitable bases may include triethyl amine. Suitable solvents may include MeCN.

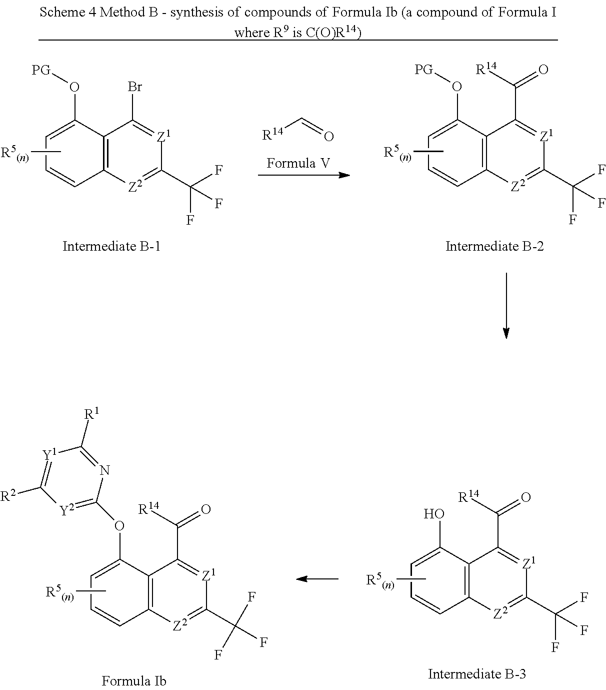

A compound of Formula Intermediate B-2 may be prepared from a compound of formula Intermediate B-1 by reaction with a compound of Formula V in the presence of an appropriate catalyst and an appropriate base in an appropriate solvent. Suitable catalysts may include 1,3-dimethylimidazolium iodide. Suitable bases may include sodium hydride. Suitable solvents may include 1,4 dioxane.

A compound of Formula Intermediate B-3 may be prepared from a compound of Formula Intermediate B-2 by a deprotection reaction as described in General Method 2 above.

A compound of Formula Ib may be prepared from a compound of Formula Intermediate B-3 by a method as described in General Method 1 above.

Method C-synthesis of compounds of Formula Ic (a compound of Formula I where q=0, R⁹=—C(R¹²)═N—OR¹³ and Z¹=N).

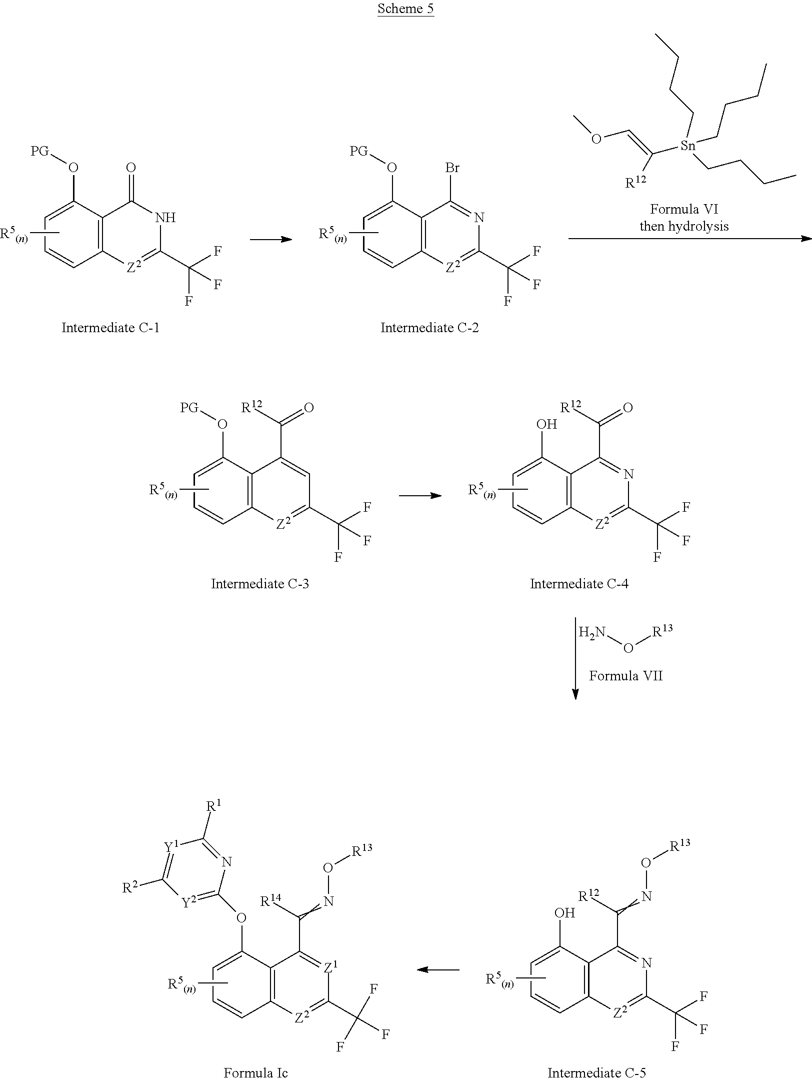

A compound of formula Intermediate C-2 may be prepared from a compound of Formula Intermediate C-1 by reaction with a suitable brominating agent in a suitable solvent. Suitable reagents may include POBr₃. Suitable solvents may include toluene. Compounds of Formula Intermediate C-1 may be prepared by known methods. A compound of formula Intermediate C-3 may be prepared from a compound of Formula Intermediate C-2 in a 2 step procedure by reaction with a compound of Formula VI in the presence of an appropriate metal catalyst and in an appropriate solvent. Suitable catalysts may include tetrakis(triphenylphosphine)palladium(0). Suitable solvents may include toluene. In a subsequent step the resulting intermediate is converted to compounds of Formula Intermediate C-3 by reaction with an appropriate acid in an appropriate solvent. Suitable acids may include hydrochloric acid. Suitable solvents may include water.

A compound of Formula Intermediate C-4 may be prepared from a compound of Formula Intermediate C-3 by a deprotection reaction described in General Method 2 above.

A compound of Formula Intermediate C-5 may be prepared from a compound of Formula Intermediate C-4 by reaction with a compound of Formula VII (or a salt thereof) in the presence of an appropriate base in an appropriate solvent. Suitable bases may include triethylamine. Suitable solvents may include MeCN.

A compound of Formula Ic may be prepared from a compound of Formula Intermediate C-5 by a method described in General Method 1 above.

Method D-synthesis of compounds of Formula Id (a compound of Formula I where X¹=—CHR¹⁰— and R⁹=—CH═N—OR¹³.

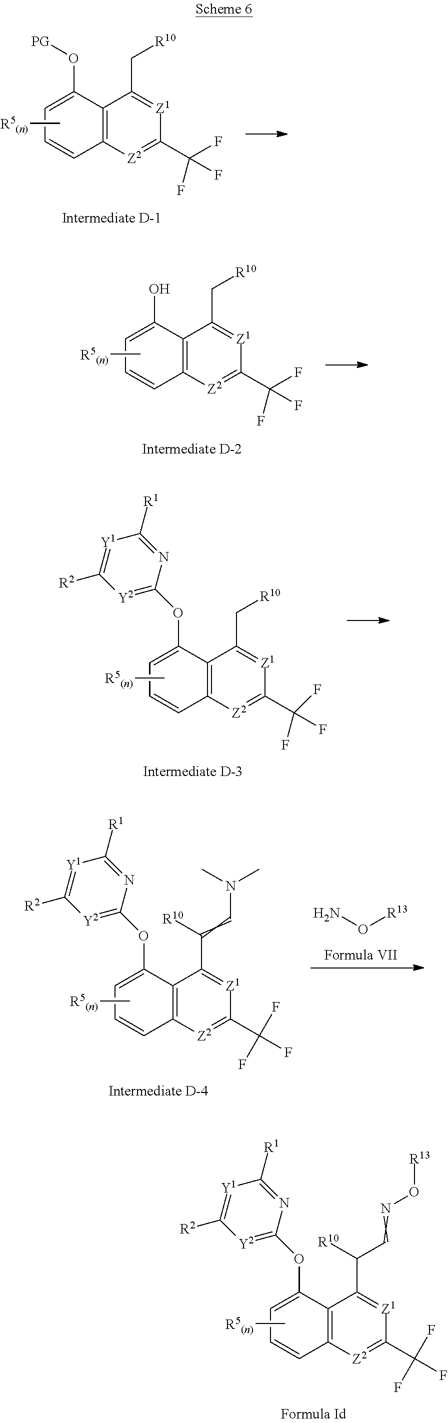

A compound of Formula Intermediate D-2 may be prepared from a compound of Formula Intermediate D-1 by a method described in General Method 2 above.

A compound of Formula Intermediate D-3 may be prepared from a compound of Formula Intermediate D-2 by a method described in General Method 1 above.

A compound of Formula Intermediate D-4 may be prepared from a compound of Formula Intermediate D-3 by reaction with N, N-dimethylformamide dimethyl acetal in an appropriate solvent. Suitable solvents may include toluene.

A compound of Formula Id may be prepared from a compound of Formula Intermediate D-4 by reaction with a compound of Formula VII (or a salt thereof) in the presence of an appropriate base in an appropriate solvent. Suitable bases may include triethylamine. Suitable solvents may include MeCN.

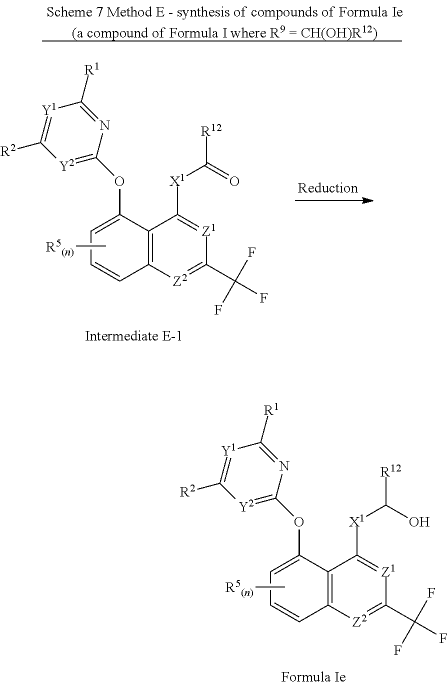

A compound of Formula Ie may be prepared from a compound of Formula Intermediate E-1 by reaction with a reducing agent in an appropriate solvent. Suitable reducing agents include sodium borohydride. Suitable solvents may include THF. Compounds of Formula Intermediate E-1 may be prepared by methods described in Method A above.

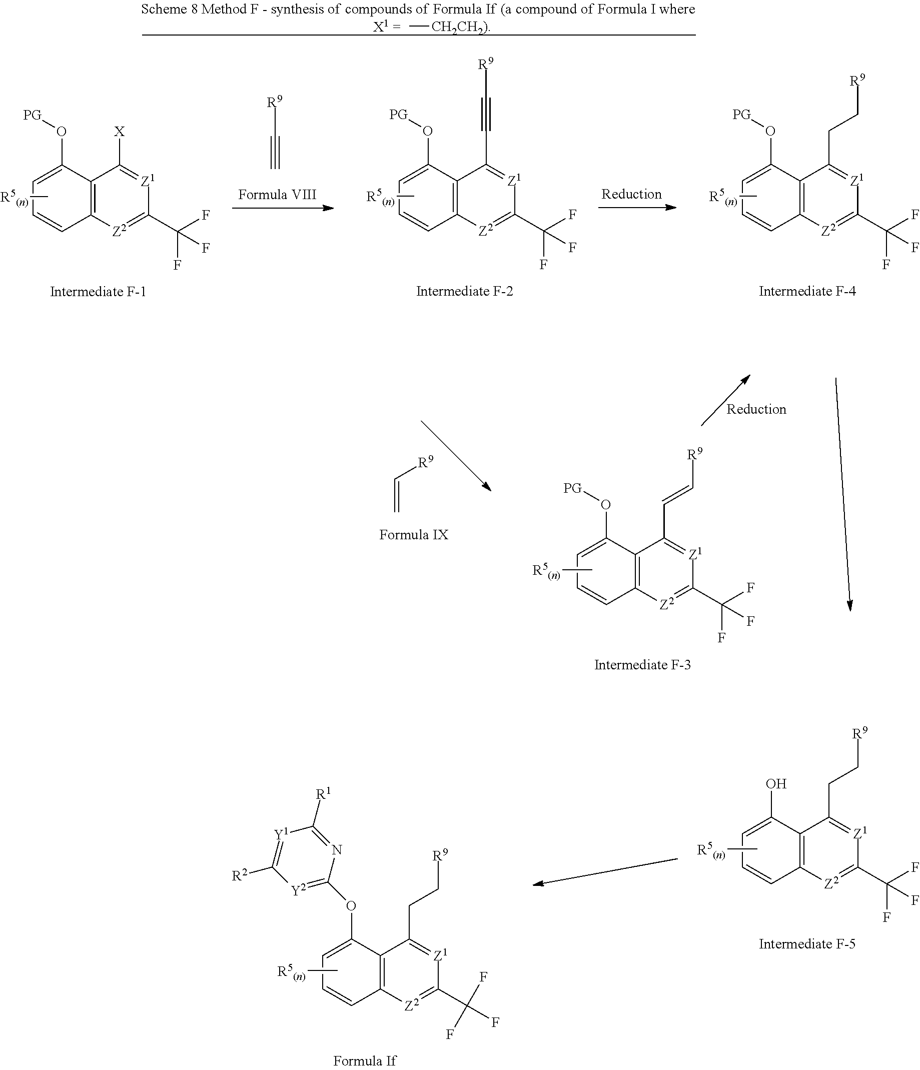

A compound of Formula Intermediate F-2 may be prepared from a compound of Formula Intermediate F-1 (where X is a suitable halogen such as Cl, Br or I) by reaction with a compound of Formula VIII in the presence of an appropriate catalyst system, an appropriate base and in an appropriate solvent. Suitable catalyst systems may include tetrakis (triphenylphosphine)palladium(0) or dichlorobis(triphenylphosphine)palladium(II) and copper(I) iodide. Suitable bases may include Cs₂CO₃ or triethylamine. Suitable solvents may include DMF and THF. Compounds of formula Intermediate F-1 and of Formula VIII are commercially available or may be prepared by known methods.

A compound of Formula Intermediate F-3 may be prepared from a compound of Formula Intermediate F-1 by reaction with a compound of Formula IX in the presence of an appropriate catalyst, an appropriate base and in an appropriate solvent.

Suitable catalysts may include chloro[(tri-tert-butylphosphine)-2-(2-aminobiphenyl)] palladium(II). Suitable bases may include N,N-diisopropylethylamine. Suitable solvents may include toluene. Compounds of Formula IX are commercially available or may be prepared by known methods.

A compound of Formula Intermediate F-4 may be prepared from a compound of Formula Intermediate F-2 or of Formula Intermediate F-3 by reduction in the presence of hydrogen gas, an appropriate catalyst and in an appropriate solvent. Suitable catalysts may include palladium on carbon. Suitable solvents may include methanol.

A compound of Formula Intermediate F-5 may be prepared from a compound of Formula F-4 by a deprotection described in General Method 2 above.

A compound of Formula If may be prepared from a compound of Formula F-5 by a method described in General Method 1 above.

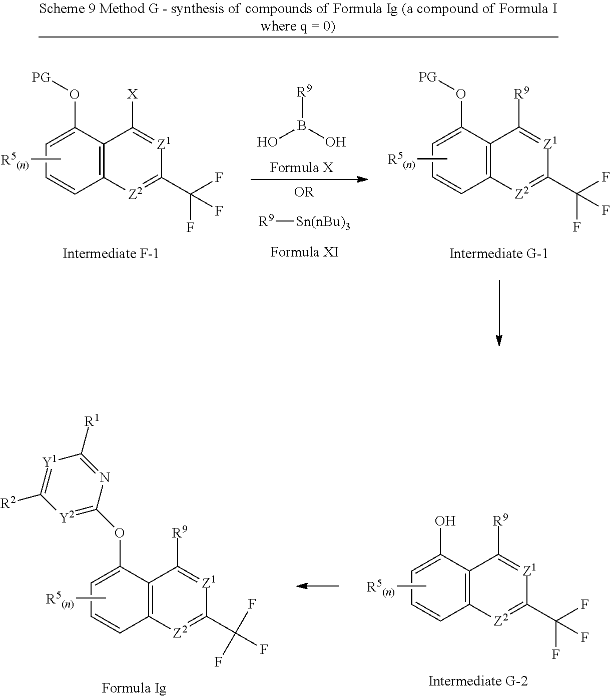

A compound of Formula Intermediate G-1 may be prepared from a compound of Formula Intermediate F-1 by reaction with a compound of Formula X in the presence of an appropriate catalyst or catalyst/ligand combination, an appropriate base and in an appropriate solvent. Suitable catalysts may include [1,1′bis(diphenylphosphino)ferrocene] dichloropalladium(II) or palladium diacetate in combination with tricyclohexylphoshine. Suitable bases may include Cs₂CO₃ or K₃PO₄. Suitable solvents may include 2-MeTHF or toluene. Compounds of Formula X are commercially available or may be prepared by known methods.

Alternatively, compound of Formula Intermediate G-1 may be prepared from a compound of Formula Intermediate F-1 by reaction with a compound of Formula XI in the presence of appropriate catalysts and in an appropriate solvent. Suitable catalysts may include tetrakis(triphenylphosphine)palladium (0) and copper (I) iodide.

Suitable solvents may include toluene. Compounds of Formula XI are commercially available or may be prepared by known methods.

A compound of Formula Intermediate G-2 may be prepared from a compound of Formula G-1 by a deprotection reaction as described in General Method 2 above.

A compound of Formula Ig may be prepared from a compound of Formula Intermediate G-2 by a method described in General Method 1 above.

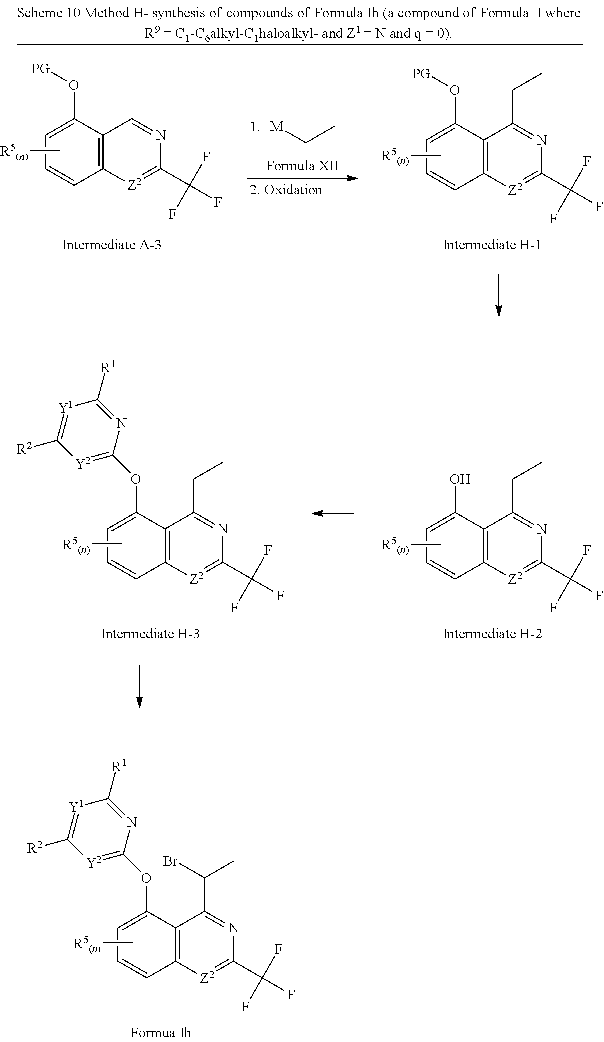

A compound of Formula Intermediate H-1 may be prepared from a compound of Formula Intermediate A-3 in a two-step process. The first step involves reaction with a compound of Formula XII (where M is a suitable organometallic such as MgHal), optionally in the presence of a suitable catalyst and in a suitable solvent. Suitable catalysts may include lanthanum (III) chloride bis(lithium chloride) complex. Suitable solvents may include THF. The second step involves reaction with a suitable oxidising agent in a suitable solvent. Suitable oxidising agents may include 3-dichloro-5,6-dicyano-1,4-benzoquinone or potassium ferricyanide. Suitable solvents may include THF or Et₂O/water. Compounds of Formula A-3 and of Formula XII are commercially available or may be prepared by known methods.

A compound of Formula Intermediate H-2 may be prepared from a compound of Formula H-1 by a deprotection reaction as described in General Method 2 above.

A compound of Formula Ih may be prepared from a compound of formula Intermediate H-3 by reaction with a brominating agent in the presence of an appropriate radical initiator and in a suitable solvent. Suitable brominating agents may include N-bromosuccinimide. A suitable radical initiator is azobisisobutyronitrile. Suitable solvents may include CCl₄.

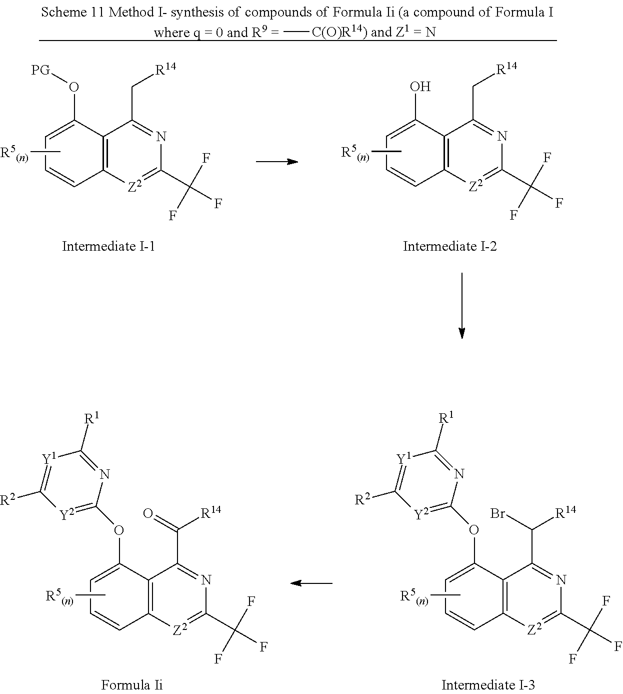

A compound of Formula Intermediate I-2 may be prepared from a compound of Formula Intermediate I-1 by a deprotection method as described in General Method 2 above.

A compound of Formula Intermediate 1-3 may be prepared from a compound of Formula Intermediate I-2 by the method described in General Method 1 above.

A compound of Formula Ii may be prepared from a compound of Formula Intermediate 1-3 reaction with a suitable oxidising agent in an appropriate solvent. Suitable oxidising agents may include N-methyl morpholine-N-oxide. Suitable solvents may include MeCN.

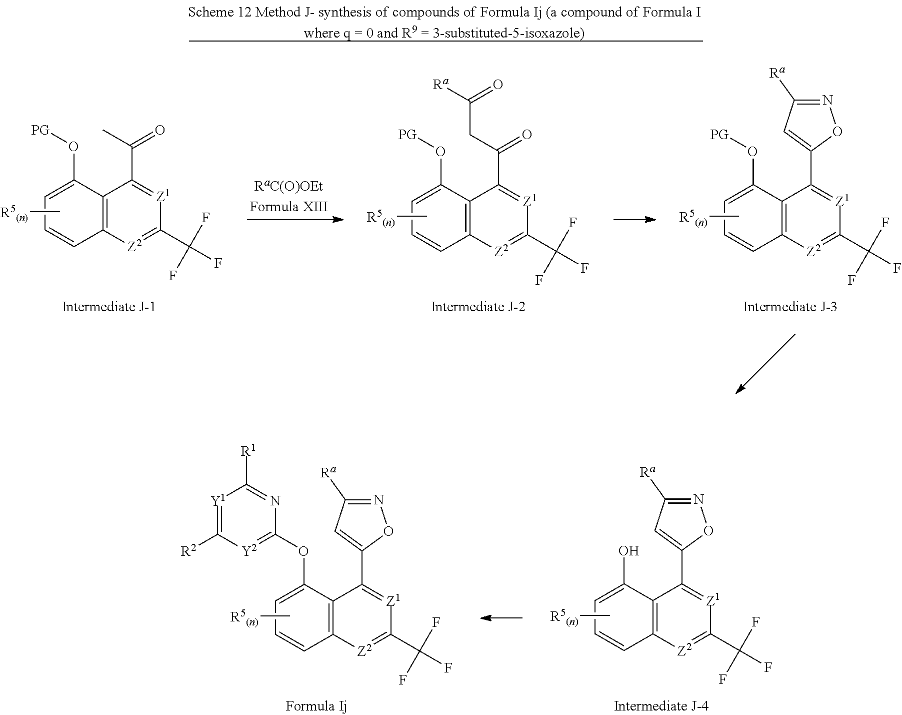

A compound of Formula Intermediate J-2 (where R^{a}=C₁-C₂ alkyl or C₁-C₂ haloalkyl) may be prepared from a compound of Formula Intermediate J-1 by reaction with compounds of Formula XIII in the presence of an appropriate base and in an appropriate solvent. Suitable bases include sodium hydride. Suitable solvents may include THF. Compounds of Formula XIII are commercially available or may be prepared by known methods.

A compound of Formula Intermediate J-3 may be prepared from compounds of formula Intermediate J-2 by reaction with hydroxylamine hydrochloride in an appropriate solvent. Suitable solvents may include ethanol.

A compound of Formula Intermediate J-4 may be prepared from a compound of Formula Intermediate J-3 by a deprotection reaction as described in General Method 2 above.

A compound of Formula Ij may be prepared from a compound of Formula Intermediate J-4 by a methods as described in General Method 1 above.

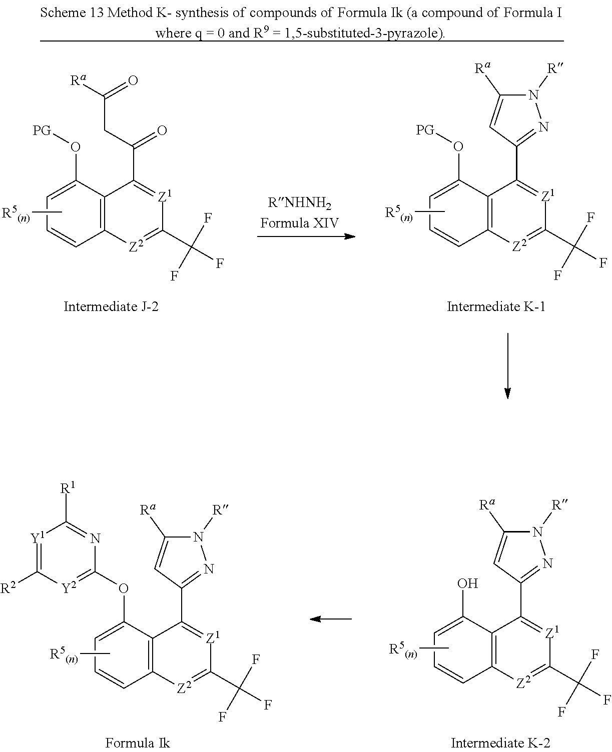

A compound of Formula Intermediate K-1 (where R^{a}=C₁-C₂ alkyl or C₁-C₂ haloalkyl and R″=C₁-C₂ alkyl) may be prepared from a compound of Formula Intermediate J-2 by reaction with a compound of Formula XIV in the presence of appropriate base and an appropriate solvent. Suitable bases include pyridine. Suitable solvents may include MeCN.

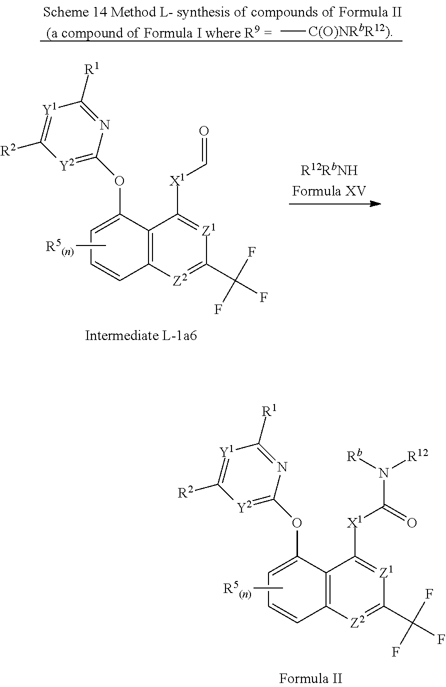

A compound of Formula II (where R^{b}=C₁-C₆ alkyl) may be prepared from a compound of Formula Intermediate L-1a6 (a compound of Formula Intermediate A-6 where R¹²=H) by reaction with a compound of Formula XV in the presence of N-hydroxysuccinimide and (diacetoxyiodo)benzene in an appropriate solvent. Suitable solvents may include MeCN.

The following non-limiting examples provide specific synthesis methods for representative compounds of the present invention, as referred to in the Table below.

LCMS spectra were recorded on a ACQUITY Mass Spectrometer from Waters Corporations (SQD or SQDII Single quadrupole mass spectrometer) equipped with an electrospray source (Polarity: positive or negative ions, Capillary: 3.0 kV, Cone: 30V, Extractor: 3.00 V, Source Temperature: 150° C., Desolvation Temperature: 400° C., Cone Gas Flow: 60 L/hr, Desolvation Gas Flow: 700 L/hr, Mass range: 140 to 800 Da) and an ACQUITY UPLC from Waters Corporations with solvent degasser, binary pump, heated column compartment and diode-array detector. Column: Waters UPLC HSS T3, 1.8 μm, 30×2.1 mm, Temp: 60° C., DAD Wavelength range (nm): 210 to 400, Solvent Gradient: A=Water/Methanol 9:1+0.1% formic acid, B=Acetonitrile+0.1% formic acid, gradient: 0-100% B in 2.5 min; Flow (ml/min) 0.75.

### Example 1: (E)-3-[5-(5-chloropyrimidin-2-yl)oxy-2-(trifluoromethyl)quinazolin-4-yl]-N-methoxy-propan-1-imine and (Z)-3-[5-(5-chloropyrimidin-2-yl)oxy-2-(trifluoromethyl)quinazolin-4-yl]-N-methoxy-propan-1-imine (Compounds 1.022 & 1.025)

### Step1: Synthesis of 4-but-3-enyl-5-methoxy-2-(trifluoromethyl)quinazoline

To a solution of Lanthanum (III) trichloride bis (lithium chloride) complex (20 mL, 10.52 mmol) in tetrahydrofuran (20 mL) was added 5-methoxy-2-(trifluoromethyl) quinazoline (2 g, 8.76 mmol) in tetrahydrofuran (20 mL) under nitrogen at room temperature. This mixture was cooled to 0° C. and bromo (but-3-enyl) magnesium (50 mL, 26.296 mmol) was added dropwise. This mixture was stirred at room temperature overnight. The reaction was quenched with saturated solution of ammonium chloride, extracted with ethyl acetate, dried and concentrated. The residue was dissolved in anhydrous tetrahydrofuran (12 mL) and 2,3-dichloro-5,6-dicyano-1,4-benzoquinone (2.74 g, 11.83 mmol) was added in one portion. The reaction mixture was stirred for 2 h, quenched with a 2M solution of sodium hydroxide, extracted with ethyl acetate, dried and concentrated. The residue was purified using flash column chromatography, affording 4-but-3-enyl-5-methoxy-2-(trifluoromethyl)quinazoline (630 mg, 57%) as a light brown solid.

¹H NMR (400 MHZ, CDCl3-d) 7.88 (t, 1H) 7.75 (d, 1H) 7.09 (d, 1H) 5.85-6.12 (m, 1H) 5.09-5.16 (m, 1H) 5.04 (br d, 1H) 4.06 (s, 3H) 3.57-3.67 (m, 2H) 2.59 (br dd, 2H)

### Step 2: Synthesis of 4-but-3-enyl-2-(trifluoromethyl)quinazolin-5-ol

To a solution of 4-but-3-enyl-5-methoxy-2-(trifluoromethyl)quinazoline (230 mg, 0.81 mmol) in dichloromethane (2.3 mL) was added boron tribromide in dichloromethane (2.4 mL, 2.44 mmol) at 0° C. dropwise. The reaction mass was stirred at room temperature for 1 hour and quenched with aqueous solution of sodium bicarbonate solution, extracted with ethyl acetate, dried and concentrated. The residue was purified by flash column chromatography, affording 4-but-3-enyl-2-(trifluoromethyl) quinazolin-5-ol (179 mg, 82%) as a black solid.

¹H NMR (400 MHZ, CDCl₃) δ ppm 7.68-7.80 (m, 2H), 7.10 (dd, 1H), 5.98 (d, 1H), 5.09 (dd, 1H), 4.99 (dd, 1H), 3.65-3.75 (m, 2H), 2.64 (br dd, 2H)

### Step 3: Synthesis of 4-but-3-enyl-5-(5-chloropyrimidin-2-yl)oxy-2 (trifluoromethyl) quinazoline (Compound 1.043)

To a solution of 4-but-3-enyl-2-(trifluoromethyl)quinazolin-5-ol (288 mg, 1.074 mmol), in isopropyl alcohol (3 mL) was added 5-chloro-2-methylsulfonyl-pyrimidine (0.310 g, 1.61 mmol) and potassium carbonate (0.44 g, 3.22 mmol). The reaction mixture was heated at 50° C. for 16 h, then quenched with aqueous solution of ammonium chloride, extracted with ethyl acetate, dried and concentrated. The residue was purified by flash column chromatography, affording 4-but-3-enyl-5-(5-chloropyrimidin-2-yl)oxy-2 (trifluoromethyl)quinazoline (150 mg, 37%) as a yellow solid.

¹H NMR (400 MHZ, CDCl₃) δ ppm 8.53 (s, 2H), 8.14 (d, 1H), 7.99 (t, 1H), 7.48 (d, 1H), 5.86 (dd, 1H), 4.90-5.02 (m, 2H), 3.44-3.50 (m, 2H), 2.57 (td, 2H)

### Step 4: Synthesis of 4-[5-(5-chloropyrimidin-2-yl)oxy-2-(trifluoromethyl)quinazolin-4-yl]butane-1,2-diol (Compound 1.037)

To a solution of 4-but-3-enyl-5-(5-chloropyrimidin-2-yl)oxy-2-(trifluoromethyl)quinazoline (3.7 g, 9.7 mmol) in tetrahydrofuran (74 mL) and water (37 mL) was added potassium osmate (VI) dihydrate (0.28 g, 0.78 mmol) followed by 4-methyl-4-oxido-morpholin-4-ium (2.3 g, 19 mmol) at room temperature. The reaction was stirred at rt for 16 h then quenched with 10% sodium meta bisulphate solution and extracted with ethyl acetate. The combined organic layer washed with brine solution, dried and concentrated. The residue was purified by flash column chromatography, affording 4-[5-(5-chloropyrimidin-2-yl)oxy-2-(trifluoromethyl)quinazolin-4-yl]butane-1,2-diol (3.1 g, 77%) as a light brown solid.

¹H NMR (400 MHZ, CDCl₃) δ ppm 8.56 (s, 2H) 8.16 (dd, 1H) 8.03 (t, 1H) 7.58 (dd, 1H) 3.66 (d, 1H) 3.45-3.62 (m, 4H) 2.02-2.09 (m, 2H)

### Step 5: Synthesis of 3-[5-(5-chloropyrimidin-2-yl)oxy-2-(trifluoromethyl)quinazolin-4-yl]propanal (Compound 1.038)

To a solution of 4-[5-(5-chloropyrimidin-2-yl)oxy-2-(trifluoromethyl)quinazolin-4-yl]butane-1,2-diol (3.8 g, 9.2 mmol) in tetrahydrofuran (76 mL) and water (38 mL) was added sodium periodate (3.9 g, 18 mmol) at 0° C. The reaction was slowly allowed to reach room temperature then stirred for 2 h. The reaction was cooled to 5° C., quenched with 10% sodium thiosulphate solution and extracted with ethyl acetate. The combined organic layer washed with brine solution, dried and concentrated. The residue was purified by flash column chromatography, affording 3-[5-(5-chloropyrimidin-2-yl)oxy-2-(trifluoromethyl)quinazolin-4-yl]propanal (1.8 g, 4.7 mmol, 0.51, 51%) as a violet solid.

¹H NMR (400 MHZ, CDCl₃) δ ppm 9.97 (s, 1H), 8.55 (s, 2H), 8.15 (dd, 1H), 8.02 (t, 1H), 7.52 (dd, 1H), 3.77 (t, 2H), 3.08 (t, 2H)

### Step 6: Synthesis of (E)-3-[5-(5-chloropyrimidin-2-yl)oxy-2-(trifluoromethyl)quinazolin-4-yl]-N-methoxy-propan-1-imine and (Z)-3-[5-(5-chloropyrimidin-2-yl)oxy-2-(trifluoromethyl)quinazolin-4-yl]-N-methoxy-propan-1-imine

To a solution of 3-[5-(5-chloropyrimidin-2-yl)oxy-2-(trifluoromethyl)quinazolin-4-yl]propanal (0.5 g, 1.31 mmol) in acetonitrile (10 mL) was added methoxylamine hydrochloride (0.175 g, 2.09 mmol) followed by triethylamine (0.46 mL, 3.26 mmol). The reaction mixture was stirred at room temperature for 16 h then quenched with water and extracted with ethyl acetate. The combined organic layer was washed with brine solution, dried and concentrated. The residue was purified by flash column chromatography, affording (E)-3-[5-(5-chloropyrimidin-2-yl)oxy-2-(trifluoromethyl)quinazolin-4-yl]-N-methoxy-propan-1-imine (0.200 g, 37% Yield) as a white solid and (Z)-3-[5-(5-chloropyrimidin-2-yl)oxy-2-(trifluoromethyl)quinazolin-4-yl]-N-methoxy-propan-1-imine (0.108 g, 20%) as a white solid.

¹H NMR (400 MHZ, CDCl₃) δ ppm 8.54 (s, 2H), 8.15 (d, 1H), 8.01 (t, 1H), 7.47-7.54 (m, 2H), 3.74 (s, 3H), 3.62 (t, 2H), 2.77 (td, 2H) and

¹H NMR (400 MHZ, CDCl₃) δ ppm 8.54 (s, 2H), 8.15 (dd, 1H), 8.01 (t, 1H), 7.50 (dd, 1H), 6.81 (t, 1H), 3.77 (s, 3H), 3.56-3.64 (m, 2H), 2.80-2.87 (m, 2H)

### Example 2: [5-(5-chloropyrimidin-2-yl)oxy-2-(trifluoromethyl)quinazolin-4-yl]-(2-pyridyl) methanone (Compound 1.029)

### Step1: Synthesis of 5-methoxy-2-(trifluoromethyl)quinazolin-4-yl]-(2-pyridyl) methanone

To a solution of 4-bromo-5-methoxy-2-(trifluoromethyl)quinazoline (0.3 g, 0.97 mmol) in 1,4-dioxane (7 mL) was added pyridine-2-carbaldehyde (0.15 g, 1.46 mmol) and 1,3-dimethylimidazol-1-ium; iodide (0.071 g, 0.32 mmol). The reaction mixture was cooled to 0° C. and sodium hydride (0.093 g, 1.17 mmol) was added. The reaction mixture was heated to 110° C. for 1 h then cooled to room temperature, extracted with ethyl acetate, washed with brine, dried and concentrated. The residue was purified by flash column chromatography to give 5-methoxy-2-(trifluoromethyl)quinazolin-4-yl]-(2-pyridyl) methanone as an off white solid (0.120 g, 22%).

¹H NMR (400 MHZ, CDCl₃) δ ppm=8.90 (s, 1H) 8.85 (d, 1H) 8.18 (dt, 1H) 8.05 (t, 1H) 7.91 (d, 1H) 7.49 (dd, 1H) 7.10 (d, 1H) 3.75 (s, 3H)

### Step 2: Synthesis of [5-hydroxy-2-(trifluoromethyl)quinazolin-4-yl]-(2-pyridyl) methanone

To a mixture of [5-methoxy-2-(trifluoromethyl)quinazolin-4-yl]-(2-pyridyl) methanone (0.16 g, 0.48 mmol) and pyridine hydrochloride (1.43 g, 12 mmol) was heated to 190° C. for 2 h then diluted with water and extracted with ethyl acetate, dried and concentrated. The residue was purified by flash column chromatography, affording [5-hydroxy-2-(trifluoromethyl)quinazolin-4-yl]-(2-pyridyl) methanone as a white solid (0.072 g, 45%)

¹H NMR (400 MHZ, MeOD) δ ppm=8.47 (d, 1H) 8.33 (dt, 1H) 8.09 (td, 1H) 7.98 (t, 1H) 7.72 (d, 1H) 7.60 (ddd, 1H) 7.06 (dd, 1H)

### Step 3: Synthesis of [5-(5-chloropyrimidin-2-yl)oxy-2-(trifluoromethyl)quinazolin-4-yl]-(2-pyridyl) methanone (1.029)

To a solution of 2,5-dichloropyrimidine (0.017 g, 0.11 mmol) in MeCN (1.6 mL) was added (5-fluoro-2-pyridyl)-[5-hydroxy-2-(trifluoromethyl)quinazolin-4-yl]methanone (0.031 g, 0.097 mmol) and tribasic potassium phosphate (0.042 g, 0.19 mmol). The reaction mixture was heated with microwave irradiation at 130° C. for 3 h then cooled to room temperature, extracted with ethyl acetate washed with brine, dried and concentrated. The residue was purified by flash column chromatography to give [5-(5-chloropyrimidin-2-yl)oxy-2-(trifluoromethyl)quinazolin-4-yl]-(2-pyridyl) methanone (14.9 mg, 33%)

¹H NMR (400 MHZ, CDCl₃) δ ppm=8.36-8.38 (m, 1H), 8.33 (s, 2H), 8.23-8.27 (m, 2H) 8.12 (t, 1H) 7.92 (td, 1H) 7.55 (d, 1H) 7.43 (ddd, 1H)

### Example 3:1-[5-(5-chloropyrimidin-2-yl)oxy-2-(trifluoromethyl)quinazolin-4-yl]-N-(2,2,2-trifluoroethoxy) ethanimine (Compound 1.018)

### Step 1: Synthesis of 5-methoxy-2-(trifluoromethyl)-3H-quinazolin-4-one

Trifluoroacetic anhydride (66.82 mL, 463.62 mmol) was added at 0° C. to a mixture of amino-6-methoxy-benzoic acid (25 g, 149.55 mmol) and DMAP (1.84 g, 14.95 mmol) in toluene (450 mL). Triethylamine (92.4 g, 658.04 mmol) was added over a period of 45 min. the reaction was stirred at room temperature for 16 h. Ammonium acetate (52.29 g, 658.04 mmol) was added and the reaction was heated at 110° C. for 20 h. upon cooling to room temperature, the reaction was quenched with ice cold water (500 ml), stirred vigorously for 20 min, then filtered. The collected solid was washed with water, then with TMBE and dried under vacuum to afford 5-methoxy-2-(trifluoromethyl)-3H-quinazolin-4-one (30.4 g, 82%) as a cream solid.

¹H NMR (400 MHZ, DMSO-d6) δ ppm 7.80 (t, 1H), 7.32 (d, 1H), 7.20 (d, 1H), 3.90 (s, 3H)

### Step 2: Synthesis of 4-bromo-5-methoxy-2-(trifluoromethyl)quinazoline

To a suspension of 5-methoxy-2-(trifluoromethyl)quinazolin-4-ol (8.0 g, 33 mmol) in toluene (30 mL) was added phosphorus oxybromide (14 g, 49 mmol). The reaction mixture was heated at 140° C. for 3 h then cooled to room temperature and slowly poured into to saturated sodium bicarbonate solution upon which a solid precipitated. After stirring for 15 min the reaction mixture was extracted with ethyl acetate, washed with brine, dried and concentrated. The residue was purified by flash column chromatography, affording 4-bromo-5-methoxy-2-(trifluoromethyl)quinazoline (5.5 g, 55%) as an off white solid.

¹H NMR (400 MHz, CDCl₃) δ ppm 7.95 (t, 1H), 7.76 (d, 1H), 7.16 (d, 1H), 4.06 (s, 3H)

### Step 3: Synthesis of 1-[5-methoxy-2-(trifluoromethyl)quinazolin-4-yl]ethanone

To a solution of 4-bromo-5-methoxy-2-(trifluoromethyl)quinazoline (2.0 g, 6.5 mmol) in toluene (40 mL) was added tributyl(1-ethoxyvinyl)tin (3.6 g, 9.8 mmol) and tetrakis(triphenylphosphine)palladium(0) (0.23 g, 0.20 mmol). The reaction mixture was heated to 100° C. for 1 h then cooled to room temperature, quenched with saturated aqueous potassium fluoride, extracted with ethyl acetate, washed with brine, dried and concentrated. The residue was dissolved in acetonitrile (15 mL) and 2 N HCl (25 mL) then heated at 50° C. for 3 h. Upon cooling to room temperature, the reaction was extracted with ethyl acetate, washed with brine, dried and concentrated.

The residue was purified by flash column chromatography to give 1-[5-methoxy-2-(trifluoromethyl)quinazolin-4-yl]ethanone (1.2 g, 88%) as an off white solid.

¹H NMR (400 MHZ, CDCl₃) δ ppm 7.98 (t, 1H), 7.80 (d, 1H), 7.11 (d, 1H), 4.01 (s, 3H), 2.67 (s, 3H).

### Step 4: Synthesis of 1-[5-hydroxy-2-(trifluoromethyl)quinazolin-4-yl]ethanone

To a solution of 1-[5-methoxy-2-(trifluoromethyl)quinazolin-4-yl]ethanone (0.4 g, 1.48 mmol) in N-methyl-2-pyrrolidone (6 mL), was added lithium chloride (0.634 g, 14.80 mmol) and p-toluenesulfonic acid (2.57 g, 14.8 mmol). The reaction mixture was stirred at 160° C. for 5 h then cooled to room temperature, acidified with 1 N HCl and extracted with ethyl acetate. The organics were combined, washed with brine solution, dried and concentrated. The residue was purified by flash column chromatography to give 1-[5-hydroxy-2-(trifluoromethyl)quinazolin-4-yl]ethanone (0.22 g, 56%) as a gum.

¹H NMR (400 MHZ, CDCl₃) δ ppm 7.84 (t, 1H), 7.66 (d, 1H), 7.18-7.23 (m, 1H), 2.72 (s, 3H)

### Step 5: Synthesis of 4-[C-methyl-N-(2,2,2-trifluoroethoxy) carbonimidoyl]-2-(trifluoromethyl)quinazolin-5-ol

To a solution of 1-[5-hydroxy-2-(trifluoromethyl)quinazolin-4-yl]ethanone (0.15 g, 0.58 mmol) in acetonitrile (3 mL) was added 2,2,2-trifluoroethoxyammonium chloride (0.106 g, 0.7 mmol), followed by triethylamine (0.25 mL, 1.76 mmol). The reaction was stirred at 70° C. for 4 h then diluted with water, extracted with ethyl acetate, washed with brine solution, dried and concentrated. The residue was purified by flash column chromatography to give 4-[C-methyl-N-(2,2,2-trifluoroethoxy) carbonimidoyl]-2-(trifluoromethyl)quinazolin-5-ol (0.07 g, 33%) as a light brown solid.

LCMS (method 1): rt 1.13 min, 354 [M+H]⁺

### Step 6: Synthesis of 1-[5-(5-chloropyrimidin-2-yl)oxy-2-(trifluoromethyl)quinazolin-4-yl]-N-(2,2,2-trifluoroethoxy) ethanimine (Compound 1.018)

To a solution of 4-[C-methyl-N-(2,2,2-trifluoroethoxy) carbonimidoyl]-2-(trifluoromethyl)quinazolin-5-ol (0.052 g, 0.15 mmol) in isopropyl alcohol (1.0 mL) was added potassium carbonate (0.040 g, 0.29 mmol) and 5-chloro-2-methylsulfonyl-pyrimidine (0.056 g, 0.29 mmol). The reaction mixture was heated at 55° C. for 16 h then cooled to room temperature, diluted with water, extracted with ethyl acetate, washed with brine solution, dried and concentrated. The residue was purified by flash column chromatography to give 1-[5-(5-chloropyrimidin-2-yl)oxy-2-(trifluoromethyl)quinazolin-4-yl]-N-(2,2,2-trifluoroethoxy) ethanimine (0.025 g, 36%).

Major: ¹H NMR (400 MHZ, CDCl₃) δ ppm 8.49 (s, 2H), 8.21 (dd, 1H), 8.07-8.15 (m, 1H), 7.60 (dd, 1H), 4.20 (q, 2H), 2.40 (s, 3H). Minor: ¹H NMR (400 MHZ, CDCl₃) δ ppm 8.52 (s, 2H) 8.21 (dd, 1H), 8.07-8.15 (m, 1H), 7.60 (dd, 1H), 3.96-4.16 (m, 2H), 2.33 (s, 3H)

### Example 4:2-[5-(5-chloropyrimidin-2-yl)oxy-2-(trifluoromethyl)quinazolin-4-yl]-N-(cyclopropylmethoxy) ethanimine (Compound 1.008)

### Step 1: Synthesis of 5-methoxy-4-methyl-2-(trifluoromethyl)quinazoline

0.6M Lanthanum (III) trichloride bis(lithium chloride) complex in THF (30 ml, 15.78 mmol) was added to a mixture of 5-methoxy-2-(trifluoromethyl)quinazoline (3 g, 13.15 mmol) in tetrahydrofuran (60 mL), under nitrogen. The reaction mixture was cooled to 0° C. and a 3.0 M THF solution of methylmagnesiumbromide (13.15 ml, 39.44 mmol) was added. The reaction mixture was stirred at room temperature for 1 h then cooled to 0° C. and slowly quenched with saturated ammonium chloride solution, extracted with ethyl acetate, dried and concentrated. The residue was dissolved in tetrahydrofuran (40 mL) and 2,3-dichloro-5,6-dicyano-1,4-benzoquinone (7.59 g, 32.76 mmol) was added in one portion at 0° C. The reaction mixture was stirred at room temperature for 2 h then diluted with aqueous saturated sodium bicarbonate solution, extracted with ethyl acetate, washed with brine, dried and concentrated. The residue was purified by flash column chromatography to give 5-methoxy-4-methyl-2-(trifluoromethyl)quinazoline (2.5 g, 63%) as an off white solid.

¹H NMR (400 MHZ, CDCl₃) δ ppm 7.85 (t, 1H), 7.71 (d, 1H), 7.06 (d, 1H), 4.04 (s, 3H), 3.16 (s, 3H).

### Step 2: Synthesis of 4-methyl-2-(trifluoromethyl)quinazolin-5-ol

To a solution of 1-dodecanethiol (1.01 mL, 4.12 mmol) in N, N-Dimethylformamide (5.9 mL) was added lithium tert-butoxide (0.34 g, 4.13 mmol). The reaction mixture was stirred at for 15 min before 5-methoxy-4-methyl-2-(trifluoromethyl)quinazoline (0.5 g, 2.06 mmol) was added. The reaction mixture was heated at 100° C. for 4 h then cooled to room temperature, quenched with water, acidified with the 2M HCl and extracted with ethyl acetate. The organics were combined, washed with brine, dried and concentrated. The residue was purified by flash column chromatography to give 4-methyl-2-(trifluoromethyl)quinazolin-5-ol (0.47 g, 99%) as a light yellow solid.

¹H NMR (400 MHZ, CD₃OD) δ ppm 7.81 (t, 1H), 7.53 (dd, 1H), 7.11 (dd, 1H), 3.16 (s, 3H)

### Step 3: Synthesis of 5-(5-chloropyrimidin-2-yl)oxy-4-methyl-2-(trifluoromethyl)quinazoline

To a solution of 4-methyl-2-(trifluoromethyl)quinazolin-5-ol (0.75 g, 3.29 mmol) in isopropanol (15 ml) was added potassium carbonate (0.91 g, 6.57 mmol) and 5-chloro-2-methylsulfonyl-pyrimidine (1.27 g, 6.57 mmol). The reaction mixture was heated to 50° C. for 16 h then cooled to room temperature, diluted with water and extracted with ethyl acetate. The organics were combined, washed with brine, dried and concentrated. The residue was purified by flash column chromatography to give 5-(5-chloropyrimidin-2-yl)oxy-4-methyl-2-(trifluoromethyl)quinazoline (1.0 g, 89%) as an off white solid.

¹H NMR (400 MHZ, CDCl₃) δ ppm 8.52 (s, 2H), 8.13 (d, 1H), 8.00 (t, 1H), 7.49 (d, 1H), 3.01 (s, 3H)

### Step 4: Synthesis of 2-[5-(5-chloropyrimidin-2-yl)oxy-2-(trifluoromethyl)quinazolin-4-yl]-N,N-dimethyl-ethenamine (Compound 1.010)

To a solution of 5-(5-chloropyrimidin-2-yl)oxy-4-methyl-2-(trifluoromethyl)quinazoline (0.5 g, 1.47 mmol) in toluene (5 mL) was added N,N-dimethylformamide dimethyl acetal (0.82 mL, 5.87 mmol). The reaction mixture was heated with microwave irradiation at 100° C. for 2 h then cooled to room temperature and concentrated. The residue was purified by flash column chromatography to give 2-[5-(5-chloropyrimidin-2-yl)oxy-2-(trifluoromethyl)quinazolin-4-yl]-N,N-dimethyl-ethenamine (0.44 g, 76%) as a yellow solid.

¹H NMR (400 MHZ, CDCl₃) δ ppm 8.44 (s, 2H), 8.32 (d, 2H), 7.85 (dd, 1H), 7.76 (t, 1H), 7.23-7.26 (m, 1H), 6.27 (d, 1H), 3.16 (br s, 3H), 2.91 (br s, 3H)

### Step 5: Synthesis of 2-[5-(5-chloropyrimidin-2-yl)oxy-2-(trifluoromethyl)quinazolin-4-yl]-N-(cyclopropylmethoxy) ethanimine (Compound 1.008)

To a solution of 2-[5-(5-chloropyrimidin-2-yl)oxy-2-(trifluoromethyl)quinazolin-4-yl]-N,N-dimethyl-ethenamine (0.085 g, 0.215 mmol) in tetrahydrofuran (1.1 mL) was added cyclopropylmethoxyammonium chloride (0.044 g, 0.32 mmol). The reaction mixture was stirred at room temperature for 16 h then diluted with water, extracted with ethyl acetate, washed with brine, dried and concentrated. The residue was purified by flash column chromatography to give 2-[5-(5-chloropyrimidin-2-yl)oxy-2-(trifluoromethyl)quinazolin-4-yl]-N-(cyclopropylmethoxy) ethanimine (0.075 g, 64%) as a light yellow solid.

¹H NMR (400 MHZ, CDCl₃) δ ppm: Mixture of 1:1 isomers δ ppm 8.52 (s, 2H), 8.11-8.20 (m, 1H), 8.02 (td, 1H), 7.76 (t, 1H), 7.52 (t, 1H), 4.44 (d, 2H), 3.80 (d, 2H), 0.92-1.08 (m, 1H), 0.41-0.54 (m, 2H), 0.13-0.20 (m, 2H) and 8.52 (s, 2H), 8.11-8.20 (m, 1H), 8.02 (td, 1H), 7.51 (t, 1H), 7.17 (t, 1H), 4.36 (d, 2H), 3.65 (d, 2H), 0.92-1.08 (m, 1H), 0.41-0.54 (m, 2H), 0.13-0.20 (m, 2H)

### Example 5:3-[5-(5-chloropyrimidin-2-yl)oxy-2-(trifluoromethyl)quinazolin-4-yl]propan-1-ol (Compound 1.039)

To a solution of 3-[5-(5-chloropyrimidin-2-yl)oxy-2-(trifluoromethyl)quinazolin-4-yl]propanal (0.05 g, 0.13 mmol) in tetrahydrofuran (1 mL) was added portion wise sodium borohydride (0.0025 g, 0.065 mmol) at −10° C. The reaction mixture was stirred for 2 h at 0° C. then diluted with water, extracted with ethyl acetate, washed with brine, dried and concentrated. The residue was purified by flash column chromatography to give 3-[5-(5-chloropyrimidin-2-yl)oxy-2-(trifluoromethyl)quinazolin-4-yl]propan-1-ol (0.01 g, 19%) as a white solid.

### Example 6:4-(2-cyclopropylethyl)-5-(5-methylpyrimidin-2-yl)oxy-2-(trifluoromethyl)quinazoline (Compound 1.015)

### Step 1: Synthesis of 4-(2-cyclopropylethynyl)-5-methoxy-2-(trifluoromethyl) quinazoline

To a solution of 4-bromo-5-methoxy-2-(trifluoromethyl)quinazoline (2 g, 6.51 mmol) in DMF (20 mL) was added ethynyl cyclopropane (0.64 g, 9.76 mmol) followed by caesium carbonate (3.21 g, 9.76 mmol), tetrakis(triphenylphosphine)palladium(0) (0.38 g, 0.32 mmol) and cuprous iodide (0.062 g, 0.32 mmol). The reaction mixture was stirred for 16 h at room temperature then diluted with water, extracted with ethyl acetate, washed with brine, dried and concentrated. The residue was purified by flash column chromatography to give 4-(2-cyclopropylethynyl)-5-methoxy-2-(trifluoromethyl)quinazoline (1.65 g, 87%) as a light yellow solid.

¹H NMR (400 MHZ, CDCl₃) δ ppm=7.88 (t, 1H) 7.70 (d, 1H) 7.05 (d, 1H) 4.02 (s, 3H) 1.63-1.72 (m, 1H) 1.03-1.14 (m, 4H)

### Step 2: Synthesis of 4-(2-cyclopropylethyl)-5-methoxy-2-(trifluoromethyl)quinazoline

To a stirred solution of 4-(2-cyclopropylethynyl)-5-methoxy-2-(trifluoromethyl)quinazoline (0.28 g, 0.95 mmol) in methanol (20 mL) was added 5% palladium on carbon (0.28 g). The reaction mixture was stirred for 24 h under a hydrogen balloon. The reaction mixture was filtered and concentrated and the residue was purified by flash column chromatography to give 4-(2-cyclopropylethyl)-5-methoxy-2-(trifluoromethyl)quinazoline as a light yellow solid (0.22 g, 77%)

LCMS: 1.35 min, 297.2 [M+H]⁺

### Step 3: Synthesis of 4-(2-cyclopropylethyl)-2-(trifluoromethyl)quinazolin-5-ol

To a solution of 1-dodecanethiol (0.63 g, 3.0 mmol) in N, N-dimethylformamide (5 mL) was added lithium tert-butoxide (0.24 g, 3.0 mmol) followed by 4-(2-cyclopropylethyl)-5-methoxy-2-(trifluoromethyl)quinazoline (0.45 g, 1.5 mmol). The reaction mixture was heated to 70° C. for 5 h then cooled to room temperature, quenched with water, extracted with ethyl acetate, dried and concentrated. The residue was purified by flash column chromatography to give 4-(2-cyclopropylethyl)-2-(trifluoromethyl)quinazolin-5-ol (0.20 g, 47%) as a yellow solid.

¹H NMR (400 MHZ, CDCl₃) δ ppm=7.67-7.83 (m, 2H), 7.02 (dd, 1H), 6.58 (s, 1H), 3.49-3.73 (m, 2H), 1.69-1.85 (m, 2H), 0.79-0.95 (m, 1H), 0.32-0.47 (m, 2H), 0.01-0.10 (m, 2H)

### Step 4: Synthesis of 4-(2-cyclopropylethyl)-5-(5-methylpyrimidin-2-yl)oxy-2-(trifluoromethyl)quinazoline (Compound 1.015)

To a solution of 2-chloro-5-methyl-pyrimidine (0.073 g, 0.57 mmol) in isopropyl alcohol (1.6 mL) was added 4-(2-cyclopropylethyl)-2-(trifluoromethyl)quinazolin-5-ol (0.08 g, 0.28 mmol) and K₂CO₃ (0.11 g, 0.85 mmol). The reaction mixture was heated in under microwave irradiation at 150° C. for 2 h then cooled to room temperature, extracted with ethyl acetate, washed with brine, dried and concentrated. The residue was purified by flash column chromatography to give 4-(2-cyclopropylethyl)-5-(5-methylpyrimidin-2-yl)oxy-2-(trifluoromethyl)quinazoline (0.010 g, 9%) as a gum.

¹H NMR (400 MHZ, CDCl₃) δ ppm=8.42 (s, 2H), 8.09 (dd, 1H), 7.96 (t, 1H), 7.44 (dd, 1H), 3.44-3.51 (m, 2H), 2.31 (s, 3H), 1.64-1.76 (m, 2H) 0.74-0.78 (m, 1H), 0.28-0.39 (m, 2H), 0.02 (q, 2H)

### Example 7: Synthesis of 5-(5-chloropyrimidin-2-yl)oxy-4-[2-(1-methoxy-4-piperidyl)ethyl]-2-(trifluoromethyl)quinazoline (Compound 1.023)

### Step1: Synthesis of 5-fluoro-2-(trifluoromethyl)quinazolin-4-ol

To a solution of 2-amino-6-fluoro-benzoic acid (5 g, 32.23 mmol) and dimethyl aminopyridine (0.39 g, 3.22 mmol) in toluene (96 mL) was added dropwise trifluoroacetic anhydride (14.40 mL, 99.9 mmol) at 0° C. followed by triethylamine (22 mL, 161.16 mmol) over a period of 45 min. The reaction mixture was stirred at room temperature overnight then ammonium acetate (11.27 g, 141.82 mmol) was added and resultant reaction mixture was heated at reflux for 30 h. The reaction mixture was cooled to room temperature, diluted with water and stirred for 20 min, upon which collection of the precipitated solid afforded 5-fluoro-2-(trifluoromethyl) quinazolin-4-ol (4 g, 53%) as a brown solid.

¹H NMR (400 MHZ, CDCl₃) δ ppm=7.82 (td, 1H), 7.68 (d, 1H), 7.27-7.34 (m, 1H)

### Step 2: Synthesis of 5-benzyloxy-2-(trifluoromethyl)quinazolin-4-ol

To a solution of benzyl alcohol (3.2 g, 29 mmol) in N, N-Dimethylacetamide (110 mL) at 0° C. was added portion wise sodium hydride (2.1 g, 49 mmol) and the reaction mixture was stirred for 1 h. 5-fluoro-2-(trifluoromethyl)quinazolin-4-ol (5.7 g, 25 mmol) was added and the reaction mixture was stirred at room temperature overnight, quenched with ice cold solution of ammonium chloride, extracted with ethyl acetate, washed with brine, dried and concentrated. The residue was purified by flash column chromatography to give 5-benzyloxy-2-(trifluoromethyl)quinazolin-4-ol (3.5 g, 46%) as a white solid.

¹H NMR (400 MHZ, CDCl₃) δ ppm=7.72 (t, 1H), 7.57 (d, 2H), 7.29-7.48 (m, 4H), 7.10 (d, 1H), 5.31 (s, 2H)

### Step 3: Synthesis of 5-benzyloxy-4-chloro-2-(trifluoromethyl)quinazoline

To a solution of 5-benzyloxy-2-(trifluoromethyl)quinazolin-4-ol (1.3 g, 4.1 mmol) in thionyl chloride (26 mL) was added N, N-dimethylformamide (0.032 mL, 0.41 mmol). The reaction mixture was heated to 60° C. for 8 h then concentrated, cooled to 0° C. and quenched by the addition of saturated solution of sodium bicarbonate to neutral pH. The resultant mixture was extracted with ethyl acetate, washed with brine, dried and concentrated. The residue was purified by flash column chromatography to give 5-benzyloxy-4-chloro-2-(trifluoromethyl)quinazoline (0.950 g, 69%) as an off white solid.

¹H NMR (400 MHZ, CDCl₃) δ ppm=7.92 (t, 1H) 7.79 (d, 1H) 7.53 (br d, 2H) 7.35-7.48 (m, 3H) 7.23 (d, 1H) 5.33 (s, 2H)

### Step 4: Synthesis of 5-benzyloxy-4-[2-(1-methoxy-4-piperidyl) ethynyl]-2-(trifluoromethyl)quinazoline

To a stirred solution of 5-benzyloxy-4-chloro-2-(trifluoromethyl)quinazoline (0.5 g, 1.47 mmol) in tetrahydrofuran (15 mL) was added 4-ethynyl-1-methoxy-piperidine (0.41 g, 2.9 mmol), Copper (I) iodide (0.056 g, 0.29 mmol) and dichlorobis (triphenylphosphine) palladium(II) (0.21 g, 0.29 mmol) followed by triethylamine (0.62 mL, 4.4 mmol). The reaction mixture was stirred at room temperature overnight then quenched with water, extracted with ethyl acetate, washed with brine, dried and concentrated. The residue was purified by flash column chromatography to give 5-benzyloxy-4-[2-(1-methoxy-4-piperidyl) ethynyl]-2-(trifluoromethyl)quinazoline (0.590 g, 90%) as a yellow gum.

LCMS: 1.37 min, 440.1 [M−H]⁺

### Step 5: Synthesis of 4-[2-(1-methoxy-4-piperidyl)ethyl]-2-(trifluoromethyl)quinazolin-5-ol

To a solution of 5-benzyloxy-4-[2-(1-methoxy-4-piperidyl) ethynyl]-2-(trifluoromethyl) quinazoline (0.5 g, 1.13 mmol) and triethylamine (10 mL) in methanol (50 mL) was added 5% palladium on carbon (1 g). The reaction mixture was stirred at room temperature for 3 hours under 1 bar hydrogen atmosphere. The reaction mixture was filtered and concentrated and the residue was purified by flash column chromatography to give 4-[2-(1-methoxy-4-piperidyl)ethyl]-2-(trifluoromethyl) quinazolin-5-ol (0.06 g, 15%) as a green solid.

LCMS (method 1): rt 2.86 min, 356.2 [M+H]⁺

### Step 6: Synthesis of 5-(5-chloropyrimidin-2-yl)oxy-4-[2-(1-methoxy-4-piperidyl)ethyl]-2-(trifluoromethyl)quinazoline (Compound 1.023)

To a solution of 4-[2-(1-methoxy-4-piperidyl)ethyl]-2-(trifluoromethyl)quinazolin-5-ol (0.06 g, 0.18 mmol) in isopropyl alcohol (3 mL) was added potassium carbonate (0.072 g, 0.5 mmol) followed by 5-methyl-2-methylsulfonyl-pyrimidine (0.097 g, 0.61 mmol). The reaction mixture was heated to 60° C. for 16 h then cooled to room temperature, extracted with ethyl acetate, washed with brine, dried and concentrated. The residue was purified by flash column chromatography to give 5-(5-chloropyrimidin-2-yl)oxy-4-[2-(1-methoxy-4-piperidyl)ethyl]-2-(trifluoromethyl) quinazoline (0.030 g, 38%) as an off white solid.

¹H NMR (400 MHZ, CDCl₃) δ ppm=8.53 (s, 2H), 8.13 (d, 1H), 7.98 (t, 1H), 7.46 (d, 1H), 3.52 (s, 3H), 3.31-3.39 (m, 4H), 2.23-2.29 (m, 2H), 1.64-1.72 (m, 4H), 1.20-1.30 (m, 3H)

### Example 8: Synthesis of 5-(5-chloropyrimidin-2-yl)oxy-2-(trifluoromethyl)-4-[2-(trifluoromethyl)pyrimidin-5-yl]quinazoline (Compound 1.013)

### Step1: Synthesis of 4-bromo-5-methoxy-2-(trifluoromethyl)quinazoline

To a solution of 5-methoxy-2-(trifluoromethyl)quinazolin-4-ol (3.2 g, 13 mmol) in toluene (15 mL) was added phosphorus oxybromide (5.7 g, 20 mmol) and the reaction mixture was heated to 140° C. for 3 h. Upon cooling, the solution was added dropwise to saturated sodium bicarbonate solution then extracted with ethyl acetate, washed with brine, dried and concentrated. The residue was purified by flash column chromatography to give 4-bromo-5-methoxy-2-(trifluoromethyl)quinazoline (1.6 g, 40%) as a white solid.

¹H NMR (400 MHZ, CDCl₃) δ ppm=7.96 (t, 1H), 7.78 (d, 1H), 7.18 (d, 1H) 4.07 (s, 3H)

### Step 2: Synthesis of 5-methoxy-2-(trifluoromethyl)-4-[2-(trifluoromethyl)pyrimidin-5-yl]quinazoline

To a solution of 4-bromo-5-methoxy-2-(trifluoromethyl)quinazoline (0.25 g, 0.81 mmol) in dry 2-methyltetrahydrofuran (15 mL) was added (2-(trifluoromethyl)pyrimidin-5-yl) boronic acid (0.234 g, 1.2 mmol) and cesium carbonate (0.66 g, 2.0 mmol). The reaction mixture was degassed with nitrogen then [1,1′bis(diphenylphosphino)ferrocene] dichloropalladium(II) (0.07 g, 0.09 mmol) was added and the reaction mixture was heated at 90° C. for 2 h. Upon cooling to room temperature, the reaction mixture was quenched with water, extracted with ethyl acetate, dried and concentrated. The residue was purified by flash column chromatography to give 5-methoxy-2-(trifluoromethyl)-4-[2-(trifluoromethyl)pyrimidin-5-yl]quinazoline (0.28 g, 92%) as an off white solid.

¹H NMR (400 MHZ, CDCl₃) δ ppm=9.10 (s, 2H), 8.06 (t, 1H), 7.94 (d, 1H), 7.17 (d, 1H), 3.79 (s, 3H)

### Step 3: Synthesis of 2-(trifluoromethyl)-4-[2-(trifluoromethyl)pyrimidin-5-yl]quinazolin-5-ol

To a solution of 1-dodecanethiol (0.28 g, 1.39 mmol) in N, N-dimethylformamide (2.6 mL) was added lithium tert-butoxide (0.11 g, 1.4 mmol) followed by 5-methoxy-2-(trifluoromethyl)-4-[2-(trifluoromethyl)pyrimidin-5-yl]quinazoline (0.26 g, 0.69 mmol). The reaction mixture was heated to 100° C. for 2 h then cooled to room temperature, quenched with water, acidified using 2N HCl, extracted with ethyl acetate, dried and concentrated. The residue was purified by flash column chromatography to give 2-(trifluoromethyl)-4-[2-(trifluoromethyl)pyrimidin-5-yl]quinazolin-5-ol (0.17 g, 49%) as a yellow solid.

¹H NMR (400 MHZ, CD₃CN) δ ppm=9.17 (s, 2H), 8.58 (br s, 1H), 8.01 (t, 1H), 7.79 (d, 1H), 7.24-7.29 (m, 1H)

### Step 4: Synthesis of 5-(5-chloropyrimidin-2-yl)oxy-2-(trifluoromethyl)-4-[2-(trifluoromethyl)pyrimidin-5-yl]quinazoline (Compound 1.013)

To a solution of 2-(trifluoromethyl)-4-[2-(trifluoromethyl)pyrimidin-5-yl]quinazolin-5-ol (0.16 g, 0.44 mmol) in isopropyl alcohol (3.2 mL) was added potassium carbonate (0.12 g, 0.89 mmol) followed by 5-methyl-2-methylsulfonyl-pyrimidine (0.17 g, 0.89 mmol). The reaction mixture was heated to 55° C. for 16 h then cooled to room temperature, extracted with ethyl acetate, washed with brine, dried and concentrated. The residue was purified by flash column chromatography to give 5-(5-chloropyrimidin-2-yl)oxy-2-(trifluoromethyl)-4-[2-(trifluoromethyl)pyrimidin-5-yl]quinazoline (0.082 g, 39%) as a light yellow solid.

¹H NMR (400 MHZ, CDCl₃) δ ppm=8.93 (s, 2H), 8.34 (dd, 1H), 8.23 (s, 2H), 8.20 (t, 1H), 7.64 (dd, 1H)

### Example 9: Synthesis of 1-[5-(5-chloropyrimidin-2-yl)oxy-2-(trifluoromethyl)quinazolin-4-yl]ethenone (Compound 1.028)

### Step 1: Synthesis of 4-ethyl-5-methoxy-2-(trifluoromethyl)quinazoline

To a solution of 5-methoxy-2-(trifluoromethyl)quinazoline (0.4 g, 1.7 mmol) in tetrahydrofuran (4.8 mL) was added bromo (ethyl) magnesium (2.65 g, 5.26 mmol) at 0° C. over a period of 20 min. The reaction mixture was stirred at room temperature for 12 h then quenched with ammonium chloride, extracted with ethyl acetate, dried and concentrated. The residue was diluted with tetrahydrofuran (5.1 mL) and 2,3-dichloro-5,6-dicyano-1,4-benzoquinone (0.91 g, 3.95 mmol) was added at 0° C. The reaction mixture was stirred at room temperature for 2 h then cooled to 10° C., quenched with saturated sodium bicarbonate solution, extracted with ethyl acetate, washed with brine solution, dried and concentrated. The residue was purified by flash column chromatography to give 4-ethyl-5-methoxy-2-(trifluoromethyl)quinazoline (0.325 g, 64% Yield) as a white solid.

¹H NMR (400 MHZ, CDCl₃) δ ppm 7.86 (t, 1H), 7.75 (d, 1H), 7.08 (d, 1H), 4.07 (s, 3H), 3.55 (d, 2H), 1.40 (t, 3H)

### Step 2: Synthesis of 4-ethyl-2-(trifluoromethyl)quinazolin-5-ol

To a solution of 1-dodecanethiol (0.51 g, 2.5 mmol) in N, N-dimethylformamide (3.57 mL) was added lithium tert-butoxide (0.2 g, 2.5 mmol). The reaction mixture was stirred for 15 min and 4-ethyl-5-methoxy-2-(trifluoromethyl)quinazoline (0.32 g, 1.25 mmol) was added. The reaction mixture was heated at 100° C. for 2.5 h then cooled to room temperature, quenched with ice cold water, acidified with 2 N HCl and extracted with ethyl acetate. The combined organics were washed with brine solution, dried and concentrated. The residue was purified by flash column chromatography to give 4-ethyl-2-(trifluoromethyl)quinazolin-5-ol (0.19 g, 43% Yield) as an off white solid.

¹H NMR (400 MHZ, CDCl₃) δ ppm 7.75 (m, 1H), 7.06 (dd, 1H), 7.00 (s, 1H), 3.62 (q, 2H), 1.44 (t, 3H)

### Step 3: Synthesis of 5-(5-chloropyrimidin-2-yl)oxy-4-ethyl-2-(trifluoromethyl) quinazoline

To a solution of 4-ethyl-2-(trifluoromethyl)quinazolin-5-ol (0.17 g, 0.7 mmol) in isopropyl alcohol (2.55 mL) were added potassium carbonate (0.29 g, 2.1 mmol) and 5-chloro-2-methylsulfonyl-pyrimidine (0.2 g, 1.05 mmol). The reaction mixture was heated at 50° C. for 16 h then cooled to room temperature, quenched with water, extracted with ethyl acetate, dried and concentrated. The residue was purified by flash column chromatography to give 5-(5-chloropyrimidin-2-yl)oxy-4-ethyl-2-(trifluoromethyl)quinazoline (0.17 g, 70%) as a white solid.

¹H NMR (400 MHZ, CDCl₃) δ ppm 8.53 (s, 2H), 8.14 (d, 1H), 7.99 (t, 1H), 7.47 (d, 1H), 3.39 (q, 2H), 1.34 (t, 3H)

### Step 4: Synthesis of 4-(1-bromoethyl)-5-(5-chloropyrimidin-2-yl)oxy-2-(trifluoromethyl) quinazoline (1.001)

To a solution of 5-(5-chloropyrimidin-2-yl)oxy-4-ethyl-2-(trifluoromethyl)quinazoline (0.1 g, 0.28 mmol) in carbon tetrachloride (2.25 mL) were added N-bromosuccinimide (0.05 g, 0.31 mmol) and azobisisobutyronitrile (0.015 g, 0.085 mmol). The reaction mixture was heated at 85° C. for 2 h then cooled to room temperature, quenched with water and extracted with ethyl acetate. The combined organics were dried and concentrated and the residue was purified by flash column chromatography to give 4-(1-bromoethyl)-5-(5-chloropyrimidin-2-yl)oxy-2-(trifluoromethyl)quinazoline (0.118 g, 87%) as a white solid.

¹H NMR (400 MHZ, CDCl₃) δ ppm 8.55 (s, 2H) 8.19 (dd, 1H) 8.05 (t, 1H) 7.61 (dd, 1H) 7.6.49 (d, 1H) 2.19 (m, 3H)

### Step 5: Synthesis of 1-[5-(5-chloropyrimidin-2-yl)oxy-2-(trifluoromethyl)quinazolin-4-yl]ethenone (1.028)

To a solution of 4-(1-bromoethyl)-5-(5-chloropyrimidin-2-yl)oxy-2-(trifluoromethyl)quinazoline (0.095 g, 0.22 mmol) in acetonitrile (2.19 mL) was added dimethylsulfoxide (0.03 mL) and 4-methyl-4-oxido-morpholin-4-ium (0.1 g, 0.09 mL, 0.43 mmol). The reaction mixture was stirred at room temperature for 16 h then quenched with water and extracted with ethyl acetate, dried and concentrated. The residue was purified by flash column chromatography to give 1-[5-(5-chloropyrimidin-2-yl)oxy-2-(trifluoromethyl)quinazolin-4-yl]ethanone (0.034 g, 40%) as a gum.

¹H NMR (400 MHZ, CDCl₃) δ ppm: 8.52 (s, 2H) 8.19-8.23 (m, 1H) 8.11-8.18 (m, 1H) 7.63-7.68 (m, 1H) 2.78 (s, 3H)

### Example 10:5-[5-(5-chloropyrimidin-2-yl)oxy-2-(trifluoromethyl)quinazolin-4-yl]-3-(difluoromethyl)isoxazole (Compound 1.035)

### Step-1: Synthesis of 1-[5-methoxy-2-(trifluoromethyl)quinazolin-4-yl]ethenone

To a solution of 1-[5-methoxy-2-(trifluoromethyl)quinazolin-4-yl]ethanone (1.8 g, 6.7 mmol) in tetrahydrofuran (14 mL) was added portionwise 60% sodium hydride in mineral oil (0.64 g, 13 mmol) at 0° C. After stirring for 10 minutes, a solution of ethyl 2,2-difluoroacetate (1.7 g, 13 mmol) in tetrahydrofuran (3.6 mL) was added dropwise. The reaction mixture was stirred at room temperature for 16 h then quenched with ice cold water, acidified to pH 3 using 2M HCl and extracted with ethyl acetate. The combined organic layer was washed with brine solution, dried and concentrated. The residue was purified by flash column chromatography to give 4,4-difluoro-1-[5-methoxy-2-(trifluoromethyl)quinazolin-4-yl]butane-1,3-dione (1.6 g, 58%) as a gum.

¹H NMR (400 MHZ, CDCl₃) δ ppm 7.96-8.09 (m, 1H), 7.85 (d, 1H), 7.15 (d, 1H), 6.22 (s, 2H), 5.96 (t, 1H), 3.97 (s, 3H)

### Step-2: Synthesis of afforded 3-(difluoromethyl)-5-[5-methoxy-2-(trifluoromethyl)quinazolin-4-yl]isoxazole

To a solution of 4,4-difluoro-1-[5-methoxy-2-(trifluoromethyl)quinazolin-4-yl]butane-1,3-dione (0.8 g, 2.30 mmol) in ethanol (8 mL) was added hydroxylamine hydrochloride (0.25 g, 3.45 mmol). The reaction mixture was heated at 80° C. for 5 h then cooled to room temperature, diluted with water and extracted with ethyl acetate. The organics were combined, washed with brine, dried and concentrated. The residue was purified by flash column chromatography to give 3-(difluoromethyl)-5-[5-methoxy-2-(trifluoromethyl)quinazolin-4-yl]isoxazole (0.56 g, 70%) as a gum.

¹H NMR (400 MHZ, CDCl₃) δ ppm 8.04 (t, 1H), 7.95 (dd, 1H), 7.30 (d, 1H), 7.24 (s, 1H), 6.62 (t, 1H), 4.10 (s, 3H)

### Step-3: Synthesis of 4-[3-(difluoromethyl)isoxazol-5-yl]-2-(trifluoromethyl)quinazolin-5-ol

To 3-(difluoromethyl)-5-[5-methoxy-2-(trifluoromethyl)quinazolin-4-yl]isoxazole (0.34 g, 0.98 mmol) was added pyridine hydrochloride (3.48 g, 29.55 mmol) and the reaction mixture was heated at 180° C. for 2 h. The mixture was brought to room temperature before being diluted with water, acidified with 2 N HCl and extracted with ethyl acetate. The combined organics were dried and concentrated and the residue was purified by flash column chromatography to give 4-[3-(difluoromethyl)isoxazol-5-yl]-2-(trifluoromethyl)quinazolin-5-ol (0.13 g, 40%) as a yellow solid.

¹H NMR (400 MHZ, CD₃OD) δ ppm 7.99-8.05 (m, 1H), 7.73-7.77 (m, 1H), 7.18-7.23 (m, 2H), 7.09 (t, 1H)

### Step 5: Synthesis of 5-[5-(5-chloropyrimidin-2-yl)oxy-2-(trifluoromethyl)quinazolin-4-yl]-3-(difluoromethyl)isoxazole (Compound 1.035)

To a solution of 4-[3-(difluoromethyl)isoxazol-5-yl]-2-(trifluoromethyl)quinazolin-5-ol (0.11 g, 0.33 mmol) in isopropyl alcohol (2.2 mL) was added potassium carbonate (0.09 g, 0.66 mmol) and 5-chloro-2-methylsulfonyl-pyrimidine (0.13 g, 0.66 mmol). The reaction mixture was heated at 55° C. for 16 h then cooled to room temperature, diluted with water and extracted with ethyl acetate. The combined organics were washed with brine solution, dried and concentrated. The residue was purified by flash column chromatography to give 5-[5-(5-chloropyrimidin-2-yl)oxy-2-(trifluoromethyl)quinazolin-4-yl]-3-(difluoromethyl)isoxazole (0.022 g, 15%) as a light yellow solid.

¹H NMR (400 MHZ, CDCl₃) δ ppm 8.39 (s, 2H), 8.30 (d, 1H), 8.19 (t, 1H), 7.66 (d, 1H), 6.86 (s, 1H), 6.80 (t, 1H)

### Example 11:1-[5-(5-chloropyrimidin-2-yl)oxy-2-(trifluoromethyl)quinazolin-4-yl]-N-(cyclopropylmethoxy)methanimine (Compound 1.004)

### Step-1: Synthesis of 5-methoxy-2-(trifluoromethyl)-4-vinyl-quinazoline

To a solution of 4-bromo-5-methoxy-2-(trifluoromethyl)quinazoline (2.5 g, 8.1 mmol) in toluene (50 mL) was added tributyl(vinyl)tin (3.6 mL, 12 mmol) and tetrakis(triphenylphosphine)palladium(0) (0.28 g, 0.24 mmol). The reaction mixture was heated to 100° C. for 2 h, cooled to room temperature and 1M aqueous solution of potassium fluoride was added. The resulting solution was stirred for 15 mins and then filtered over a bed of celite and washed through with ethyl acetate. The aqueous layer was extracted with ethyl acetate and the combined organics were dried and concentrated. The residue was purified by flash column chromatography to give 5-methoxy-2-(trifluoromethyl)-4-vinyl-quinazoline (1.03 g, 50%) as an off white solid.

¹H NMR (400 MHZ, CHLOROFORM-d) δ ppm 8.04-8.17 (m, 1H), 7.79-7.92 (m, 1H), 7.73 (br d, 1H), 7.00-7.15 (m, 1H), 6.84 (dd, 1H), 5.82 (dd, 1H), 4.06 (s, 3H)

### Step 2: Synthesis of 1-[5-methoxy-2-(trifluoromethyl)quinazolin-4-yl]ethane-1,2-diol

To a solution of 5-methoxy-2-(trifluoromethyl)-4-vinyl-quinazoline (1.0 g, 3.9 mmol) in tetrahydrofuran (20 mL) and water (10 mL) was added potassium osmate (VI) dihydrate (0.12 g, 0.31 mmol) and 4-methyl-4-oxido-morpholin-4-ium (0.94 g, 7.9 mmol). The reaction mixture was stirred at room temperature for 16 h then quenched with 10% sodium thiosulphate solution and extracted with 10% methanol in ethyl acetate. The combined organics were washed with brine solution, dried and concentrated to give 1-[5-methoxy-2-(trifluoromethyl)quinazolin-4-yl]ethane-1,2-diol (1.05 g, 93%) as a light brown solid.

¹H NMR (400 MHZ, DMSO-d6) δ ppm 8.08 (t, 1H), 7.75 (d, 1H), 7.43 (d, 1H), 5.86-5.92 (m, 1H), 5.13 (d, 1H), 4.70 (t, 1H), 4.06 (s, 3H), 3.82-3.90 (m, 1H), 3.63 (dt, 1H)

### Step 3: Synthesis of 5-methoxy-2-(trifluoromethyl)quinazoline-4-carbaldehyde

To a solution of 1-[5-methoxy-2-(trifluoromethyl)quinazolin-4-yl]ethane-1,2-diol (0.77 g, 2.67 mmol) in tetrahydrofuran (15 mL) and water (7.7 mL) was added sodium periodate (1.15 g, 5.34 mmol). The reaction mixture was stirred at room temperature for 2 h then quenched with 10% solution sodium thiosulphate and stirred for 15 minutes. The mixture was diluted with water and extracted with ethyl acetate. The combined organics were washed with brine solution, dried and concentrated to give 5-methoxy-2-(trifluoromethyl)quinazoline-4-carbaldehyde (0.55 g, 81%) as a gum.

¹H NMR (400 MHZ, CHLOROFORM-d) δ ppm 10.74 (s, 1H) 8.04 (t, 1H) 7.88 (d, 1H) 7.19 (d, 1H) 4.11 (s, 3H)

### Step-4: Synthesis of 5-hydroxy-2-(trifluoromethyl)quinazoline-4-carbaldehyde

To a solution of 5-methoxy-2-(trifluoromethyl)quinazoline-4-carbaldehyde (0.22 g, 0.86 mmol) in 1,2-dichloroethane (5 mL) was added boron tribromide in dichloromethane (9.9 mL, 9.87 mmol) at 0° C. The reaction mixture was heated at 60° C. for 3.5 h then cooled to 0° C. and basified with 5% sodium hydroxide solution. The aqueous layer was washed with ethyl acetate then acidified with 2N hydrochloric acid solution and extracted with ethyl acetate. The combined organics were washed with water and concentrated to give 5-hydroxy-2-(trifluoromethyl)quinazoline-4-carbaldehyde (0.13 g, 62%) as a gum.

¹H NMR (400 MHZ, CD₃CN) δ ppm 10.65 (s, 1H) 9.70 (s, 1H) 8.04 (t, 1H) 7.77 (d, 1H) 7.34 (d, 1H)

### Step 5: Synthesis of 4-(cyclopropylmethoxyiminomethyl)-2-(trifluoromethyl) quinazolin-5-ol

To a solution of 5-hydroxy-2-(trifluoromethyl)quinazoline-4-carbaldehyde (0.13 g, 0.53 mmol) in acetonitrile (1.95 mL) was added N,N-diethylethanamine (0.23 mL, 1.61 mmol) and cyclopropylmethoxyammonium chloride (0.2 g, 1.61 mmol). The reaction mixture was stirred at room temperature for 6 h then diluted with water, extracted with ethyl acetate, washed with brine solution and concentrated. The residue was purified by flash column chromatography to give 4-(cyclopropylmethoxyiminomethyl)-2-(trifluoromethyl)quinazolin-5-ol (0.045 g, 26%) as an orange solid.

¹H NMR (400 MHZ, CHLOROFORM-d) δ ppm 12.15 (s, 1H) 8.56 (s, 1H) 7.93 (t, 1H) 7.77 (dd, 1H) 7.29 (d, 1H), 4.23 (d, 2H), 1.22-1.32 (m, 1H) 0.64-0.77 (m, 2H) 0.37-0.49 (m, 2H)

### Step 6: Synthesis of 1-[5-(5-chloropyrimidin-2-yl)oxy-2-(trifluoromethyl)quinazolin-4-yl]-N-(cyclopropylmethoxy)methanimine (Compound 1.004)

To a solution of 4-(cyclopropylmethoxyiminomethyl)-2-(trifluoromethyl)quinazolin-5-ol (0.04 g, 0.13 mmol) in isopropyl alcohol (1 mL) was added 5-chloro-2-(methylsulfonyl)pyrimidine (0.1 g, 0.5 mmol) and potassium carbonate (0.05 g 0.38 mmol). The reaction mixture was heated at 50° C. for 8 h then quenched with water, extracted with ethyl acetate, washed with water and brine, dried and concentrated. The residue was purified by flash column chromatography to give 1-[5-(5-chloropyrimidin-2-yl)oxy-2-(trifluoromethyl)quinazolin-4-yl]-N-(cyclopropyl methoxy)methanimine (0.007 g, 13%) as a gum.

¹H NMR (400 MHZ, CHLOROFORM-d) δ ppm 8.95 (s, 1H) 8.48 (s, 2H) 8.20 (d, 1H) 8.09 (t, 1H) 7.60 (d, 1H), 3.97 (d, 2H) 1.00-1.18 (m, 1H), 0.51-0.60 (m, 2H) 0.23-0.30 (m, 2H)

### Example 12:3-[5-(5-chloropyrimidin-2-yl)oxy-2-(trifluoromethyl)quinazolin-4-yl]-N-ethyl-propanamide (Compound 1.012)

To a solution of 3-[5-(5-chloropyrimidin-2-yl)oxy-2-(trifluoromethyl)quinazolin-4-yl]propanal (0.05 g, 0.131 mmol) in acetonitrile (2.61 mL) was added N-hydroxysuccinimide (0.017 g, 0.14 mmol) and diacetoxyiodo)benzene (0.047 g, 0.14 mmol). The reaction mixture was stirred at room temperature for 3 min before 2.0 M ethyl amine in THF (0.13 mL, 0.26 mmol) was added. The reaction mixture was stirred at room temperature for 16 h, diluted with water and 10% solution of sodium thiosulphate and extracted with ethyl acetate. The combined organics were washed with brine, dried and concentrated. The residue was purified by flash column chromatography to give 3-[5-(5-chloropyrimidin-2-yl)oxy-2-(trifluoromethyl) quinazolin-4-yl]-N-ethyl-propanamide (0.027 g, 48%) as an off white solid.

¹H NMR: (400 MHZ, DMSO-d6) δ ppm 8.83 (s, 2H), 8.12-8.22 (m, 2H), 7.84 (br s, 1H), 7.78-7.82 (m, 1H), 3.56 (t, 2H), 2.99 (dd, 2H), 2.56-2.63 (m, 2H), 0.95 (t, 3H)

### Example 13:5-(5-chloropyrimidin-2-yl)oxy-4-[5-(difluoromethyl)-1-methyl-pyrazol-3-yl]-2-(trifluoromethyl)quinazoline (Compound 1.016)

### Step-1: Synthesis of 4-[5-(difluoromethyl)-1-methyl-pyrazol-3-yl]-5-methoxy-2-(trifluoromethyl)quinazoline

To a solution of 4,4-difluoro-1-[5-methoxy-2-(trifluoromethyl)quinazolin-4-yl]butane-1,3-dione (0.9 g, 2.3 mmol) in acetonitrile (9 mL) was added pyridine (0.63 g, 0.64 mL, 7.9 mmol) and methylhydrazine sulfate (0.5 g, 3.5 mmol). The reaction mixture was stirred at room temperature overnight then quenched with ice cold water and extracted with ethyl acetate. The combined organics were washed with brine, dried and concentrated. The residue was purified by flash column chromatography to give 4-[5-(difluoromethyl)-1-methyl-pyrazol-3-yl]-5-methoxy-2-(trifluoromethyl)quinazoline (0.37 g, 44%) as a light brown solid.

¹H NMR (400 MHZ, CDCl₃) δ ppm 7.94 (t, 1H), 7.78-7.83 (m, 1H), 7.08 (d, 1H), 6.82 (t, 1H), 4.10 (s, 3H), 3.79 (s, 3H).

### Step-2: Synthesis of 4-[5-(difluoromethyl)-1-methyl-pyrazol-3-yl]-2-(trifluoromethyl)quinazolin-5-ol

A mixture of 4-[5-(difluoromethyl)-1-methyl-pyrazol-3-yl]-5-methoxy-2-(trifluoromethyl) quinazoline (0.26 g, 0.72 mmol) and pyridine hydrochloride (2.5 g, 21.77 mmol) was heated to 180° C. for 2 h. The reaction mixture was brought to room temperature, diluted with water, acidified with 2N HCl and extracted with ethyl acetate. The combined organic layer was washed with brine, dried and concentrated. The residue was purified by flash column chromatography to give 4-[5-(difluoromethyl)-1-methyl-pyrazol-3-yl]-2-(trifluoromethyl)quinazolin-5-ol (0.2 g, 80%) as a yellow solid.

¹H NMR (400 MHZ, CD₃OD) δ ppm 7.95 (t, 1H), 7.67 (dd, 1H), 7.51 (s, 1H), 7.25 (dd, 1H), 7.19 (t, 1H), 4.17 (s, 3H)

### Step-3: Synthesis of 5-(5-chloropyrimidin-2-yl)oxy-4-[5-(difluoromethyl)-1-methyl-pyrazol-3-yl]-2-(trifluoromethyl)quinazoline

To a solution of 4-[5-(difluoromethyl)-1-methyl-pyrazol-3-yl]-2-(trifluoromethyl)quinazolin-5-ol (0.2 g, 0.58 mmol) in isopropyl alcohol (4 mL) was added potassium carbonate (0.16 g, 1.16 mmol) and 5-chloro-2-methylsulfonyl-pyrimidine (0.22 g, 1.16 mmol). The reaction mixture was heated to 55° C. for 16 h then cooled to room temperature, diluted with water and extracted with ethyl acetate. The combined organic layer was washed with brine solution, dried and concentrated. The residue was purified by flash column chromatography to give 5-(5-chloropyrimidin-2-yl)oxy-4-[5-(difluoromethyl)-1-methyl-pyrazol-3-yl]-2-(trifluoromethyl) quinazoline (0.09 g, 33%) as a light yellow solid.

¹H NMR (400 MHZ, CDCl₃) δ ppm 8.29 (s, 2H), 8.23 (dd, 1H), 8.06-8.13 (m, 1H), 7.59 (dd, 1H), 6.71 (d, 1H), 6.70 (t, 2H), 3.87 (s, 3H)

### Example 14: Synthesis of 5-(5-chloropyrimidin-2-yl)oxy-4-cyclopropyl-2-(trifluoromethyl)quinazoline (Compound 1.041)

### Step 1: Synthesis of 4-cyclopropyl-5-methoxy-2-(trifluoromethyl)quinazoline

To a solution of 4-bromo-5-methoxy-2-(trifluoromethyl)quinazoline (0.5 g, 1.62 mmol) in toluene (15 mL) was added cyclopropylboronic acid (0.42 g, 4.9 mmol), tricyclohexylphosphine (0.045 g, 0.16 mmol), tribasic potassium phosphate (0.87 g, 6.5 mmol) and palladium acetate (0.018 g, 0.08 mmol). The reaction mixture was heated at 90° C. for 16 h then cooled to room temperature, quenched with water, extracted with ethyl acetate, dried and concentrated. The residue was purified by flash column chromatography to give 4-cyclopropyl-5-methoxy-2-(trifluoromethyl)quinazoline (0.15 g, 31%) as a white solid.

¹H NMR (400 MHZ, CDCl₃) δ ppm 7.83 (t, 1H), 7.70 (dd, 1H), 7.08 (d, 1H), 4.07 (s, 3H) 3.65 (m, 1H), 1.50 (dd, 2H), 1.25 (dd, 2H)

### Step 2: Synthesis of 4-cyclopropyl-2-(trifluoromethyl)quinazolin-5-ol

To a solution of 1-dodecanethiol (0.22 mL) in N, N-dimethylformamide (1.2 mL) was added lithium tert-butoxide (0.07 g, 0.89 mmol). After stirring for 10 min, 4-cyclopropyl-5-methoxy-2-(trifluoromethyl)quinazoline (0.12 g, 0.44 mmol) was added and the reaction mixture was heated at 100° C. for 10 h. The reaction mixture was cooled to room temperature, diluted with water, acidified with 2 N HCl and extracted with ethyl acetate. The combined organics were dried and concentrated and the residue was purified by flash column chromatography to give 4-cyclopropyl-2-(trifluoromethyl)quinazolin-5-ol (0.035 g, 22%) as a light brown solid.

¹H NMR (400 MHZ, CDCLl₃) δ ppm 7.72 (t, 2H), 7.02 (d, 1H), 3.65 (m, 1H), 2, 1.53 (m, 3H), 1.29 (m, 2H)

### Step 3: Synthesis of 5-(5-chloropyrimidin-2-yl)oxy-4-cyclopropyl-2-(trifluoromethyl)quinazoline

To a solution of 4-cyclopropyl-2-(trifluoromethyl)quinazolin-5-ol (0.04 g, 0.16 mmol) in isopropyl alcohol (49.3, 0.6 mL) was added potassium carbonate (0.06 g, 0.47 mmol) and 5-chloro-2-methylsulfonyl-pyrimidine (0.045 g, 0.23 mmol). The reaction mixture was heated at 50° C. for 16 h then quenched with water, extracted with ethyl acetate, dried and concentrated. The residue was purified by flash column chromatography to give 5-(5-chloropyrimidin-2-yl)oxy-4-cyclopropyl-2-(trifluoromethyl)quinazoline (0.015 g, 26%) as a yellow solid.

¹H NMR (400 MHZ, CDCl₃) δ ppm 8.48 (s, 2H) 8.12 (dd, 1H) 7.99 (t, 1H) 7.50 (dd, 1H) 3.21 (tt, 1H) 1.22-1.36 (m, 2H) 0.86-0.98 (m, 2H)

### Example 15: Synthesis of 4-(bromomethyl)-5-(5-chloropyrimidin-2-yl)oxy-2-(trifluoromethyl)quinazoline (Compound 1.048)

To a solution of 5-(5-chloropyrimidin-2-yl)oxy-4-methyl-2-(trifluoromethyl)quinazoline (0.7 g, 2.06 mmol) in carbon tetrachloride (16.4 mL) were added N-bromosuccinimide (0.549 g, 3.08 mmol) and azobisisobutyronitrile (0.10 g, 0.61 mmol). The reaction mixture was heated at 80° C. for 16 h then cooled to room temperature, quenched with a 10% solution of sodium thiosulphate and extracted with ethyl acetate. The combined organic layer was washed brine solution, dried and concentrated. The residue was purified by flash column chromatography to give 4-(bromomethyl)-5-(5-chloropyrimidin-2-yl)oxy-2-(trifluoromethyl)quinazoline (0.175 g, 20%) as an off white solid.

¹H NMR (400 MHZ, CHLOROFORM-d) δ ppm 8.58 (s, 2H), 8.21 (dd, 1H), 8.10 (t, 1H), 7.66 (dd, 1H), 5.21 (s, 2H)

### Example 16:3-[5-(5-chloropyrimidin-2-yl)oxy-2-(trifluoromethyl)quinazolin-4-yl]propanenitrile (Compound 1.046)

### Step1: Synthesis of 3-[5-methoxy-2-(trifluoromethyl)quinazolin-4-yl]prop-2-enenitrile

To a solution of 4-bromo-5-methoxy-2-(trifluoromethyl)quinazoline (0.25 g, 0.81 mmol) in toluene (5 mL) was added N,N-Diisopropylethylamine (0.3 mL, 1.79 mmol), acrylonitrile (0.06 g, 1.22 mmol) and Chloro[(tri-tert-butylphosphine)-2-(2-aminobiphenyl)] palladium(II) (0.02 g, 0.04 mmol). The reaction mixture was heated at 90° C. for 8 h then cooled to room temperature, quenched with saturated ammonium chloride solution and extracted with ethyl acetate. The combined organics were washed with brine solution, dried and concentrated. The residue was purified by flash column chromatography to give 3-[5-methoxy-2-(trifluoromethyl)quinazolin-4-yl]prop-2-enenitrile (0.12 g, 53%) as a yellow solid.

¹H NMR (400 MHZ, CDCl₃) δ ppm 8.79 (d, 1H), 7.96 (t, 1H), 7.80 (d, 1H), 7.17 (d, 1H), 7.05 (d, 1H), 4.12 (s, 3H)

### Step-2: Synthesis of 3-[5-methoxy-2-(trifluoromethyl)quinazolin-4-yl]propanenitrile

To a stirred solution of 3-[5-methoxy-2-(trifluoromethyl)quinazolin-4-yl]prop-2-enenitrile (0.12 g, 0.43 mmol) in ethanol (10.74 mL) was added 5% palladium on carbon (0.06 g). The reaction mixture was stirred at room temperature under a 1 bar hydrogen atmosphere for 8 h. The reaction mixture was filtered through a bed of celite and concentrated. The residue was purified by flash column chromatography to give 3-[5-methoxy-2-(trifluoromethyl)quinazolin-4-yl]propanenitrile (0.033 g, 27%) as an off white solid.

¹H NMR (400 MHZ, CDCl₃) δ ppm 7.91 (t, 1H) 7.78 (d, 1H), 7.13 (d, 1H), 4.09 (s, 3H) 3.91-4.00 (t, 2H), 3.04 (t, 2H).

### Step-3: Synthesis of 3-[5-hydroxy-2-(trifluoromethyl)quinazolin-4-yl]propanenitrile

To a solution of 1-dodecanethiol (0.11 g, 0.5 mmol) in N,N-dimethylformamide (0.76 mL) was added lithium tert-butoxide (0.04 g, 0.53 mmol). After stirring for 10 minutes, a solution 3-[5-methoxy-2-(trifluoromethyl)quinazolin-4-yl]propanenitrile (0.075 g, 0.26 mmol) in N, N-dimethylformamide (0.5 mL) was added. The reaction mixture was heated at 100° C. for 2.5 h then cooled to room temperature, quenched with ice cold water, acidified with the 2 N HCl and extracted with ethyl acetate. The combined organics were washed with water and brine solution, dried and concentrated. The residue was purified by flash column chromatography to give 3-[5-hydroxy-2-(trifluoromethyl)quinazolin-4-yl]propanenitrile (0.058 g, 81%) as a light brown solid.

¹H NMR (400 MHZ, CD₃OD) δ ppm 7.87 (t, 1H) 7.60 (dd, 1H) 7.17 (dd, 1H) 4.01 (t, 2H) 3.05 (t, 2H)

### Step-4: Synthesis of 3-[5-(5-chloropyrimidin-2-yl)oxy-2-(trifluoromethyl)quinazolin-4-yl]propanenitrile (Compound 1.046)

To a solution of 3-[5-hydroxy-2-(trifluoromethyl)quinazolin-4-yl]propanenitrile (0.088 g, 0.33 mmol) in isopropyl alcohol (1.32 mL) was added potassium carbonate (0.14 g, 0.99 mmol) and 5-chloro-2-methylsulfonyl-pyrimidine (0.095 g, 0.49 mmol). The reaction mixture was heated at 50° C. for 16 then cooled to room temperature, quenched with water and extracted with ethyl acetate. The combined organic layer was washed with brine solution, dried and concentrated. The residue was purified by flash column chromatography to give 3-[5-(5-chloropyrimidin-2-yl)oxy-2-(trifluoromethyl)quinazolin-4-yl]propanenitrile (0.01 g, 8%) as a brown solid.

¹H NMR (400 MHZ, CDCl₃) δ ppm 8.57 (s, 2H) 8.18 (dd, 1H) 8.05 (t, 1H) 7.54 (dd, 1H) 3.81 (t, 2H) 3.05 (t, 2H)

TABLE 1
Examples of herbicidal compounds of the present invention.

| | | ¹H NMR (400 MHz, CDCl₃ unless |
|---|---|---|
| COMPOUND | STRUCTURE | otherwise stated) |
| 1.001 | 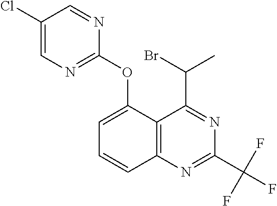 [SMILES: CC(Br)c1nc(C(F)(F)F)nc2cccc(Oc3ncc(Cl)cn3)c12] | 8.55 (s, 2H), 8.19 (dd, 1H), 8.05 (t, 1H), 7.61 (dd, 1H), 6.49 (d, 1H), 2.33-2.17 (m, 3H) |
| 1.002 | 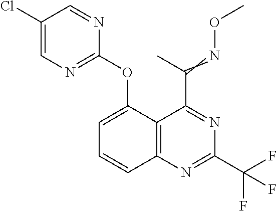 [SMILES: CON=C(C)c1nc(C(F)(F)F)nc2cccc(Oc3ncc(Cl)cn3)c12] | 8.54 (s, 2H), 8.19 (dd, 1H), 8.09 (t, 1H), 7.59 (dd, 1H), 3.31 (s, 3H), 2.32 (s, 3H) |
| 1.003 | 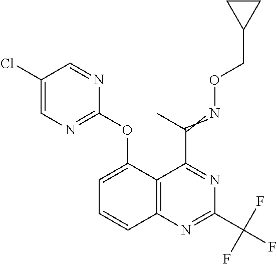 [SMILES: CC(=NOCC1CC1)c1nc(C(F)(F)F)nc2cccc(Oc3ncc(Cl)cn3)c12] | 8.50 (s, 2H), 8.19 (d, 1H), 8.08 (t, 1H), 7.59 (d, 1H), 3.50 (dd, 1H), 3.26 (dd, 1H), 2.27 (s, 3H), 0.82-0.71 (m, 1H), 0.40-0.18 (m, 2H), −0.10, −0.02 (m, 2H) |
| 1.004 | 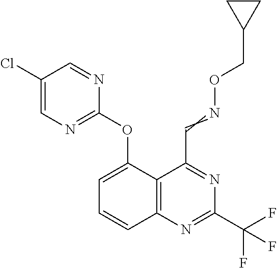 [SMILES: FC(F)(F)c1nc(C=NOCC2CC2)c2c(Oc3ncc(Cl)cn3)cccc2n1] | 8.95 (s, 1H), 8.48 (s, 2H), 8.20 (d, 1H), 8.09 (t, 1H), 7.60 (d, 1H), 3.97 (d, 2H), 1.18-1.00 (m, 1H), 0.51-0.60 (m, 2 H), 0.23-0.30 (m, 2H) |
| 1.005 | 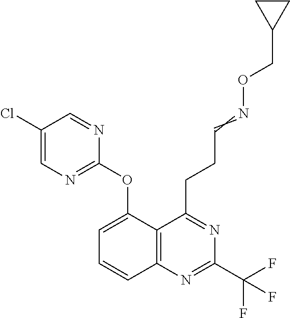 [SMILES: FC(F)(F)c1nc(CCC=NOCC2CC2)c2c(Oc3ncc(Cl)cn3)cccc2n1] | 8.54 (s, 2H), 8.15 (dd, 1H), 8.01 (t, 1H), 7.50 (dd, 1H), 6.82 (t, 1H), 3.78 (d, 2H), 3.62 (t, 2H), 2.88 (td, 2H), 1.02 (s, 1H), 0.51-0.42 (m, 2H), 0.24-0.14 (m, 2H) |
| 1.006 | 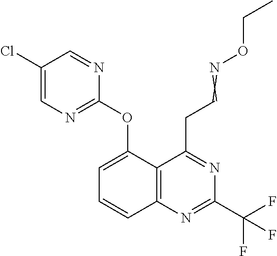 [SMILES: CCON=CCc1nc(C(F)(F)F)nc2cccc(Oc3ncc(Cl)cn3)c12] | (1:1 mixture of E/Z isomers) 8.52 (s, 2H), 8.15 (ddd, 1H), 8.02 (td, 1H), 7.72 (t, 1H), 7.51 (t, 1H), 4.42 (d, 2H), 4.04 (q, 2H), 1.16 (t, 3H) and 8.52 (s, 2H), 8.15 (ddd, 1H), 8.02 (td, 1H), 7.51 (t, 1H), 7.14 (t, 1H), 4.35 (d, 2H), 3.90 (q, 2H), 1.16 (t, 3H) |
| 1.007 | 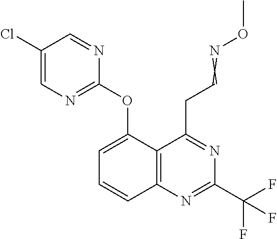 [SMILES: CON=CCc1nc(C(F)(F)F)nc2cccc(Oc3ncc(Cl)cn3)c12] | (1:1 mixture of E/Z isomers) 8.53 (s, 2H), 8.16 (dd, 1H), 8.02 (t, 1H), 7.73 (t, 1H), 7.52 (dd, 1H), 4.44 (d, 2H), 3.81 (s, 3H) and 8.53 (s, 2H), 8.14 (dd, 1H), 8.02 (t, 1H), 7.52 (dd, 1H), 7.12 (t, 1H), 4.35 (d, 2H), 3.68 (s, 3H) |
| 1.008 | 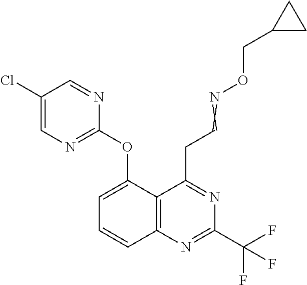 [SMILES: FC(F)(F)c1nc(CC=NOCC2CC2)c2c(Oc3ncc(Cl)cn3)cccc2n1] | (1:1 mixture of E/Z isomers) 8.52 (s, 2H), 8.20-8.11 (m, 1H), 8.02 (td, 1H), 7.76 (t, 1H), 7.52 (t, 1H), 4.44 (d, 2H), 3.80 (d, 2H), 1.08-0.92 (m, 1H), 0.54-0.41 (m, 2H), 0.20-0.13 (m, 2H) and 8.52 (s, 2H), 8.20-8.11 (m, 1H), 8.02 (td, 1H), 7.51 (t, 1H), 7.17 (t, 1H), 4.36 (d, 2H), 3.65 (d, 2H), 1.08-0.92 (m, 1H), 0.54-0.41 (m, 2H), 0.20-0.13 (m, 2H) |
| 1.009 | 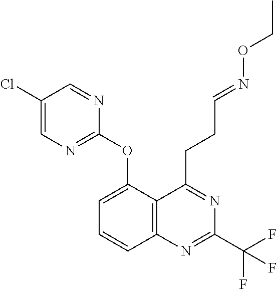 [SMILES: CCO/N=C/CCc1nc(C(F)(F)F)nc2cccc(Oc3ncc(Cl)cn3)c12] | 8.53 (s, 2H), 8.15 (dd, 1H), 8.01 (t, 1H), 7.47-7.54 (m, 2H), 3.93-4.01 (m, 2H), 3.57-3.65 (m, 2H), 2.72-2.83 (m, 2H), 1.17 (t, 3H) |
| 1.010 | 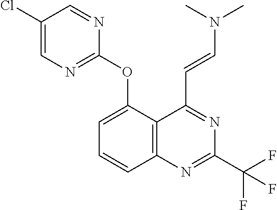 [SMILES: CN(C)/C=C/c1nc(C(F)(F)F)nc2cccc(Oc3ncc(Cl)cn3)c12] | 8.44 (s, 2H), 8.32 (d, 1H), 7.84 (d, 1H), 7.76 (t, 1H), 7.23 (t, 1H), 6.27 (d, 1H), 3.16 (br s, 3H), 2.90 (br s, 3H), |
| 1.011 | 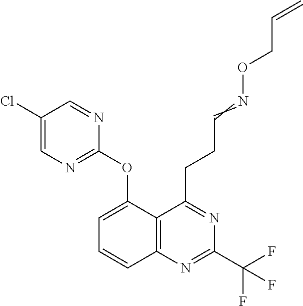 [SMILES: C=CCON=CCCc1nc(C(F)(F)F)nc2cccc(Oc3ncc(Cl)cn3)c12] | 8.53 (s, 2H), 8.14 (d, 1H), 8.00 (t, 1 H), 7.56 (t, 1H), 7.49 (dd, 1H), 5.84-5.94 (m, 1H), 5.13-5.25 (m, 2H), 4.42 (dt, 2H), 3.61 (t, 2H), 2.79 (td, 2H) |
| 1.012 | 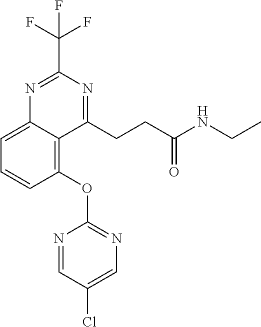 [SMILES: CCNC(=O)CCc1nc(C(F)(F)F)nc2cccc(Oc3ncc(Cl)cn3)c12] | 8.83 (s, 2H), 8.12-8.22 (m, 2H), 7.78- 7.84 (m, 2H), 3.56 (t, 2H), 2.99 (dd, 2 H), 2.56-2.63 (m, 2H), 0.95 (t, 3H) |
| 1.013 | 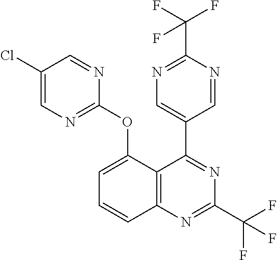 [SMILES: FC(F)(F)c1ncc(-c2nc(C(F)(F)F)nc3cccc(Oc4ncc(Cl)cn4)c23)cn1] | 8.93 (s, 2H), 8.34 (dd, 1H), 8.23 (s, 2H), 8.20 (t, 1H), 7.64 (dd, 1H), |
| 1.014 | 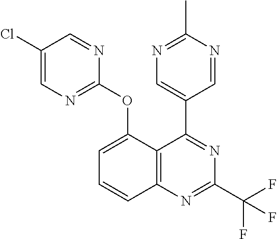 [SMILES: Cc1ncc(-c2nc(C(F)(F)F)nc3cccc(Oc4ncc(Cl)cn4)c23)cn1] | 8.72 (s, 2H), 8.30 (dd, 1H), 8.24 (s, 2H), 8.16 (t, 1H), 7.57 (dd, 1H), 2.80 (s, 3H) |
| 1.015 | 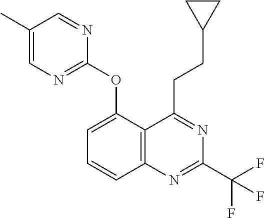 [SMILES: Cc1cnc(Oc2cccc3nc(C(F)(F)F)nc(CCC4CC4)c23)nc1] | 8.42 (s, 2H), 8.09 (dd, 1H), 7.96 (t, 1H), 7.44 (dd, 1H), 3.44-3.51 (m, 2H), 2.31 (s, 3H), 1.64-1.76 (m, 2H) 0.74-0.78 (m, 1H), 0.28-0.39 (m, 2H), 0.02 (q, 2H) |
| 1.016 | 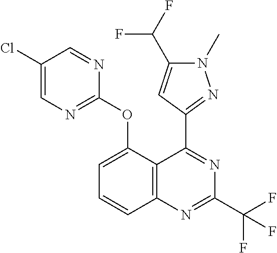 [SMILES: Cn1nc(-c2nc(C(F)(F)F)nc3cccc(Oc4ncc(Cl)cn4)c23)cc1C(F)F] | 8.29 (s, 2H), 8.23 (dd, 1H), 8.06-8.13 (m, 1H), 7.59 (dd, 1H), 6.71 (d, 1H), 6.70 (t, 1H), 3.87 (s, 3H) |
| 1.018 | 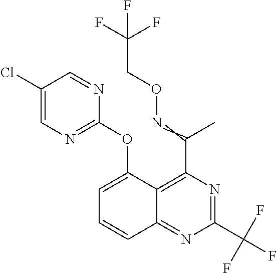 [SMILES: CC(=NOCC(F)(F)F)c1nc(C(F)(F)F)nc2cccc(Oc3ncc(Cl)cn3)c12] | (major isomer) 8.49 (s, 2H), 8.21 (dd, 1H), 8.07-8.15 (m, 1H), 7.60 (dd, 1H), 4.20 (q, 2H), 2.40 (s, 3H). (minor isomer) 8.52 (s, 2H) 8.21 (dd, 1H), 8.07-8.15 (m, 1H), 7.60 (dd, 1H), 3.96-4.16 (m, 2H), 2.33 (s, 3H) |
| 1.019 | 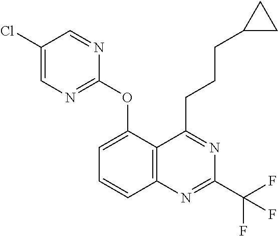 [SMILES: FC(F)(F)c1nc(CCCC2CC2)c2c(Oc3ncc(Cl)cn3)cccc2n1] | 8.53 (s, 2H), 8.13 (dd, 1H), 7.98 (t, 1H), 7.46 (dd, 1H), 3.32-3.45 (m, 2H), 1.78- 1.95 (m, 2H), 1.11-1.35 (m, 2H), 0.53- 0.65 (m, 1H), 0.28-0.38 (m, 2H), −0.10- 0.01 (m, 2H) |
| 1.021 | 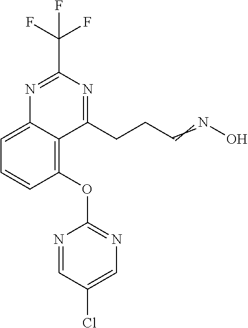 [SMILES: ON=CCCc1nc(C(F)(F)F)nc2cccc(Oc3ncc(Cl)cn3)c12] | 8.54 (s, 2H), 8.16 (d, 1H), 8.02 (t, 1H), 7.51 (d, 1H), 6.92 (t, 1H), 3.65 (t, 2H), 2.91 (td, 2H) one OH proton missing |
| 1.022 | 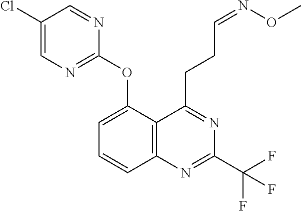 [SMILES: CO/N=C\CCc1nc(C(F)(F)F)nc2cccc(Oc3ncc(Cl)cn3)c12] | 8.54 (s, 2H), 8.15 (dd, 1H), 8.01 (t, 1H), 7.50 (dd, 1H), 6.81 (t, 1H), 3.77 (s, 3H), 3.56-3.64, (m, 2H), 2.80-2.87 (m, 2H) |
| 1.023 | 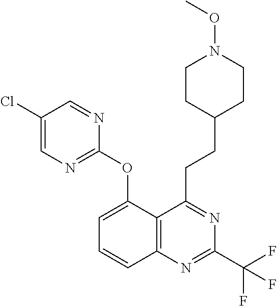 [SMILES: CON1CCC(CCc2nc(C(F)(F)F)nc3cccc(Oc4ncc(Cl)cn4)c23)CC1] | 8.53 (s, 2 H), 8.13 (d, 1 H), 7.98 (t, 1 H), 7.46 (d, 1 H), 3.52 (s, 3 H), 3.31-3.39 (m, 4 H), 2.23-2.29 (m, 2 H), 1.64-1.72 (m, 4H), 1.20-1.30 (m, 3 H) |
| 1.024 | 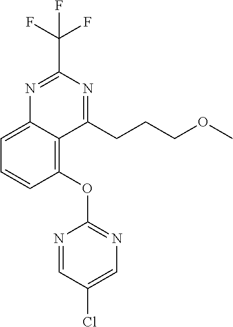 [SMILES: COCCCc1nc(C(F)(F)F)nc2cccc(Oc3ncc(Cl)cn3)c12] | 8.53 (s, 2 H), 8.13 (dd, 1 H), 7.99 (t, 1 H), 7.47 (dd, 1 H), 3.40-3.47 (m, 4 H), 3.24 (s, 3 H), 2.03-2.11 (m, 2 H) |
| 1.025 | 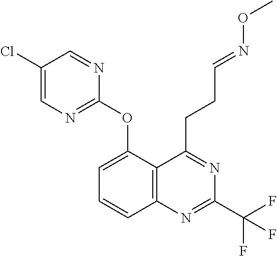 [SMILES: CO/N=C/CCc1nc(C(F)(F)F)nc2cccc(Oc3ncc(Cl)cn3)c12] | 8.54 (s, 2 H), 8.15 (d, 1 H), 8.01 (t, 1 H), 7.47-7.54 (m, 2 H), 3.74 (s, 3 H), 3.62 (t, 2 H), 2.77 (td, 2 H) |
| 1.026 | 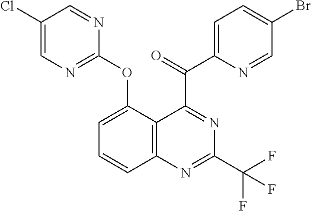 [SMILES: O=C(c1ccc(Br)cn1)c1nc(C(F)(F)F)nc2cccc(Oc3ncc(Cl)cn3)c12] | 8.41 (d, 1 H), 8.36 (s, 2 H), 8.25 (d, 1 H), 8.05-8.16 (m, 3 H), 7.56 (d, 1 H) |
| 1.027 | 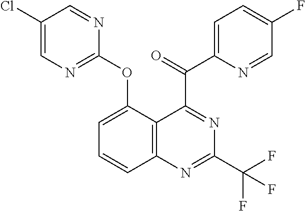 [SMILES: O=C(c1ccc(F)cn1)c1nc(C(F)(F)F)nc2cccc(Oc3ncc(Cl)cn3)c12] | 8.38 (s, 2 H), 8.22-8.28 (m, 1 H), 8.24 (t, 2 H), 8.13 (t, 1 H), 7.63 (td, 1 H), 7.56 (d, 1 H) |
| 1.028 | 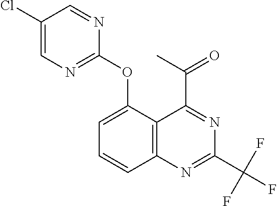 [SMILES: CC(=O)c1nc(C(F)(F)F)nc2cccc(Oc3ncc(Cl)cn3)c12] | 8.52 (s, 2 H), 8.19-8.23 (m, 1 H) 8.11- 8.18 (m, 1 H), 7.63-7.68 (m, 1 H), 2.78 (s, 3 H) |
| 1.029 | 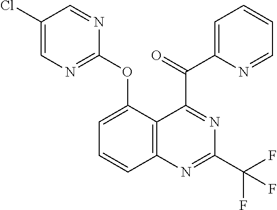 [SMILES: O=C(c1ccccn1)c1nc(C(F)(F)F)nc2cccc(Oc3ncc(Cl)cn3)c12] | 8.36-8.38 (m, 1 H), 8.33 (s, 2 H), 8.23- 8.27 (m, 2 H), 8.12 (t, 1 H), 7.92 (td, 1 H), 7.55 (d, 1 H), 7.43 (ddd, 1 H) |
| 1.030 | 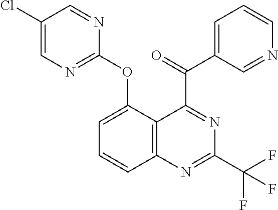 [SMILES: O=C(c1cccnc1)c1nc(C(F)(F)F)nc2cccc(Oc3ncc(Cl)cn3)c12] | 8.88 (s, 1 H), 8.82 (br d, 1 H), 8.39 (s, 2 H), 8.30 (dd, 1 H), 8.19 (t, 1 H), 8.13 (dt, 1 H), 7.62 (dd, 1 H), 7.44 (dd, 1 H) |
| 1.031 | 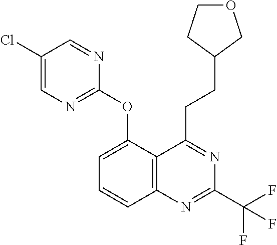 [SMILES: FC(F)(F)c1nc(CCC2CCOC2)c2c(Oc3ncc(Cl)cn3)cccc2n1] | 8.54 (s, 2 H), 8.14 (dd, 1 H), 8.00 (t, 1 H), 7.48 (dd, 1 H), 3.79-3.88 (m, 2 H), 3.71- 3.76 (m, 1 H), 3.32-3.41 (m, 3 H), 2.21-2.29 (m, 1 H), 1.99-2.07 (m, 1 H), 1.86-1.93 (m, 2 H), 1.49-1.57 (m, 1 H) |
| 1.032 | 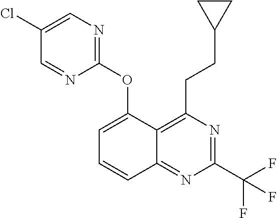 [SMILES: FC(F)(F)c1nc(CCC2CC2)c2c(Oc3ncc(Cl)cn3)cccc2n1] | 8.53 (s, 2 H), 8.13 (dd, 1 H), 7.98 (t, 1 H), 7.46 (dd, 1 H), 3.36-3.52 (m, 2 H), 1.63- 1.75 (m, 2 H), 0.71-0.80 (m, 1 H), 0.33- 0.40 (m, 2 H), 0.01 (q, 2 H) |
| 1.033 | 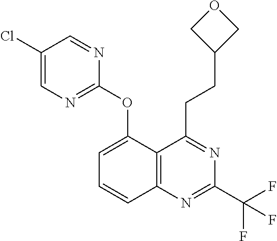 [SMILES: FC(F)(F)c1nc(CCC2COC2)c2c(Oc3ncc(Cl)cn3)cccc2n1] | 8.56 (s, 2H), 8.17 (dd, 1 H), 8.03 (t, 1 H), 7.52 (dd, 1 H), 4.76 (dd, 2 H), 4.40 (t, 2 H), 3.28-3.35 (m, 2 H), 3.10 (t, 1 H), 2.23 (q, 2 H) |
| 1.034 | 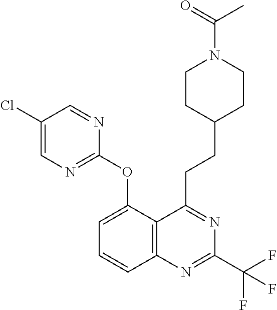 [SMILES: CC(=O)N1CCC(CCc2nc(C(F)(F)F)nc3cccc(Oc4ncc(Cl)cn4)c23)CC1] | 8.53 (s, 2 H), 8.12 (dd, 1 H), 7.98 (t, 1 H), 7.46 (dd, 1 H), 4.51-4.61 (m, 1 H), 3.77 (br d, 1 H), 3.28-3.45 (m, 2 H), 2.94- 3.02 (m, 1 H), 2.48 (td, 1 H), 2.02-2.15 (m, 3 H), 1.69-1.80 (m, 3 H), 1.65 (br d, 1 H), 1.54 (ddd, 1 H), 1.01-1.18 (m, 2 H) |
| 1.035 | 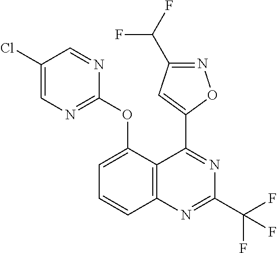 [SMILES: FC(F)c1cc(-c2nc(C(F)(F)F)nc3cccc(Oc4ncc(Cl)cn4)c23)on1] | 8.39 (s, 2 H), 8.30 (d, 1 H), 8.19 (t, 1 H), 7.66 (d, 1 H), 6.86 (s, 1 H), 6.80 (t, 1 H) |
| 1.036 | 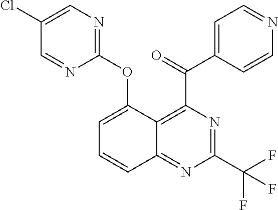 [SMILES: O=C(c1ccncc1)c1nc(C(F)(F)F)nc2cccc(Oc3ncc(Cl)cn3)c12] | 8.81 (d, 2 H), 8.40 (s, 2 H), 8.30 (d, 1 H), 8.19 (t, 1 H), 7.55-7.63 (m, 3 H) |
| 1.037 | 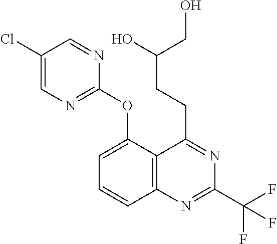 [SMILES: OCC(O)CCc1nc(C(F)(F)F)nc2cccc(Oc3ncc(Cl)cn3)c12] | 8.56 (s, 2 H), 8.16 (dd, 1 H), 8.03 (t, 1 H), 7.58 (dd, 1 H), 3.66 (d, 1 H), 3.45-3.62 (m, 4 H), 2.02-2.09 (m, 2 H) (2-OH proton missing) |
| 1.038 | 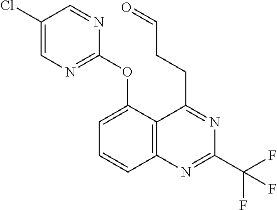 [SMILES: O=CCCc1nc(C(F)(F)F)nc2cccc(Oc3ncc(Cl)cn3)c12] | 9.97 (s, 1 H), 8.55 (s, 2 H), 8.15 (dd, 1 H), 8.02 (t, 1 H), 7.52 (dd, 1 H), 3.73-3.80 (m, 2 H), 3.08 (t, 2 H) |
| 1.039 | 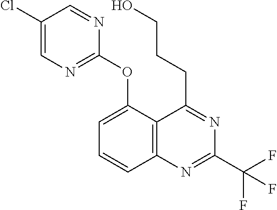 [SMILES: OCCCc1nc(C(F)(F)F)nc2cccc(Oc3ncc(Cl)cn3)c12] | 8.67 (s, 2 H), 8.04-8.13 (m, 2 H), 7.66 (s, 1 H), 3.63 (t, 2 H), 3.39-3.51 (m, 3 H), 2.01 (br dd, 2 H) |
| 1.040 | 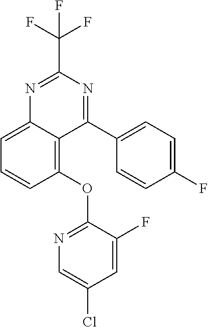 [SMILES: Fc1ccc(-c2nc(C(F)(F)F)nc3cccc(Oc4ncc(Cl)cc4F)c23)cc1] | 8.25 (d, 1 H), 8.10 (t, 1 H), 7.58 (d, 1 H), 7.52 (d, 1 H), 7.44 (dd, 2 H), 7.27 (d, 1 H), 7.00 (t, 2 H) |
| 1.041 | 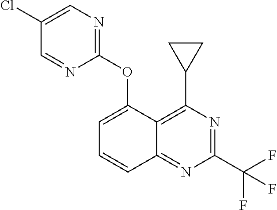 [SMILES: FC(F)(F)c1nc(C2CC2)c2c(Oc3ncc(Cl)cn3)cccc2n1] | 8.48 (s, 2 H), 8.12 (dd, 1 H), 7.99 (t, 1 H), 7.50 (dd, 1 H), 3.21 (tt, 1 H), 1.22-1.36 (m, 2 H), 0.86-0.98 (m, 2 H) |
| 1.042 | 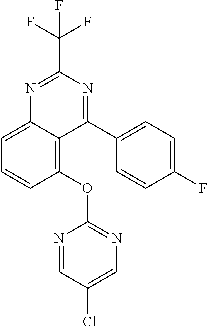 [SMILES: Fc1ccc(-c2nc(C(F)(F)F)nc3cccc(Oc4ncc(Cl)cn4)c23)cc1] | 8.29 (dd, 1 H), 8.19 (s, 2 H), 8.13 (dd, 1 H), 7.57 (dd, 1 H), 7.43 (t, 2 H), 7.04 (t, 2 H) |
| 1.043 | 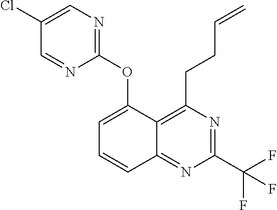 [SMILES: C=CCCc1nc(C(F)(F)F)nc2cccc(Oc3ncc(Cl)cn3)c12] | |
| 1.044 | 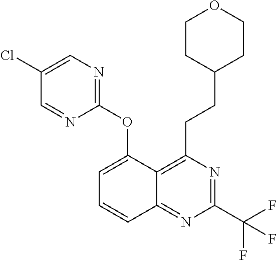 [SMILES: FC(F)(F)c1nc(CCC2CCOCC2)c2c(Oc3ncc(Cl)cn3)cccc2n1] | 8.54 (s, 2 H), 8.14 (dd, 1 H), 7.99 (t, 1 H), 7.46 (dd, 1 H), 3.94 (dd, 2 H), 3.30-3.41 (m, 4 H), 1.69-1.76 (m, 2 H), 1.54-1.56 (m, 1 H), 1.20-1.33 (m, 4 H) |
| 1.045 | 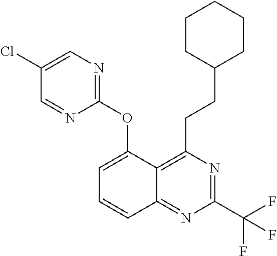 [SMILES: FC(F)(F)c1nc(CCC2CCCCC2)c2c(Oc3ncc(Cl)cn3)cccc2n1] | 8.53 (s, 2 H), 8.09-8.16 (m, 1 H), 7.97 (t, 1 H), 7.41-7.49 (m, 1 H), 3.31-3.41 (m, 2 H), 1.57-1.65 (m, 6 H), 1.10- 1.33 (m, 4 H), 0.78-0.93 (m, 3H H) |
| 1.046 | 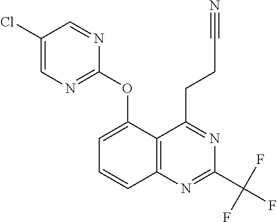 [SMILES: N#CCCc1nc(C(F)(F)F)nc2cccc(Oc3ncc(Cl)cn3)c12] | 8.57 (s, 2 H), 8.18 (dd, 1 H), 8.05 (t, 1 H), 7.54 (dd, 1 H), 3.78-3.85 (m, 2 H), 3.05 (t, 2 H) |
| 1.047 | 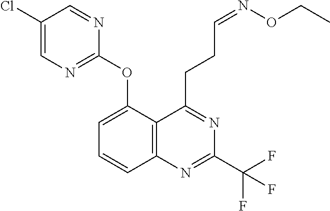 [SMILES: CCO/N=C\CCc1nc(C(F)(F)F)nc2cccc(Oc3ncc(Cl)cn3)c12] | 8.54 (s, 2H), 8.15 (d, 1H), 8.01 (t, 1H), 7.50 (d, 1H), 6.81 (t, 1H), 4.01 (q, 2H), 3.61 (t, 2H), 2.77-2.93 (m, 2H), 1.16 (t, 3H) |
| 1.048 | 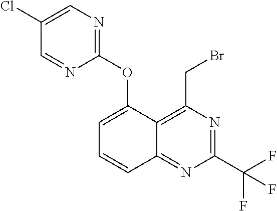 [SMILES: FC(F)(F)c1nc(CBr)c2c(Oc3ncc(Cl)cn3)cccc2n1] | 8.58 (s, 2 H), 8.21 (dd, 1 H), 8.10 (t, 1 H), 7.66 (dd, 1 H), 5.21 (s, 2 H) |
| 1.049 | 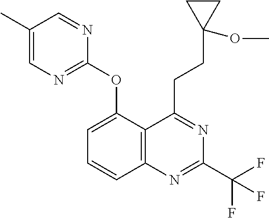 | |
| 1.050 | 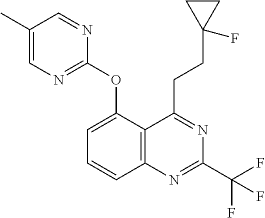 [SMILES: Cc1cnc(Oc2cccc3nc(C(F)(F)F)nc(CCC4(F)CC4)c23)nc1] | |
| 1.051 | 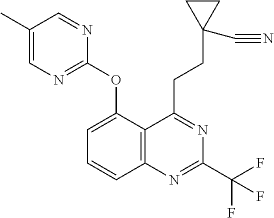 [SMILES: Cc1cnc(Oc2cccc3nc(C(F)(F)F)nc(CCC4(C#N)CC4)c23)nc1] | |
| 1.052 | 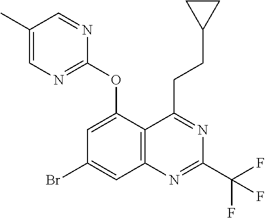 [SMILES: Cc1cnc(Oc2cc(Br)cc3nc(C(F)(F)F)nc(CCC4CC4)c23)nc1] | |
| 1.053 | 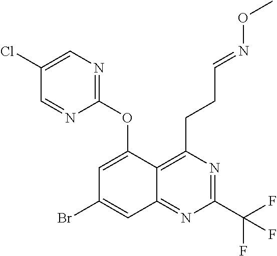 [SMILES: CO/N=C/CCc1nc(C(F)(F)F)nc2cc(Br)cc(Oc3ncc(Cl)cn3)c12] | |
| 1.054 | 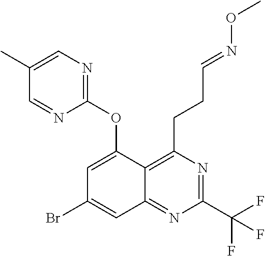 | |
| 1.055 | 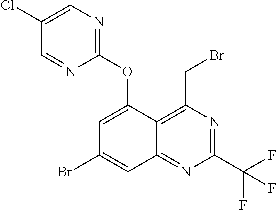 [SMILES: FC(F)(F)c1nc(CBr)c2c(Oc3ncc(Cl)cn3)cc(Br)cc2n1] | |
| 1.056 | 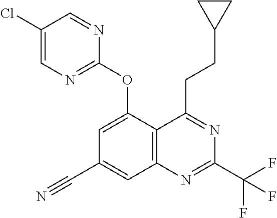 [SMILES: N#Cc1cc(Oc2ncc(Cl)cn2)c2c(CCC3CC3)nc(C(F)(F)F)nc2c1] | |
| 1.057 | 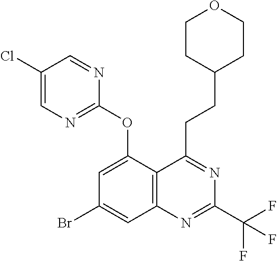 [SMILES: FC(F)(F)c1nc(CCC2CCOCC2)c2c(Oc3ncc(Cl)cn3)cc(Br)cc2n1] | |
| 1.058 | 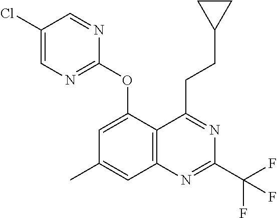 [SMILES: Cc1cc(Oc2ncc(Cl)cn2)c2c(CCC3CC3)nc(C(F)(F)F)nc2c1] | |
| 1.059 | 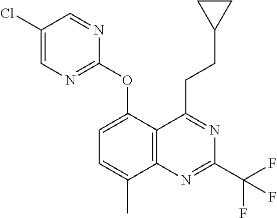 [SMILES: Cc1ccc(Oc2ncc(Cl)cn2)c2c(CCC3CC3)nc(C(F)(F)F)nc12] | 8.51 (s, 2 H), 7.81 (dd, 1 H), 7.34 (d, 1 H), 3.37-3.43 (m, 2 H), 2.80 (s, 3 H), 1.63-1.72 (m, 2 H), 0.70-0.78 (m, 1 H), 0.32-0.38 (m, 2 H), −0.04-0.02 (m, 2 H) |
| 1.060 | 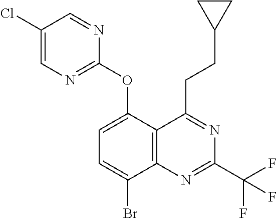 [SMILES: FC(F)(F)c1nc(CCC2CC2)c2c(Oc3ncc(Cl)cn3)ccc(Br)c2n1] | 8.52 (s, 2 H), 8.28 (d, 1 H), 7.35 (d, 1 H), 3.41-3.49 (m, 2 H), 1.64-1.73 (m, 2 H), 0.69-0.79 (m, 1 H), 0.31-0.40 (m, 2 H), −0.05-0.04 (m, 2 H) |
| 1.061 | 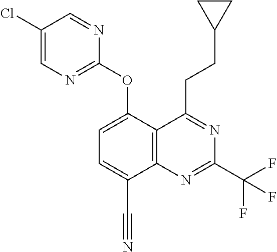 [SMILES: N#Cc1ccc(Oc2ncc(Cl)cn2)c2c(CCC3CC3)nc(C(F)(F)F)nc12] | |
| 1.062 | 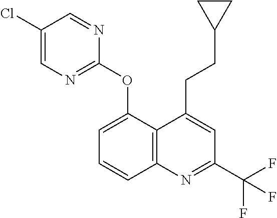 [SMILES: FC(F)(F)c1cc(CCC2CC2)c2c(Oc3ncc(Cl)cn3)cccc2n1] | |
| 1.064 | 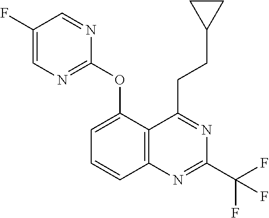 [SMILES: Fc1cnc(Oc2cccc3nc(C(F)(F)F)nc(CCC4CC4)c23)nc1] | 8.47 (s, 2 H), 8.13 (dd, 1 H), 7.99 (t, 1 H), 7.46 (dd, 1 H), 3.44-3.51, (m, 2 H), 1.63- 1.79 (m, 2 H), 0.72-0.86 (m, 1 H), 0.33- 0.40 (m, 2 H), −0.02-0.03 (m, 2 H) |
| 1.065 | 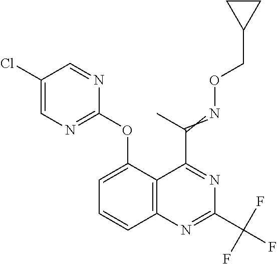 [SMILES: CC(=NOCC1CC1)c1nc(C(F)(F)F)nc2cccc(Oc3ncc(Cl)cn3)c12] | (Major isomer) 8.47 (s, 2 H), 8.19 (dd, 1 H), 8.08 (t, 1 H), 7.57 (dd, 1 H), 3.50 (d, 2 H), 2.32 (s, 3 H), 0.83-1.01 (m, 1 H), 0.47-0.53 (m, 2 H), 0.14-0.19 (m, 2 H) (Minor isomer) 8.50 (s, 2 H), 8.19 (d, 1 H), 8.08 (t, 1 H), 7.59 (d, 1 H), 3.50 (dd, 1 H), 3.26 (dd, 1 H), 2.27 (s, 3 H), 0.71- 0.82 (m, 1 H), 0.18-0.40 (m, 2 H), −0.10- 0.02 (m, 2 H) |
| 1.066 | 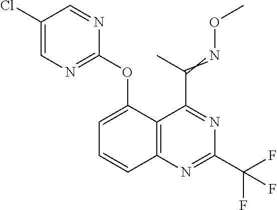 [SMILES: CON=C(C)c1nc(C(F)(F)F)nc2cccc(Oc3ncc(Cl)cn3)c12] | (Major isomer) 8.49 (s, 2 H), 8.20 (dd, 1 H), 8.10 (t, 1 H), 7.58 (dd, 1 H), 3.55 (s, 3 H), 2.30 (s, 3 H) (Minor isomer) 8.54 (s, 2 H), 8.19 (dd, 1 H), 8.09 (t, 1 H), 7.59 (dd, 1 H), 3.31 (s, 3 H), 2.32 (s, 3 H) |
| 1.067 | 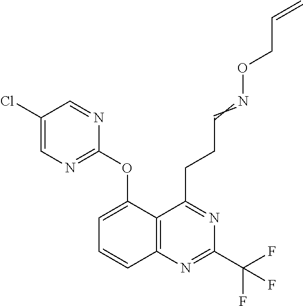 [SMILES: C=CCON=CCCc1nc(C(F)(F)F)nc2cccc(Oc3ncc(Cl)cn3)c12] | (Major isomer) 8.53 (s, 2 H), 8.14 (d, 1 H), 8.00 (t, 1 H), 7.56 (t, 1 H), 7.49 (dd, 1 H), 5.84-5.94 (m, 1 H), 5.13-5.25 (m, 2 H), 4.42 (dt, 2 H), 3.61 (t, 2 H), 2.79 (td, 2 H) (Minor isomer) 8.53 (s, 2 H), 8.15 (d, 1 H), 8.01 (t, 1 H), 7.50 (d, 1 H), 6.86 (t, 1 H), 5.88 (m, 1 H), 5.13-5.25 (m, 2 H), 4.47 (dt, 2 H), 3.62 (t, 2 H), 2.88 (td, 2 H) |
| 1.068 | 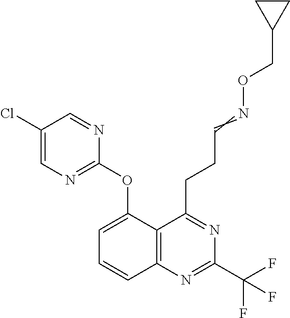 [SMILES: FC(F)(F)c1nc(CCC=NOCC2CC2)c2c(Oc3ncc(Cl)cn3)cccc2n1] | (Major isomer) 8.53 (s, 2 H), 8.15 (d, 1 H), 8.01 (t, 1 H), 7.55 (m, 2 H), 7.49 (br d, 1 H), 3.74 (br d, 2 H), 3.62 (br t, 2 H), 2.79 (br d, 2 H), 1.00-1.11 (m, 1 H), 0.51 (br d, 2 H), 0.21-0.22 (m, 1 H) (Minor isomer) 8.54 (s, 2 H), 8.15 (dd, 1 H), 8.01 (t, 1 H), 7.50 (dd, 1 H), 6.82 (t, 1 H), 3.78 (d, 2 H), 3.62 (t, 2 H), 2.88 (td, 2 H), 1.02 (s, 1 H), 0.42-0.51 (m, 2 H), 0.14- 0.24 (m, 2 H) |
| 1.069 | 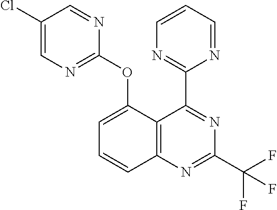 [SMILES: FC(F)(F)c1nc(-c2ncccn2)c2c(Oc3ncc(Cl)cn3)cccc2n1] | 8.69 (d, 2 H), 8.30 (s, 2 H), 8.27 (dd, 1 H), 8.13 (t, 1 H), 7.55 (dd, 1 H), 7.38 (t, 1 H) |
| 1.070 | 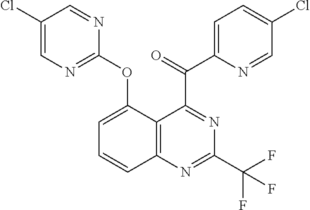 [SMILES: O=C(c1ccc(Cl)cn1)c1nc(C(F)(F)F)nc2cccc(Oc3ncc(Cl)cn3)c12] | 8.36 (s, 2 H), 8.31 (d, 1 H), 8.10-8.28 (m, 3 H), 7.91 (dd, 1 H), 7.55 (d, 1 H) |
| 1.071 | 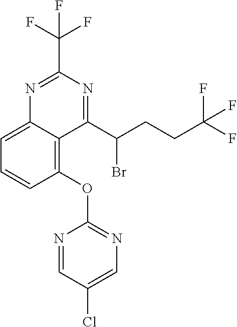 [SMILES: FC(F)(F)CCC(Br)c1nc(C(F)(F)F)nc2cccc(Oc3ncc(Cl)cn3)c12] | 8.55 (s, 2 H), 8.20 (d, 1 H), 8.07 (t, 1 H), 7.63 (d, 1 H), 6.35 (dd, 1 H), 2.66-2.86 (m, 2 H), 2.25-2.49 (m, 2 H) |

### Biological Examples

Seeds of a variety of test species are sown in standard soil in pots Amaranthus retoflexus (AMARE), Amaranthus palmeri (AMAPA), Echinochloa crus-galli (ECHCG), Ipomoea hederacea (IPOHE), Setaria faberi (SETFA). After cultivation for one day (pre-emergence) or after 8 days cultivation (post-emergence) under controlled conditions in a glasshouse (at 24/16° C., day/night; 14 hours light; 65% humidity), the plants are sprayed with an aqueous spray solution derived from the formulation of the technical active ingredient in acetone/water (50:50) solution containing 0.5% Tween 20 (polyoxyethelyene sorbitan monolaurate, CAS RN 9005-64-5). Compounds are applied at 250 g/ha unless otherwise stated. The test plants are then grown in a glasshouse under controlled conditions in a glasshouse (at 24/16° C., day/night; 14 hours light; 65% humidity) and watered twice daily. After 13 days for pre and post-emergence, the test is evaluated for the percentage damage caused to the plant. The biological activities are shown in the following table on a five-point scale (5=81-100%; 4=61-80%; 3=41-60%; 2=21-40%; 1=0-20%).

TABLE B1
Post-emergence Test

| Compound | AMARE | AMAPA | ECHCG | IPOHE | SETFA |
|---|---|---|---|---|---|
| 1.001 | 3 | 3 | 1 | 3 | 1 |
| 1.003 | 1 | 1 | 1 | 1 | 1 |
| 1.005 | 4 | 5 | 4 | 2 | 4 |
| 1.006 | 3 | 2 | 1 | 1 | 1 |
| 1.007 | 2 | 2 | 1 | 1 | 1 |
| 1.008 | 4 | 4 | 2 | 1 | 1 |
| 1.009 | 5 | 5 | 5 | 5 | 5 |
| 1.010 | 3 | 3 | 1 | 1 | 1 |
| 1.011 | 5 | 4 | 4 | 2 | 4 |
| 1.012 | 2 | 3 | 1 | 1 | 1 |
| 1.013 | 2 | 2 | 1 | 1 | 1 |
| 1.014 | 1 | 1 | 1 | 1 | 1 |
| 1.015 | 4 | 2 | 4 | 3 | 5 |
| 1.016 | 2 | 2 | 1 | 2 | 1 |
| 1.018 | 2 | 2 | 1 | 2 | 1 |
| 1.019 | 5 | 5 | 2 | 3 | 5 |
| 1.021 | 5 | 4 | 2 | 2 | 2 |
| 1.022 | 4 | 4 | 4 | 3 | 4 |
| 1.023 | 4 | 4 | 1 | 3 | 1 |
| 1.024 | 4 | 3 | 5 | 3 | 4 |
| 1.025 | 5 | 4 | 5 | 4 | 5 |
| 1.026 | 1 | 2 | 1 | 1 | 1 |
| 1.027 | 2 | 1 | 1 | 1 | 1 |
| 1.028 | 4 | 4 | 1 | 3 | 2 |
| 1.029 | 1 | 1 | 1 | 1 | 1 |
| 1.030 | 5 | 5 | 2 | 2 | 2 |
| 1.031 | 5 | 4 | 5 | 5 | 5 |
| 1.032 | 5 | 4 | 5 | 4 | 5 |
| 1.033 | 4 | 2 | 5 | 5 | 5 |
| 1.034 | 5 | 4 | 4 | 2 | 5 |
| 1.035 | 4 | 4 | 1 | 1 | 2 |
| 1.036 | 3 | 3 | 1 | 1 | 1 |
| 1.037 | 3 | 3 | 1 | 1 | 1 |
| 1.038 | 4 | 3 | 1 | 3 | 1 |
| 1.039 | 4 | 3 | 1 | 3 | 1 |
| 1.040 | 2 | — | 1 | 2 | 1 |
| 1.041 | 5 | 5 | 3 | 2 | 5 |
| 1.042 | 3 | — | 1 | 3 | 1 |
| 1.043 | 5 | — | 4 | 3 | 4 |
| 1.044 | 5 | — | 5 | 4 | 5 |
| 1.045 | 4 | 4 | 2 | 3 | 2 |
| 1.046 | — | 5 | 2 | 4 | 2 |
| 1.047 | 5 | 4 | 5 | 5 | 5 |
| 1.048 | 5 | 4 | 2 | 2 | 4 |
| 1.059 | 2 | 2 | 1 | 3 | 1 |
| 1.060 | 3 | 3 | 1 | 3 | 2 |
| 1.064 | 5 | 4 | 4 | 4 | — |
| 1.065 | 2 | 2 | 1 | 1 | 1 |
| 1.066 | 1 | 2 | 1 | 1 | 1 |
| 1.067 | 4 | 5 | 4 | 3 | 5 |
| 1.068 | 5 | 5 | 5 | 1 | 5 |
| 1.069 | 1 | 1 | 1 | 1 | 1 |
| 1.070 | 2 | 1 | 1 | 1 | 1 |
| 1.071 | 5 | 5 | 5 | 3 | 4 |

TABLE B2
Pre-emergence Test

| Compound | AMARE | AMAPA | ECHCG | IPOHE | SETFA |
|---|---|---|---|---|---|
| 1.001 | 2 | 3 | 1 | 1 | 2 |
| 1.003 | 1 | 1 | 1 | 1 | 1 |
| 1.005 | 3 | 5 | 4 | 1 | 5 |
| 1.006 | 1 | 3 | 1 | 1 | 1 |
| 1.007 | 1 | 1 | 1 | 1 | 1 |
| 1.008 | 1 | 2 | 1 | 1 | 1 |
| 1.009 | 5 | 5 | 5 | 1 | 5 |
| 1.010 | 2 | 2 | 1 | 1 | 1 |
| 1.011 | 5 | 5 | 4 | 2 | 5 |
| 1.012 | 1 | 2 | 1 | 1 | 1 |
| 1.013 | 1 | 1 | 1 | 1 | 1 |
| 1.014 | 2 | 2 | 1 | 1 | 1 |
| 1.015 | 5 | 5 | 5 | 3 | 5 |
| 1.016 | 1 | 2 | 1 | 1 | 1 |
| 1.018 | 1 | 1 | 1 | 1 | 1 |
| 1.019 | 4 | 5 | 4 | 1 | 5 |
| 1.021 | 4 | 4 | 3 | 1 | 3 |
| 1.022 | 5 | 5 | 5 | 4 | 5 |
| 1.023 | 4 | 4 | 2 | 1 | 1 |
| 1.024 | 5 | 5 | 5 | 5 | 4 |
| 1.025 | 5 | 5 | 5 | 4 | 5 |
| 1.026 | 4 | 4 | 1 | 1 | 1 |
| 1.027 | 2 | 2 | 1 | 1 | 1 |
| 1.028 | 5 | 5 | 2 | 1 | 3 |
| 1.029 | 1 | 1 | 1 | 1 | 1 |
| 1.030 | 5 | 5 | 4 | 1 | 4 |
| 1.031 | 5 | 5 | 5 | 5 | 5 |
| 1.032 | 5 | 5 | 5 | 1 | 5 |
| 1.033 | 4 | 5 | 5 | 5 | 5 |
| 1.034 | 2 | 3 | 2 | 1 | 4 |
| 1.035 | 5 | 5 | 2 | 1 | 3 |
| 1.036 | 3 | 4 | 3 | 1 | 3 |
| 1.037 | 1 | 1 | 1 | 1 | 1 |
| 1.038 | 2 | 1 | 1 | 1 | 1 |
| 1.039 | 3 | 2 | 2 | 1 | 2 |
| 1.040 | 1 | — | 1 | 1 | 1 |
| 1.041 | 2 | 5 | 3 | 1 | 5 |
| 1.042 | 1 | — | 1 | 1 | 1 |
| 1.043 | 4 | — | 5 | 1 | 5 |
| 1.044 | 5 | — | 5 | 5 | 5 |
| 1.045 | 3 | 5 | 4 | 1 | 4 |
| 1.046 | 5 | 5 | 5 | 5 | 5 |
| 1.047 | 4 | 4 | 5 | 2 | 5 |
| 1.048 | 5 | 5 | 3 | 1 | 4 |
| 1.059 | 1 | 1 | 1 | 1 | 1 |
| 1.060 | 1 | 2 | 1 | 1 | 2 |
| 1.064 | 5 | 5 | 4 | 5 | — |
| 1.065 | 1 | 1 | 1 | 1 | 1 |
| 1.066 | 1 | 4 | 1 | 1 | 1 |
| 1.067 | 3 | 5 | 5 | 1 | 5 |
| 1.068 | 4 | 5 | 3 | 1 | 5 |
| 1.069 | 1 | 1 | 1 | 1 | 1 |
| 1.070 | 5 | 5 | 1 | 1 | 1 |
| 1.071 | 5 | 5 | 5 | 1 | 5 |

1. A compound of Formula (I): 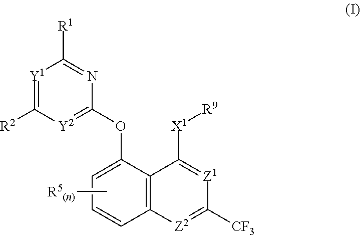
or an agronomically acceptable salt thereof,
wherein
Y¹ is N or CR³;
Y² is N or CR⁴;
with the proviso that Y¹ and Y² are not both N;
R¹ is selected from the group consisting of hydrogen, halogen, C₁-C₃ alkyl and C₁-C₃ haloalkyl;
R² is selected from the group consisting of hydrogen, halogen, C₁-C₃ alkyl, C₁-C₃ alkoxy-, C₁-C₃haloalkoxy- and C₁-C₃ haloalkyl;
R³ is selected from the group consisting of hydrogen, halogen, —CN, nitro, C₁-C₄ alkyl, C₂-C₄ alkenyl-, C₂-C₄ alkynyl-, C₁-C₄ haloalkyl-, C₁-C₄ alkoxy-, C₁-C₄ haloalkoxy- and —S(O)ₙC₁-C₄ alkyl;
R⁴ is selected from the group consisting of hydrogen, halogen, —CN, nitro, C₁-C₄ alkyl, C₂-C₄ alkenyl-, C₂-C₄ alkynyl-, C₁-C₄ haloalkyl-, C₁-C₄ alkoxy-, C₁-C₄ haloalkoxy- and —S(O)ₙC₁-C₄ alkyl;
each R⁵ is independently selected from the group consisting of halogen, —CN, nitro, C₁-C₄ alkyl, C₂-C₄ alkenyl, C₂-C₄ alkynyl, C₁-C₄ haloalkyl, C₁-C₄ alkoxy-, C₁-C₄ haloalkoxy-, —S(O)ₚC₁-C₄ alkyl and —S(O)ₚC₁-C₄ haloalkyl;
X¹ is —(CR¹⁰R¹¹)_{q}—
Z¹ is N or CR⁷;
Z² is N or CR⁸;
R⁷ is selected from the group consisting of hydrogen, C₁-C₄ alkyl, halogen, —CN, nitro, C₂-C₄ alkenyl, C₂-C₄ alkynyl, C₁-C₄ haloalkyl, C₁-C₄ alkoxy-, C₁-C₄ haloalkoxy-, —S(O)ₚC₁-C₄ alkyl and —S(O)ₚC₁-C₄ haloalkyl;
R⁸ is selected from the group consisting of hydrogen, C₁-C₄ alkyl, halogen, —CN, nitro, C₂-C₄ alkenyl, C₂-C₄ alkynyl, C₁-C₄ haloalkyl, C₁-C₄ alkoxy-, C₁-C₄ haloalkoxy-, —S(O)ₚC₁-C₄ alkyl and —S(O)ₚC₁-C₄ haloalkyl;
R⁹ is selected from the group consisting of C₁ haloalkyl-, C₁-C₆ alkyl-C₁ haloalkyl-, hydroxy, C₁-C₃ alkoxy-, —C(O)—R¹⁴, —CN, —C₃-C₆ cycloalkyl, —C(R¹²)═N—O—R¹³, —C(R¹²)═C—N(R¹²)(R¹²), —CR¹⁷ (—CH₂CH₂—), —CR¹⁶ (—CH₂OCH₂—), —CR¹⁶ (—CH₂CH₂OCH₂—), —CR¹⁶ (—CH₂CH₂OCH₂CH₂—), —CR¹⁶ (—CH₂CH₂N(R¹⁵)CH₂CH₂—), phenyl and 5- or 6-membered heteroaryl wherein the phenyl and heteroaryl are optionally substituted by 1 or 2 substituents independently selected from the group consisting of halogen, CN, C₁-C₂ alkyl, C₁-C₂ haloalkyl, C₁-C₂ alkoxy- and C₁-C₂ haloalkoxy-,
with the proviso that if R¹⁰ and R¹¹ are hydrogen and q=1 then R⁹ is not pyrimidin-2-yl and if R₉ is C₁ haloalkyl- or C₁-C₆ alkyl-C₁ haloalkyl-then q=0;
R¹⁰ and R¹¹ are independently selected from the group consisting of hydrogen, hydroxy, halogen and C₁-C₃ alkyl;
R¹² is hydrogen or C₁-C₃ alkyl;
R¹³ is selected from the group consisting of hydrogen, C₁-C₆ alkyl, C₁-C₆ haloalkyl, C₂-C₆ alkenyl and —(CH₂) C₃-C₆ cycloalkyl;
R¹⁴ is selected from the group consisting of hydrogen, C₁-C₆ alkyl, —N(R¹²) C₁-C₆ alkyl, phenyl and a 5- or 6-membered heteroaryl wherein the phenyl and heteroaryl are optionally substituted by 1 or 2 substituents independently selected from the group consisting of halogen, CN, C₁-C₂ alkyl, C₁-C₂ haloalkyl, C₁-C₂ alkoxy- and C₁-C₂ haloalkoxy-;
R¹⁵ is C₁C₆ alkoxy- or —C(O) C₁-C₆ alkyl;
R¹⁶ is selected from the group consisting of hydrogen, halogen, CN and C₁-C₂ alkoxy-;
R¹⁷ is selected from the group consisting of halogen, CN and C₁-C₂ alkoxy-;
n=0, 1 or 2;
p=0, 1 or 2 and
q=0, 1, 2, 3 or 4.

2. A compound according to claim 1, wherein R¹ and R² are both hydrogen.

3. A compound according to claim 1, wherein Y1 is CR³ and Y² is N.

4. A compound according to claim 3, wherein R³ is chloro.

5. A compound according to claim 1, wherein n=0.

6. A compound according to claim 1, wherein Z¹ is CH and Z² is CH.

7. A compound according to claim 1, wherein Z¹ is N and Z² is N.

8. A compound according to claim 1, wherein R⁹ is —C(O)—R¹⁴.

9. A compound according to claim 1, wherein R⁹ is selected from the group consisting of —C(R12)═N—O—R¹³, —CR¹⁶(—CH₂OCH₂—), CR¹⁶(—CH₂CH₂OCH₂—), —CR¹⁶(—CH₂CH₂OCH₂CH₂—), phenyl and a 5- or 6-membered heteroaryl wherein the phenyl and heteroaryl are optionally substituted by 1 or 2 substituents independently selected from the group consisting of halogen, CN, C₁-C₂ alkyl, C₁-C₂ haloalkyl, C₁-C₂ alkoxy- and C₁-C₂ haloalkoxy-, with the proviso that if R¹⁰ and R¹¹ are hydrogen and q=1 then R⁹ is not pyrimidin-2-yl.

10. A compound according to claim 1, wherein R⁹ is —C₃-C₆ cycloalkyl.

11. A herbicidal composition comprising a compound according to claim 1 and an agriculturally acceptable formulation adjuvant.

12. A herbicidal composition according to claim 11, further comprising at least one additional pesticide.

13. A herbicidal composition according to claim 12, wherein the additional pesticide is a herbicide or herbicide safener.

14. A method of controlling weeds at a locus comprising application to the locus of a weed controlling amount of a composition according to claim 11.

15. Use of a compound of Formula (I) as defined in claim 1 as a herbicide.
